(19) United States
(12) Patent Application Publication
(10) Pub. No.: US 2026/0274825 A1
(43) Pub. Date: Sep. 17, 2026

(54) PIKfyve Kinase Inhibitors

(71) Applicant: Dunad Therapeutics Ltd., Duxford (GB)

(72) Inventors: Barry John TEOBALD, Duxford (GB); Lou CHUPAK, Duxford (GB); Mirco MENICONI, Duxford (GB); Graham L. SIMPSON, Duxford (GB); Laura D'AGOSTINO, Duxford (GB)

(21) Appl. No.: 19/678,660

(22) Filed: May 15, 2026

Related U.S. Application Data
(63) Continuation of application No. PCT/US25/59639, filed on Dec. 15, 2025.
(60) Provisional application No. 63/734,432, filed on Dec. 16, 2024.
(60) Provisional application No. 63/832,635, filed on Jun. 30, 2025.

(51) Int. Cl. C07D 403/14; C07D 401/14

(52) U.S. Cl. CPC C07D 403/14; C07D 401/14

(57) ABSTRACT

The present invention relates to compounds of Formula I useful as inhibitors of phosphatidylinositol-3-phosphate 5-kinase (PIKfyve) as well as their use for treating diseases and disorders associated with PIKfyve. In particular, the PIKfyve inhibitors of the invention comprise a functional group modification that allows for a covalent interaction with PIKfyve. In some embodiments, a compound of the invention forms a covalent bond with an amino acid residue of the PIKfyve protein.

### RELATED APPLICATIONS

This application is a continuation of International Application No. PCT/US2025/059639, which designated the United States and was filed on Dec. 15, 2025, published in English, which claims the benefit of U.S. Provisional Application No. 63/734,432 filed Dec. 16, 2024, and U.S. Provisional Application No. 63/832,635 filed Jun. 30, 2025. The entire contents of the above-referenced applications are incorporated by reference herein.

### BACKGROUND OF THE INVENTION

Phosphatidylinositol-3-phosphate 5-kinase (PIKfyve) is named after its function and domain structure (phosphoinositide kinase for five position containing a FYVE finger) is a lipid kinase that phosphorylates phosphatidylinositol-3-phosphate (PI(3)P), producing PI(3,5)P₂, which is involved in cellular processes including membrane trafficking and cytoskeletal reorganization. Inhibiting PIKfyve can alter both lipid levels and TFEB-driven transcription leading to changes in autophagy and resultant increases in autophagic flux and exocytosis and downstream degradation/clearance of protein aggregates (Hung et al., Staats et al., Shi et al., Soares et al., Lucus-Del-Pozo et al.). Inhibiting PIKfyve may be useful in the treatment of various diseases or conditions such as viral infections, autoimmune diseases, inflammatory diseases, cancer, pain, or many neurological diseases. Thus, there is a need for improved inhibitors of PIKfyve.

### SUMMARY OF THE INVENTION

The present invention relates to compounds useful as inhibitors of phosphatidylinositol-3-phosphate 5-kinase (PIKfyve) as well as their use for treating diseases and disorders associated with PIKfyve. In particular, the PIKfyve inhibitors of the invention comprise a functional group modification that allows for a covalent interaction with PIKfyve. In some embodiments, a compound of the invention forms a covalent bond with an amino acid residue of the PIKfyve protein.

One of ordinary skill in the art will recognize that certain reactive functional groups can act as “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified), present in or near the binding pocket of the PIKfyve target protein, thereby inhibiting the PIKfyve protein. In some embodiments, the covalent binding of the compound of the invention to the amino acid of the PIKfyve protein is reversible. In some embodiments, the covalent binding of the compound of the invention to the amino acid of the PIKfyve protein is irreversible.

It will be appreciated that in some embodiments the Linker-Warhead group ((Q)ₚ-W), as defined and described herein, provides such warhead groups for covalently, and reversibly, inhibiting the PIKfyve protein.

It will be appreciated that in some embodiments the Linker-Warhead group ((Q)ₚ-W), as defined and described herein, provides such warhead groups for covalently, and irreversibly, inhibiting the PIKfyve protein.

In some embodiments, a compound of the invention forms a covalent bond with a sulfur atom of a cysteine residue of the PIKfyve protein. In embodiments, the covalent bonding between the compound of the invention and the cysteine residue of PIKfyve is irreversible.

Any suitable method for determining a reversible or an irreversible (e.g., covalently reversible or irreversible) interaction between a compound of the invention and the PIKfyve protein can be used. Methods for identifying if a compound is acting as a reversible or an irreversible inhibitor are known to one of ordinary skill in the art.

In one aspect, the present invention provides a compound of formula (I):

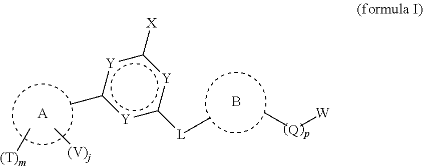

or a pharmaceutically acceptable salt thereof, wherein Ring A is a 5- or 6-membered aromatic ring having 0-3 heteroatoms selected from N, O, or S, preferably 1-3 heteroatoms, preferably 2 heteroatoms, preferably 3 heteroatoms, preferably wherein the heteroatom is N;
T is selected from halogen, C₁-C₆ alkyl, alkoxy or an aryl or heteroaryl having 0-3 heteroatoms selected from N, O, or S, preferably 0, wherein the C₁-C₆ alkyl, C₁-C₆ alkoxy, or the 6-membered aryl or heteroaryl is optionally substituted; preferably T is a 6 membered aryl or heteroaryl ring;
V is a C₁-C₃ alkyl, wherein the C₁-C₃ alkyl is optionally substituted; each Y is independently CR₁, N, O, or S, provided at least one Y is N, O, or S(preferably, N), wherein R₁ is H, D, halogen (preferably Cl, F), or substituted or unsubstituted C₁-C₆ alkyl;
X is a 5- to 8-membered heterocyclic ring comprising at least one heteroatom selected from N or O, wherein the ring is optionally substituted, wherein the ring is optionally fused or bridged (e.g., forming a bicyclic ring);
L is —X₁—(CR₃R₄)ₖ—, —(CR₃R₄)ₖ—, —X₁—(CR₃R₄) K—X₁—, —(CR₃R₄)ₖ—X₁—, —(CR₃R₄) k-X₁—(CR₃R₄) k-; each X₁ is independently selected from —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂—, and —NR₂C(Z₁) NR₂—; wherein each occurrence of Z₁ is independently O or NR₆, preferably Z₁ is O; preferably —O—, —C(O)—, —C(O)O—, —OCO—, —S—, —S(O)₂—, —S(O)—, —S(O)(NR₂)—, —S(O)₂ (NR₂)—, —NR₂—, —NR₂C(O)—, and —C(O)NR₂—;
R₂ is hydrogen, substituted or unsubstituted C₁₋₆ alkyl, substituted or unsubstituted C₃₋₇ cycloalkyl, or substituted or unsubstituted C₃₋₇ heterocycloalkyl;
R₆ is hydrogen, substituted or unsubstituted C₁₋₆ alkyl, substituted or unsubstituted C₃₋₇ cycloalkyl, substituted or unsubstituted C₃₋₇ heterocycloalkyl or acyl; or each occurrence of R₃ and R₄ are independently H, halogen, hydroxy, substituted or unsubstituted alkyl, substituted or unsubstituted alkenyl, substituted or unsubstituted alkynyl, or substituted or unsubstituted alkoxy; alternatively, at least one occurrence of R₃ and R₄ forms an optionally substituted cycloalkyl or heterocycloalkyl (preferably, oxetane, azetidine, pyrrolidine, or piperidine); alternatively, when X₁ comprises-NR₂—, any two of one R₃, one R₄, and R₂ forms an optionally substituted heterocycloalkyl;
Ring B is a substituted or unsubstituted 5- or 6-membered aromatic ring having 0-3 heteroatoms selected from N, O, or S, preferably 1-3 heteroatoms, preferably 2 heteroatoms, preferably 3 heteroatoms, preferably wherein the heteroatom is N; alternatively, when L comprises —(CR₃R₄)ₖ—, B and one adjacent occurrence of R₃ forms a bicyclic ring;
Q is selected from —CR₃R₄—, —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂—, and —NR₂C(Z₁) NR₂—; wherein each occurrence of Z₁ is independently O or NR₆, preferably Z₁ is O; Q is preferably —C(O)—, —CR₃R₄—, —O—, —S(O)₂—, —NR₂—, —C(O)O—, —OC(O)—, —C(O)NR₂—, —NR₂C(O)—, —S(O)—, —S(O)(NR₂)—, —S(O)₂ (NR₂)—, —OC(O)NR₂—, —NR₂C(O)O—;
W is substituted or unsubstituted aryl or substituted or unsubstituted heteroaryl, wherein the heteroaryl comprises one or more heteroatoms selected from N, O, or S, wherein at least one substituent is a halogen (e.g., F or Cl), —SR₂, —S(Z₁)ₓR₂—S(Z₁)ₓNR₂R₆, —NR₂S(Z₁)ₓR₆, —OS(Z₁)ₓR₆, substituted or unsubstituted alkene or substituted or unsubstituted alkyne (preferably alkyl- or fluoro-substituted alkene or alkyne); or when W is attached to N, W is substituted or unsubstituted alkenoyl or substituted or unsubstituted alkynoyl, substituted or unsubstituted alkenesulfonyl; substituted or unsubstituted alkenesulfinyl; substituted or unsubstituted alkynesulfonyl, substituted or unsubstituted alkynesulfinyl, W is preferably a substituted or unsubstituted aryl, a substituted or unsubstituted heteroaryl, wherein the heteroaryl comprises one or more heteroatoms selected from N, O, or S;
m is 0 or 1;
j is 0 or 1;
p is 0 or 1;
k is 0, 1, 2, 3, 4, 5, or 6 and
x is 1 or 2.

In another aspect, described herein is a compound wherein the compound is a compound from Table 1.

In another aspect, described herein is a pharmaceutical composition comprising a compound as described herein, or a salt thereof and one or more of pharmaceutically acceptable carriers.

In another aspect, described herein is a method of inhibiting PIKfyve in a subject (such as a human subject) in need thereof comprising administering an effective amount of a compound of the present invention to the subject.

In yet another aspect, described herein is a method for treating a disease or disorder associated with PIKfyve in a human subject in need thereof comprising administering an effective amount of a compound of the present invention to the subject.

### DETAILED DESCRIPTION OF THE INVENTION

Provided herein are compounds or pharmaceutically acceptable salts thereof, and compositions that inhibit phosphatidylinositol-3-phosphate 5-kinase (PIKfyve). As such, in one aspect, the compounds and compositions provided herein are useful in inhibiting PIKfyve and/or for treating a disease or disorder associated with PIKfyve in a subject (such as a human subject) in need thereof.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting. The terminology used herein is for the purpose of describing particular embodiments only and is not intended to be limiting.

The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The term “treat,” “treated,” “treating,” or “treatment” refers to an approach for obtaining beneficial or desired results including but not limited to therapeutic benefit. By therapeutic benefit is meant eradication or amelioration of the underlying state, disease, or disorder being treated. Also, a therapeutic benefit includes the diminishment or alleviation of at least one symptom associated or caused by the state, disorder or disease being treated.

As used herein, the term “prevent” or “prevention” means no disorder or disease development if none had occurred, or no further disorder or disease development if there had already been development of the disorder or disease. Also considered is the ability of one to prevent one, some, or all of the symptoms associated with the disorder or disease.

As used herein, the term “patient,” “individual” or “subject” includes a mammal. The mammal can be e.g., any mammal, e.g., a human, a primate, a mouse, a rat, a dog, a cat, a cow, a horse, a goat, a camel, a sheep, or a pig. Preferably, the mammal is a human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. L ists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, sublingual, buccal, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “administration” of a compound or a composition to a subject includes any route of introducing or delivering the agent to a subject to perform its intended function. Administration can be carried out by any suitable oral or non-oral route as described herein. Administration includes self-administration and the administration by another.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

The term “alkyl”, unless otherwise specified, refers to a straight or branched hydrocarbon chain radical consisting solely of carbon and hydrogen atoms, containing no unsaturation, having from one to eight carbon atoms, and which is attached to the rest of the molecule by a single bond, e.g. methyl, ethyl, n-propyl, 1-methylethyl (isopropyl), n-butyl, n-pentyl, and 1,1-dimethylethyl (t-butyl). The term “C₁₋₆ alkyl” refers to an alkyl group as defined above having 1 to 6 carbon atoms. The term “C₁₋₃ alkyl” refers to an alkyl group as defined above having 1 to 3 carbon atoms. In appropriate circumstances, the term “alkyl” refers to a hydrocarbon chain radical as mentioned above which is bivalent.

The term “alkenyl”, unless otherwise specified, refers to an aliphatic hydrocarbon group containing one or more carbon-carbon double bonds and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “C₂₋₆ alkenyl” refers to an alkenyl group as defined above having 2 to 6 carbon atoms. In appropriate circumstances, the term “alkenyl” refers to a hydrocarbon group as mentioned above which is bivalent.

The term “alkynyl”, unless otherwise specified, refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butynyl. The term “C₂₋₆ alkynyl” refers to an alkynyl group as defined above having 2 to 6 carbon atoms. In appropriate circumstances, the term “alkynyl” refers to a hydrocarbyl radical as mentioned above which is bivalent.

The term “alkoxy” unless otherwise specified, denotes an alkyl, cycloalkyl, or cycloalkylalkyl group as defined herein attached via an oxygen linkage to the rest of the molecule. The term “substituted alkoxy” refers to an alkoxy group where the alkyl constituent is substituted (i.e., —O-(substituted alkyl)). For example, “alkoxy” refers to the group-O-alkyl, including from 1 to 8 carbon atoms of a straight, branched, cyclic configuration and combinations thereof attached to the parent structure through an oxygen atom. Examples include methoxy, ethoxy, propoxy, isopropoxy, cyclopropyloxy, and cyclohexyloxy. In appropriate circumstances, the term “alkoxy” refers to a group as mentioned above which is bivalent.

The term “cycloalkyl”, unless otherwise specified, denotes a non-aromatic mono or multicyclic ring system of about 3 to 12 carbon atoms (unless otherwise specified) such as cyclopropyl, cyclobutyl, cyclopentyl, and cyclohexyl. Examples of multicyclic cycloalkyl groups include perhydronaphthyl, adamantyl and norbornyl groups, bridged cyclic groups, and spirobicyclic groups, e.g., spiro[4.4]non-2-yl. The term “C₃₋₆ cycloalkyl” refers to a cycloalkyl group as defined above having 3 to 6 carbon atoms.

The term “cycloalkylalkyl”, unless otherwise specified, refers to a cyclic ring-containing radical containing in the range of about 3 up to 8 carbon atoms (unless otherwise specified) directly attached to an alkyl group which is then attached to the main structure at any carbon from the alkyl group, such as cyclopropylmethyl, cyclobutylethyl, and cyclopentylethyl.

The term “cycloalkenyl”, unless otherwise specified, refers to cyclic ring-containing radicals containing in the range of about 3 to 8 carbon atoms with at least one carbon-carbon double bond such as cyclopropenyl, cyclobutenyl, and cyclopentenyl.

The term “cycloalkenylalkyl” refers to a cycloalkenyl group directly attached to an alkyl group which is then attached to the main structure at any carbon from the alkyl group.

The term “aryl”, unless otherwise specified, refers to aromatic radicals having in the range of 6 to 20 carbon atoms such as phenyl, naphthyl, tetrahydronaphthyl, indanyl, and biphenyl. Aryl groups are intended herein to include fused or bicyclic rings and heteroaryl groups, unless otherwise stated.

The term “arylalkyl”, unless otherwise specified, refers to an aryl group as defined above directly bonded to an alkyl group as defined above, e.g., —CH₂C₆H₅ and —C₂H₅C₆H₅.

The term “heterocyclic ring”, unless otherwise specified, refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized. The heterocyclic ring radical may be attached to the main structure at any heteroatom or carbon atom. Examples of such heterocyclic radicals include, but are not limited to, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, oxazolinyl, morpholinyl, quinuclidinyl, octahydroindolyl, octahydroisoindolyl, decahydroisoquinolyl, tetrahydrofuryl, tetrahydropyranyl, thiamopholinyl, thiamorpholinyl sulfoxide, thiamopholinyl sulfone, and dioxaphospholanyl.

The term “heterocyclyl”, unless otherwise specified, refers to a heterocyclic ring radical as defined above. The heterocylcyl ring radical may be attached to the main structure at any heteroatom or carbon ring atom. In appropriate circumstances, the term “heterocyclyl” refers to a hydrocarbon chain radical as mentioned above which is bivalent. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heterocyclylalkyl”, unless otherwise specified, refers to a radical of the formula-R^{w}-heterocyclyl where R^{w} is an alkylene chain as defined herein. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined herein for an alkylene chain. The heterocyclyl part of the heterocyclylalkyl radical is optionally substituted as defined herein for a heterocyclyl group.

The term “heteroaryl”, unless otherwise specified, refers to an optionally substituted 5 to 14 member aromatic ring having one or more heteroatoms selected from N, O, and S as ring atoms. The heteroaryl may be a mono-, bi- or tricyclic ring system. Examples of such “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl, isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, pyridazinyl, oxazolidinyl, triazolyl, indanyl, isoxazolyl, isoxazolidinyl, thiazolinyl, thiazolidinyl, isothiazolyl, isothiazolidinyl, isoindolyl, indolinyl, isoindolinyl, benzimidazolyl, thiadiazolyl, benzopyranyl, thienyl, benzothienyl, oxadiazolyl, chromanyl, and isochromanyl. The term “5 or 6-membered heteroaryl” refers to a heteroaryl having 5- or 6-ring atoms. The term “5-6 or 6-5 membered bicyclic heteroaryl” refers to a bicyclic heteroaryl with a five-membered ring fused to a six-membered ring, where the 5-membered ring is bound to the rest of the molecule (referred to as a “5-6 membered bicyclic heteroaryl”) or the 6-membered ring is bound to the rest of the molecule (referred to as a “6-5 membered bicyclic heteroaryl”). The term “6-6 membered bicyclic heteroaryl” refers to a bicyclic heteroaryl with a six membered ring fused to a another six-membered ring, where one of the 6-membered rings is bound to the rest of the molecule. The heteroaryl ring radical may be attached to the main structure at any heteroatom or carbon atom. The term “substituted heteroaryl” also includes ring systems substituted with one or more oxide (—O—) substituents, such as pyridinyl N-oxides.

The term “heteroarylalkyl”, unless otherwise specified, refers to a radical of the formula-R^{w}-heteroaryl, where R^{w} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined herein for an alkylene chain. The heteroaryl part of the heteroarylalkyl radical is optionally substituted as defined herein for a heteroaryl group.

The term “cyclic ring” refers to a cyclic ring containing 3 to 10 carbon atoms, unless otherwise specified.

The term “substituted” unless otherwise specified, refers to substitution with any one or any combination of the following substituents which may be the same or different and are independently selected from hydrogen, hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted cycloalkenylalkyl, substituted or unsubstituted heteroaryl, substituted or unsubstituted heteroarylalkyl, substituted or unsubstituted heterocyclic ring, substituted heterocyclylalkyl ring, substituted or unsubstituted guanidine, —COOR^{a}, —C(O)R^{a}, —C(S)R^{a}, —C(O)NR^{a}R^{b}, —C(O)NR^{a}(OR^{b}), —NR^{b}R^{c}, —NR^{a}CONR^{b}R^{c}, —N(R^{a})SOR^{b}, —N(R^{a})SO₂R^{b}, ═N—NR^{a}R^{b}, —NR^{a}C(O)OR^{b}, —NR^{a}R^{b}, —NR^{a}C(O)R^{b}, —NR^{a}C(S)R^{b}—NR^{a}C(S)R^{b}R^{c}, —SONR^{a}R^{b}, —SO₂NR^{a}R^{b}, —OR^{a}, —OR^{a}C(O)R^{b}R^{c}, —OR^{a}C(O)OR^{b}, —OC(O)R^{a}, —OC(OR^{a}R^{b}, —R^{a}NR^{b}C(O)R^{c}, —ROR^{b}, —R^{a}C(O)OR^{b}, —R^{a}C(O)R^{b}R^{c}, —R^{a}C(O)R^{a}, —ROC(O)R^{b}, —SR^{a}, —SOR^{a}, —SO₂R^{a}, and —ONO₂, wherein R^{a}, R^{b} and R^{c} in each of the above groups can be hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkylalkyl, substituted or unsubstituted cycloalkenyl, substituted or unsubstituted amino, substituted or unsubstituted heteroaryl, substituted or unsubstituted heteroarylalkyl, substituted or unsubstituted heterocyclic ring, or substituted heterocyclylalkyl ring, or any two of R^{a}, R^{b} and R^{c} may be joined to form a substituted or unsubstituted saturated or unsaturated 3-10 membered ring, which may optionally include heteroatoms which may be the same or different and are selected from O, NR^{a}(e.g., R^{a} can be hydrogen or C₁₋₆ alkyl) or S. Particularly useful substituents include detectable labels to assist in identifying the compound in a sample or tissue. Examples of labels include ligands (e.g., biotin), fluorescence labels (e.g., fluorescein), radiolabels (C14), enzyme labels (alkaline phosphatase), chemiluminescence labels (e.g., luminol), and the like. The labels can be conjugated to the compound through a linker or spacer (such as an alkyl, amide or PEG linker). Substitution or the combinations of substituents envisioned by this invention are preferably those that result in the formation of a stable or chemically feasible compound. The term stable as used herein refers to the compounds or the structure that are not substantially altered when subjected to conditions to allow for their production, detection and preferably their recovery, purification and incorporation into a pharmaceutical composition. The substituents in the aforementioned “substituted” groups cannot be further substituted, Aryl groups are intended herein to include fused or bicyclic rings and heteroaryl groups, unless otherwise stated. For example, when the substituent on “substituted alkyl” is “substituted aryl”, the substituent on “substituted aryl” cannot be “substituted alkenyl”.

The term “halo”, “halide”, or, alternatively, “halogen” means fluoro, chloro, bromo or iodo.

The terms “haloalkyl”, “haloalkenyl”, “haloalkynyl”, and “haloalkoxy” include alkyl, alkenyl, alkynyl and alkoxy structures that are substituted with one or more halogen groups or with combinations thereof. For example, the terms “fluoroalkyl” and “fluoroalkoxy” include haloalkyl and haloalkoxy groups, respectively, in which the halogen is fluorine.

Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures.

Additionally, the instant invention also includes the compounds which differ only in the presence of one or more isotopically enriched atoms for example replacement of hydrogen with deuterium or tritium, the replacement of a carbon by ¹³C- or ¹⁴C-enriched carbon, or the replacement of a nitrogen by ¹⁵N. The compounds of the present invention may also contain unnatural proportions of atomic isotopes at one or more of atoms that constitute such compounds. For example, the compounds may be radiolabeled with radioactive isotopes, such as for example tritium, iodine-125 (¹²⁵I) or carbon-14 (¹⁴C). All isotopic variations of the compounds of the present invention, whether radioactive or not, are encompassed within the scope of the present invention. It is appreciated that, in some instances, deuterium is specifically identified and is a preferred point of deuteration at this position. This is not intended to infer that other deuterated compounds are not envisioned. In fact, deuterated compounds are included.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Compounds

In one aspect, the invention provides a compound of formula (I):

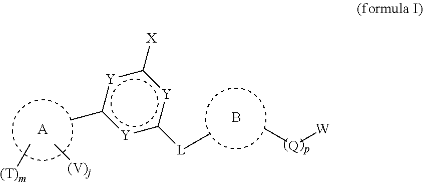

or a pharmaceutically acceptable salt thereof, wherein Ring A is a 5- or 6-membered aromatic ring having 0-3 heteroatoms selected from N, O, or S, preferably 1-3 heteroatoms, preferably 2 heteroatoms, preferably 3 heteroatoms, preferably wherein the heteroatom is N;
T is selected from halogen, C₁-C₆ alkyl, alkoxy or an aryl or heteroaryl having 0-3 heteroatoms selected from N, O, or S, preferably 0, wherein the C₁-C₆ alkyl, C₁-C₆ alkoxy, or the 6-membered aryl or heteroaryl is optionally substituted; preferably T is a 6 membered aryl or heteroaryl ring;
V is a C₁-C₃ alkyl, wherein the C₁-C₃ alkyl is optionally substituted; each Y is independently CR₁, N, O, or S, provided at least one Y is N, O, or S(preferably, N), wherein R₁ is H, D, halogen (preferably Cl, F), or substituted or unsubstituted C₁-C₆ alkyl;
X is a 5- to 8-membered heterocyclic ring comprising at least one heteroatom selected from N or O, wherein the ring is optionally substituted, wherein the ring is optionally fused or bridged (e.g., forming a bicyclic ring);
L is —X₁—(CR₃R₄)ₖ—, —(CR₃R₄)ₖ—, —X₁—(CR₃R₄)ₖ—X₁—, —(CR₃R₄)ₖ—X₁—, —(CR₃R₄)—X₁—(CR₃R₄) k-; each X₁ is independently selected from —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂—, and —NR₂C(Z₁) NR₂—; wherein each occurrence of Z₁ is independently O or NR₆, preferably Z₁ is O; preferably-O—, —C(O)—, —C(O)O—, —OCO—, —S—, —S(O)₂—, —S(O)—, —S(O)(NR₂)—, —S(O)₂ (NR₂)—, —NR₂—, —NR₂C(O)—, and —C(O)NR₂—;
R₂ is hydrogen, substituted or unsubstituted C₁₋₆ alkyl, substituted or unsubstituted C₃₋₇ cycloalkyl, or substituted or unsubstituted C₃₋₇ heterocycloalkyl;
each occurrence of R₃ and R₄ are independently H, halogen, hydroxy, substituted or unsubstituted alkyl, substituted or unsubstituted alkenyl, substituted or unsubstituted alkynyl, or substituted or unsubstituted alkoxy; alternatively, at least one occurrence of R₃ and R₄ forms an optionally substituted cycloalkyl or heterocycloalkyl (preferably, oxetane, azetidine, pyrrolidine, or piperidine); alternatively, when X₁ comprises —NR₂—, any two of one R₃, one R₄, and R₂ forms an optionally substituted heterocycloalkyl;
Ring B is a substituted or unsubstituted 5- or 6-membered aromatic ring having 0-3 heteroatoms selected from N, O, or S, preferably 1-3 heteroatoms, preferably 2 heteroatoms, preferably 3 heteroatoms, preferably wherein the heteroatom is N; alternatively, when L comprises —(CR₃R₄)ₖ—, B and one adjacent occurrence of R₃ forms a bicyclic ring;
Q is selected from —CR₃R₄—, —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂— and —NR₂C(Z₁) NR₂; wherein each occurrence of Z₁ is independently O or NR₆, preferably Z₁ is O; Q is preferably-C(O)—, —CR₃R₄—, —O—, —S(O)₂—, —NR₂—, —C(O)O—, —OC(O)—, —C(O)NR₂—, —NR₂C(O)—, —S(O)—, —S(O)(NR₂)—, —S(O)₂ (NR₂)—, —OC(O)NR₂—, —NR₂C(O)O—;
W is substituted or unsubstituted aryl or substituted or unsubstituted heteroaryl, wherein the heteroaryl comprises one or more heteroatoms selected from N, O, or S, wherein at least one substituent is a halogen (e.g., F or Cl), —SR₂, —S(Z₁); R₂—, —S(Z₁)ₓNR₂R₆, —NR₂S(Z₁)ₓR₆, —OS(Z₁)ₓR₆, substituted or unsubstituted alkene or substituted or unsubstituted alkyne (preferably alkyl- or fluoro-substituted alkene or alkyne); or when W is attached to N, W is substituted or unsubstituted alkenoyl or substituted or unsubstituted alkynoyl, substituted or unsubstituted alkenesulfonyl; substituted or unsubstituted alkenesulfinyl; substituted or unsubstituted alkynesulfonyl, substituted or unsubstituted alkynesulfinyl, preferably W is a substituted or unsubstituted aryl, a substituted or unsubstituted heteroaryl, wherein the heteroaryl comprises one or more heteroatoms selected from N, O, or S;
m is 0 or 1;
j is 0 or 1;
p is 0 or 1;
k is 0, 1, 2, 3, 4, 5, or 6 and
x is 1 or 2.

In one embodiment, ring A is a 5-membered aromatic ring having 1-3 heteroatoms selected from N, O, or S. Preferably, ring A comprises 2-3 heteroatoms. Preferably, ring A comprises 2 heteroatoms. Preferably, ring A comprises 3 heteroatoms. Preferably, the heteroatom is N.

In embodiments, ring A is selected from:

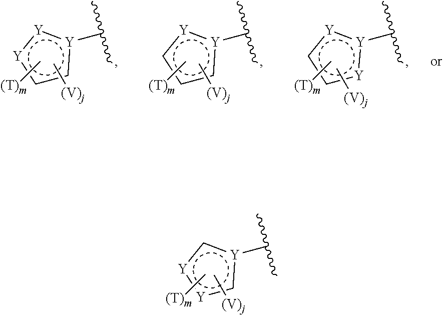

(the squiggly lines indicate the point of attachment to the rest of the molecule).

In embodiments, ring A is selected from:

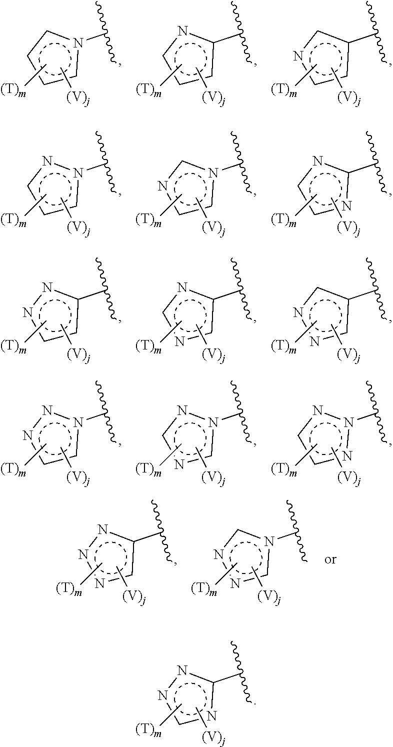

In embodiments, Ring A is selected from:

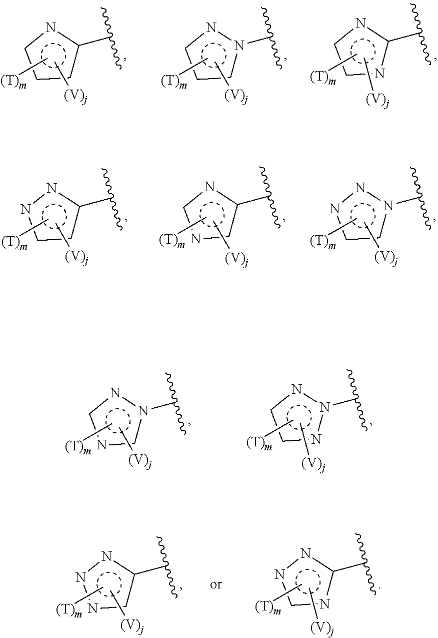

In embodiments, Ring A is selected from:

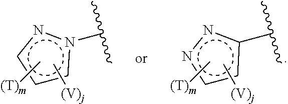

In embodiments, Ring A is:

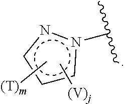

In some of any of the embodiments described herein, m is 1 and T is substituted or unsubstituted phenyl or substituted or unsubstituted pyridine, substituted or unsubstituted pyrazole or substituted or unsubstituted imidazole.

In some of any of the embodiments described herein, T is phenyl, a halogen-substituted phenyl, an alkyl-substituted phenyl, a halogenated alkyl-substituted phenyl, or an alkoxy-substituted phenyl.

In some of any of the embodiments described herein, T is selected from phenyl, 2-methylphenyl, 3-methylphenyl, 4-methylphenyl, 3-fluorophenyl, and 3-chlorophenyl.

In some of any of the embodiments described herein, T is pyridine, a halogen-substituted pyridine, an alkyl-substituted pyridine, a halogenated alkyl-substituted pyridine, or an alkoxy-substituted pyridine.

In some of any of the embodiments described herein, T is selected from pyridine, 3-methylpyridine, 3-fluoro pyridine, and 3-chloro pyridine.

In some of any of the embodiments described herein, the ring A moiety, e.g.,

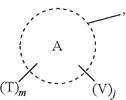

is selected from:

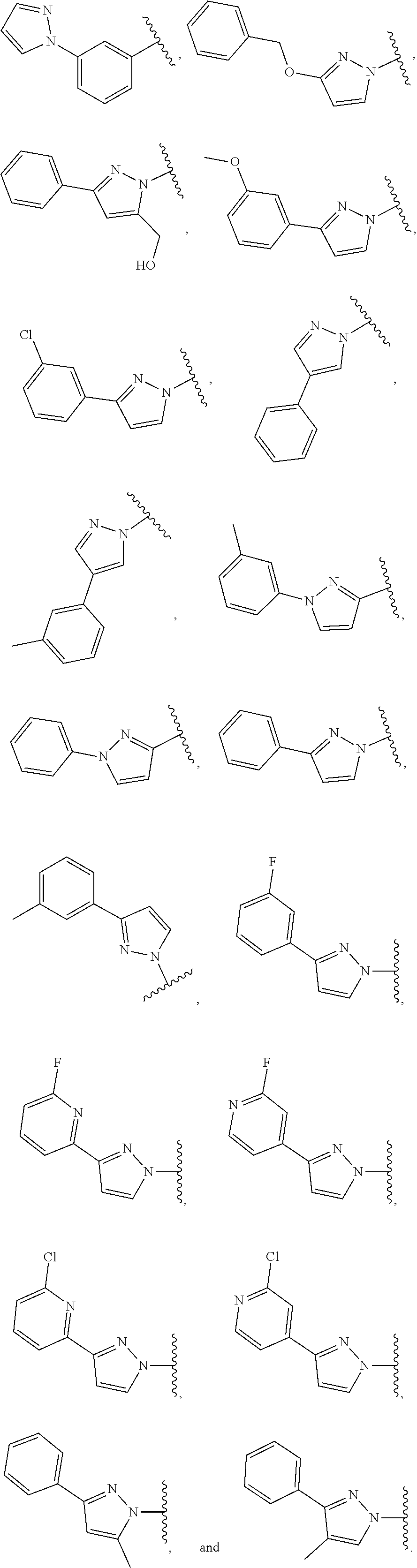

In some of any of the embodiments described herein, the

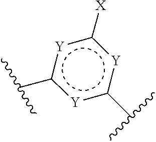

moiety is selected from:

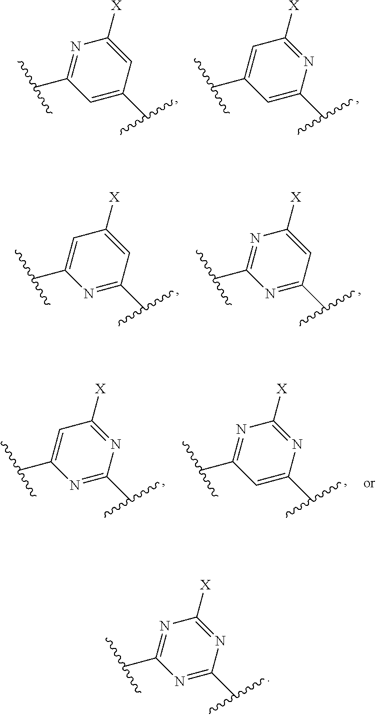

In some of any of the embodiments described herein, the

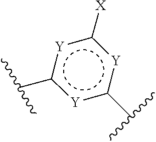

moiety is selected from:

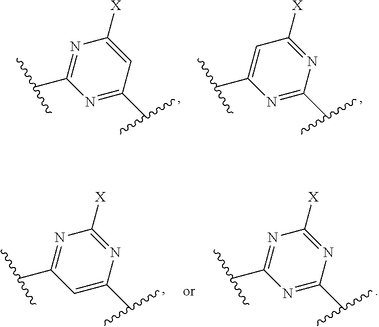

In some of any of the embodiments described herein, X is selected from:

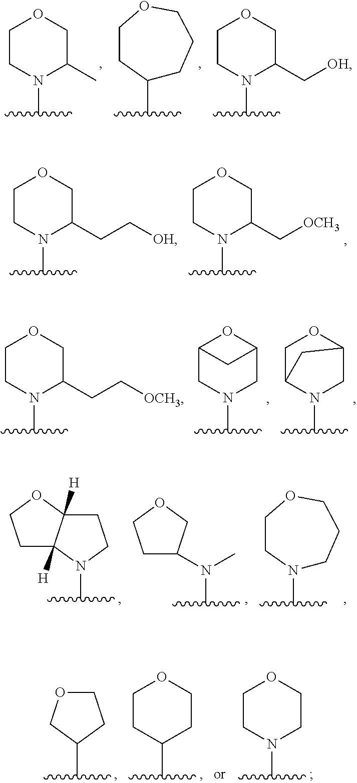

wherein each of the rings above is optionally substituted, and the substituents are independently selected from halogen, —OH, a C₁₋₄ alkyl, and a C₁₋₄ alkoxy.

In some of any of the embodiments described herein, X is selected from:

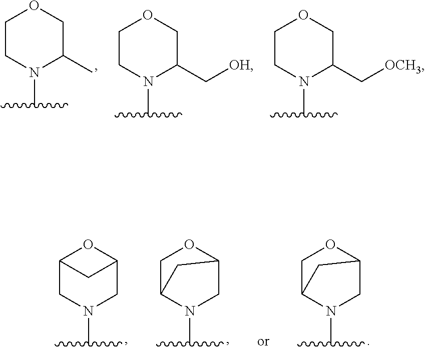

In some of any of the embodiments described herein, X is:

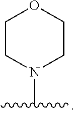 [SMILES: *N1CCOCC1]

In some of any of the embodiments described herein, L is —X₁—(CR₃R₄)ₖ—.

In embodiments, L is —O—(CR₃R₄)₁₋₄—, wherein each R₃ and R₄ are independently selected from H, D, halogen, C₁₋₄ alkyl (preferably L is —O—CH₂CH₂—, or —O—CH₂—).

In embodiments L is —NR₂—(CR₃R₄)₁₋₄—, wherein each R₃ and R₄ are independently selected from H, D, halogen, C₁₋₄ alkyl (preferably L is —NHCH₂CH₂—, or —NHCH₂—).

In embodiments, L is —O—(CH₂)₁₋₄—.

In some of any of the embodiments described herein, the compound of Formula I is a compound of Formula IIa or IIb:

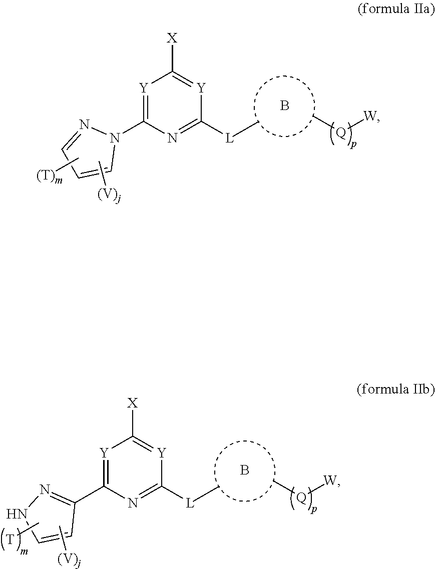

or a pharmaceutically acceptable salt thereof.

In some of any of the embodiments described herein, Ring B is a 5-membered aromatic ring having 1-3 heteroatoms selected from N, O, or S. Preferably, Ring B comprises 2-3 heteroatoms. Preferably, Ring B comprises 2 heteroatoms. Preferably, Ring B comprises 3 heteroatoms. Preferably, the heteroatom is N.

In embodiments, ring B can be optionally substituted. In embodiments, the substituents are independently selected from halogen, —OH, a C₁₋₄ alkyl, and a C₁₋₄ alkoxy.

In some of any of the embodiments described herein, Ring B is selected from:

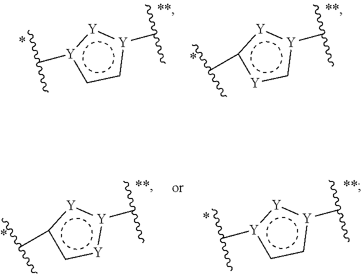

wherein [symbol] is where ring B binds to L and wherein [symbol] is where ring B binds to “(Q)ₚ-W”.

In some of any of the embodiments described herein, Ring B is selected from:

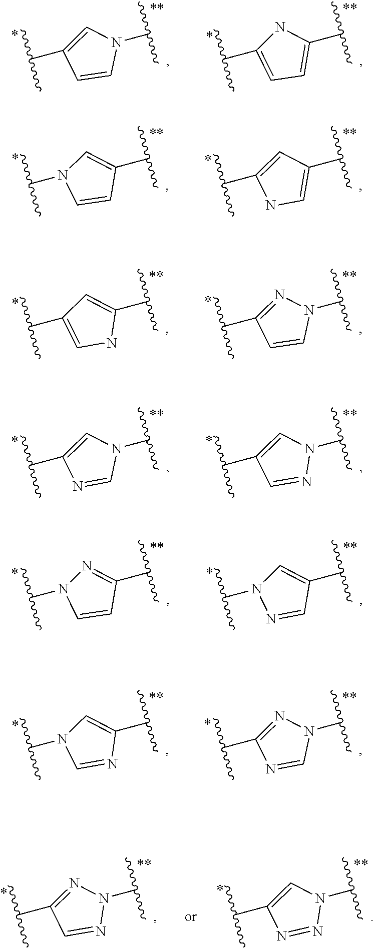

In some of any of the embodiments described herein, Ring B is selected from:

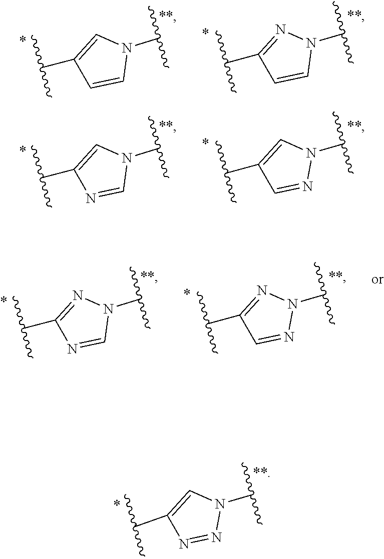

In some of any of the embodiments described herein, Ring B is selected from:

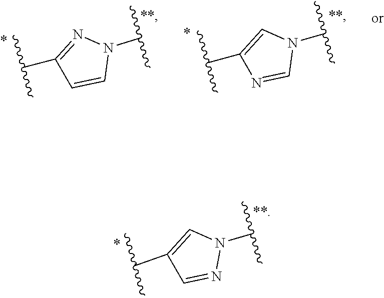 [SMILES: *c1ccn(*)n1 ; *c1cn(*)cn1 ; *c1cnn(*)c1]

In some of any of the embodiments described herein, Ring B is:

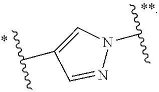 [SMILES: *c1cnn(*)c1]

In some of any of the embodiments described herein, the compound of Formula I is a compound of Formula III:

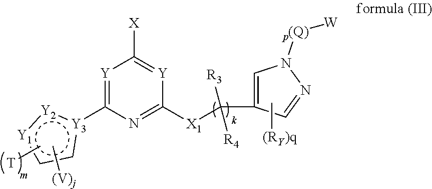

or a pharmaceutically acceptable salt thereof, wherein X₁ is —NR₂—, or —O-(preferably, —O—); k is 1, 2, 3, or 4; each R₃ and R₄ are independently selected from H, D, halogen, C₁₋₄ alkyl; p is 0, or p is 1, Q is —S(O)₂—; Y₁, Y₂, Y₃, are each independently selected from CR₁, N, O, or S(preferably from CR₁, N), provided that two of Y₁, Y₂ and Y₃ are N; R_{Y} is halogen, nitrile, alkyl, hydroxyl, or alkoxy; or R_{Y} and one of the adjacent R₃ and R₄ with the atoms to which they are attached form a bicyclic ring system; q is 0, 1, or 2.

In some of any of the embodiments described herein, p is 0.

In some of any of the embodiments described herein, p is 1, Q is —S(O)₂—.

In some of any of the embodiments described herein, the compound of formula I is a compound of formula IV:

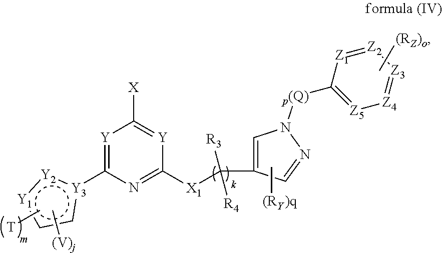

or a pharmaceutically acceptable salt thereof; wherein Z₁, Z₂, Z₃, Z₄, and Z₅ are each independently selected from CR₁, N, O, or S(preferably from CR₁, N), provided at least three of Z₁, Z₂, Z₃, Z₄, and Z₅ are CR₁; and R_{Z} is halogen, nitrile, alkyl, or -G-R_{X}, wherein G is selected from —C(O)—, —CR₃R₄—, —O—, —S(O)₂—, —NR₂—, —C(O)O—, —OC(O)—, —C(O)NR₂—, —NR₂C(O)—, —S(O)—, —S(O)(NR₂)—, —S(O)₂ (NR₂)—, —OC(O)NR₂—, —NR₂C(O)O—; Rₓ is H or a C₁-C₄ alkyl; o is 0, 1, 2, 3, 4, or 5.

In some of any of the embodiments described herein, the compound of formula I is a compound of formula Va or Vb:

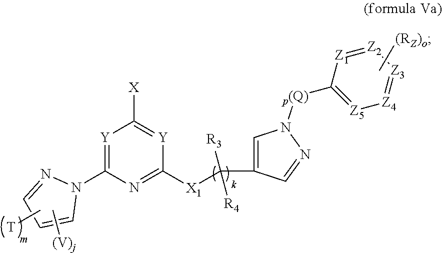

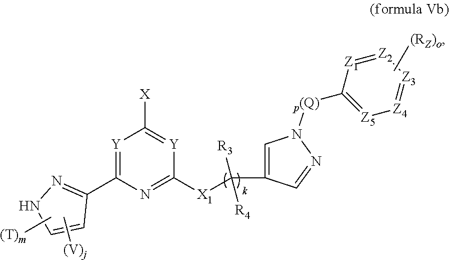

or a pharmaceutically acceptable salt thereof.

In some of any of the embodiments described herein, wherein the compound of formula I is a compound of formula VIa or VIb:

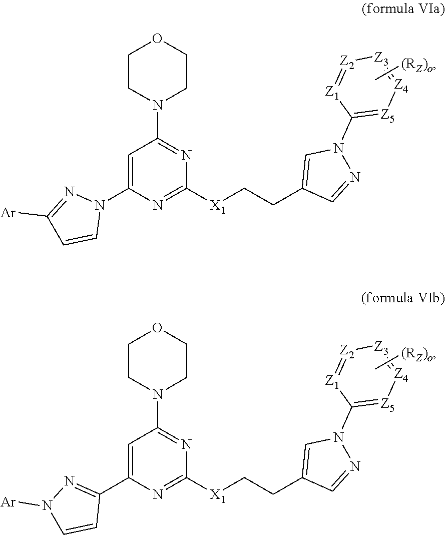

or a pharmaceutically acceptable salt thereof.

In some of any of the embodiments described herein, wherein the compound of formula I is a compound of formula VIc or VId:

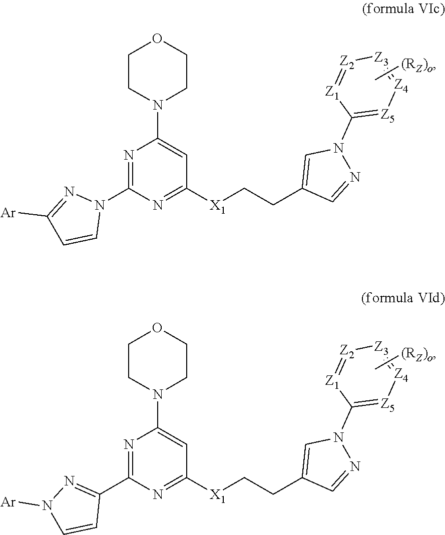

or a pharmaceutically acceptable salt thereof.

In some of any of the embodiments described herein, wherein the compound of formula I is of formula VIIa or VIIb:

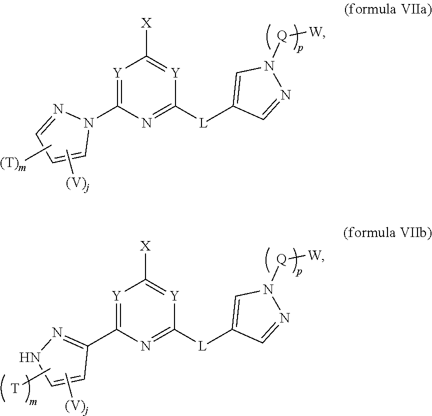

or a pharmaceutically acceptable salt thereof.

In some of any of the embodiments described herein, wherein the compound of formula I is a compound of formula VIIIa, VIIIb, VIIIc, or VIIId:

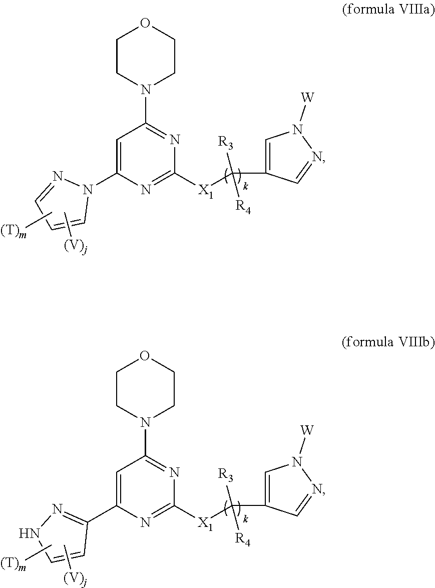

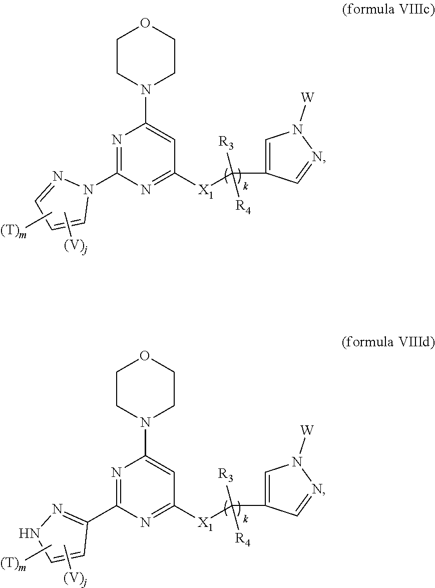

or a pharmaceutically acceptable salt thereof; wherein X₁ is —NR₂—, or —O-(preferably, —O—); k is 1, 2, 3, or 4; each R₃ and R₄ are independently selected from H, D, halogen, C₁₋₄ alkyl; and wherein W is substituted or unsubstituted phenyl, substituted or unsubstituted pyridine, or substituted or unsubstituted pyrimidine.

In some of any of the embodiments described herein, wherein the compound of formula I is a compound of formula IXa, IXb, IXc, or IXd:

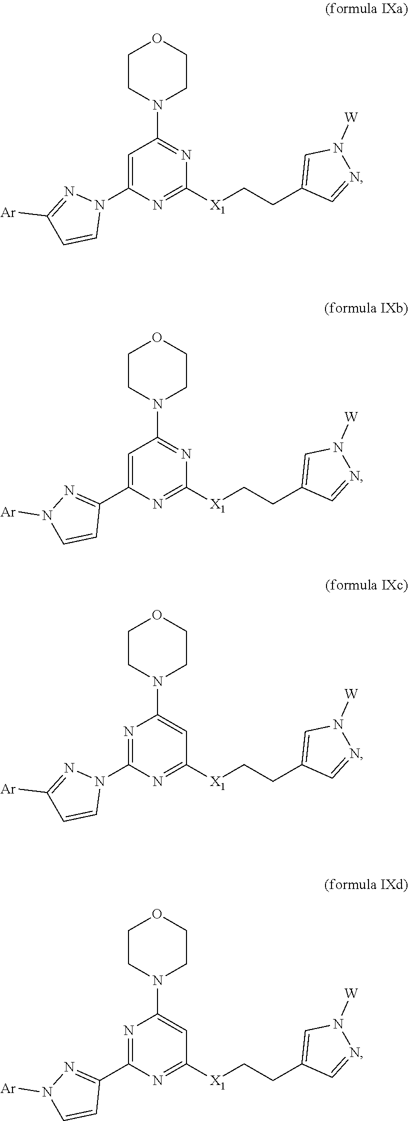

or a pharmaceutically acceptable salt thereof; wherein X₁ is —NR₂—or —O-(preferably, —O—), and Ar is a substituted or unsubstituted aryl ring, preferably a substituted or unsubstituted phenyl or a substituted or unsubstituted pyridinyl, wherein preferred substituents are selected from fluorine, chlorine, and methyl; and wherein W is substituted phenyl, substituted pyridine, or substituted pyrimidine; wherein the phenyl, pyridine, or pyrimidine is substituted with 1-5 substituents selected from halogen, nitrile, —S(O)₂—R_{X}, wherein R_{X} is H or a C₁-C₄ alkyl (preferably, methyl).

Representative compounds of the present invention include compounds listed in Table 1.

TABLE 1

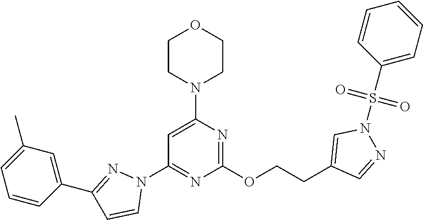 [SMILES: Cc1cccc(-c2ccn(-c3cc(N4CCOCC4)nc(OCCc4cnn(S(=O)(=O)c5ccccc5)c4)n3)n2)c1]

Example 1

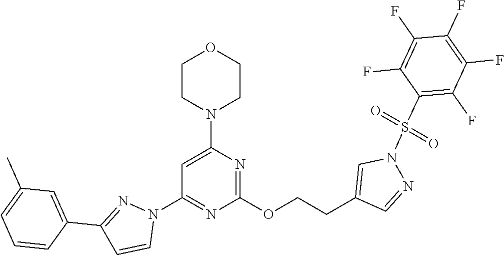 [SMILES: Cc1cccc(-c2ccn(-c3cc(N4CCOCC4)nc(OCCc4cnn(S(=O)(=O)c5c(F)c(F)c(F)c(F)c5F)c4)n3)n2)c1]

Example 2

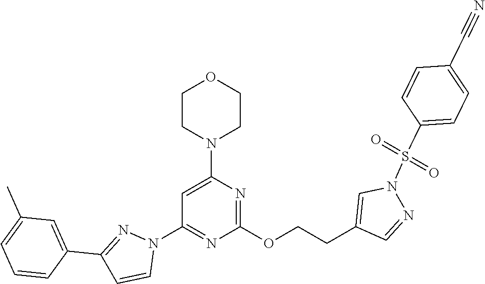 [SMILES: Cc1cccc(-c2ccn(-c3cc(N4CCOCC4)nc(OCCc4cnn(S(=O)(=O)c5ccc(C#N)cc5)c4)n3)n2)c1]

Example 3

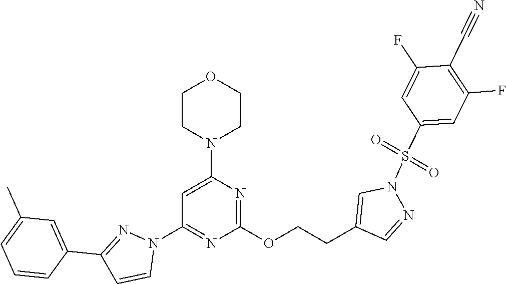 [SMILES: Cc1cccc(-c2ccn(-c3cc(N4CCOCC4)nc(OCCc4cnn(S(=O)(=O)c5cc(F)c(C#N)c(F)c5)c4)n3)n2)c1]

Example 4

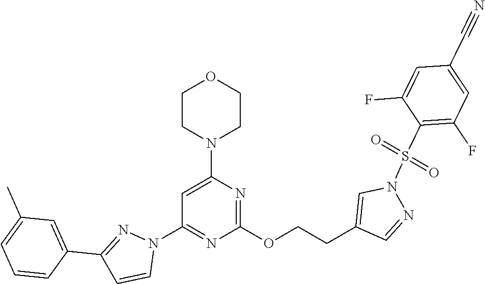 [SMILES: Cc1cccc(-c2ccn(-c3cc(N4CCOCC4)nc(OCCc4cnn(S(=O)(=O)c5c(F)cc(C#N)cc5F)c4)n3)n2)c1]

Example 5

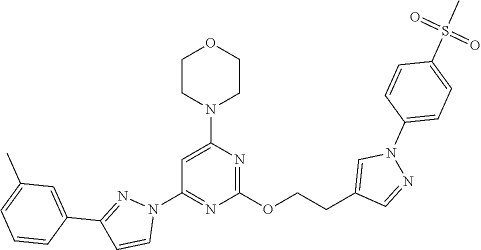 [SMILES: Cc1cccc(-c2ccn(-c3cc(N4CCOCC4)nc(OCCc4cnn(-c5ccc(S(C)(=O)=O)cc5)c4)n3)n2)c1]

Example 6

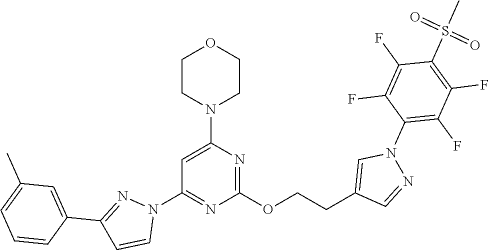 [SMILES: Cc1cccc(-c2ccn(-c3cc(N4CCOCC4)nc(OCCc4cnn(-c5c(F)c(F)c(S(C)(=O)=O)c(F)c5F)c4)n3)n2)c1]

Example 7

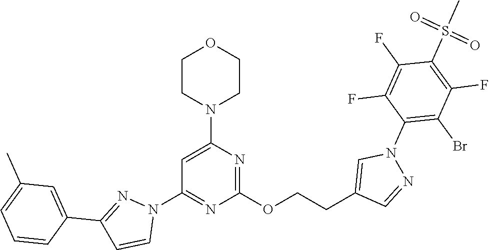 [SMILES: Cc1cccc(-c2ccn(-c3cc(N4CCOCC4)nc(OCCc4cnn(-c5c(F)c(F)c(S(C)(=O)=O)c(F)c5Br)c4)n3)n2)c1]

Example 8

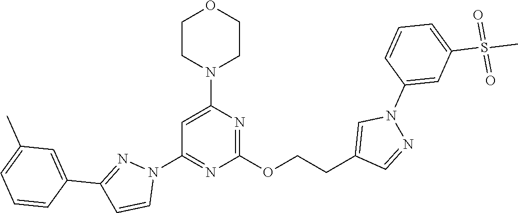 [SMILES: Cc1cccc(-c2ccn(-c3cc(N4CCOCC4)nc(OCCc4cnn(-c5cccc(S(C)(=O)=O)c5)c4)n3)n2)c1]

Example 9

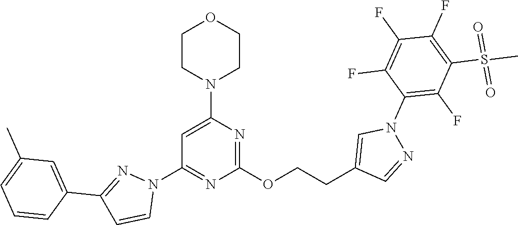 [SMILES: Cc1cccc(-c2ccn(-c3cc(N4CCOCC4)nc(OCCc4cnn(-c5c(F)c(F)c(F)c(S(C)(=O)=O)c5F)c4)n3)n2)c1]

Example 10

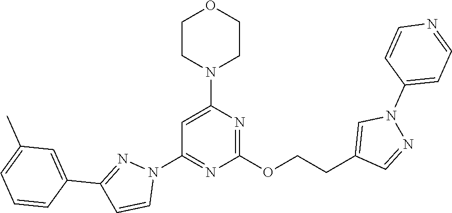 [SMILES: Cc1cccc(-c2ccn(-c3cc(N4CCOCC4)nc(OCCc4cnn(-c5ccncc5)c4)n3)n2)c1]

Example 11

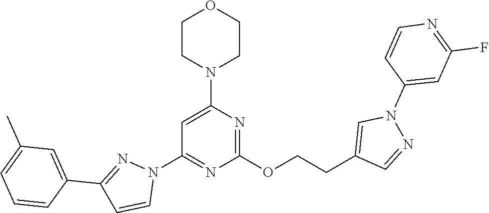 [SMILES: Cc1cccc(-c2ccn(-c3cc(N4CCOCC4)nc(OCCc4cnn(-c5ccnc(F)c5)c4)n3)n2)c1]

Example 12

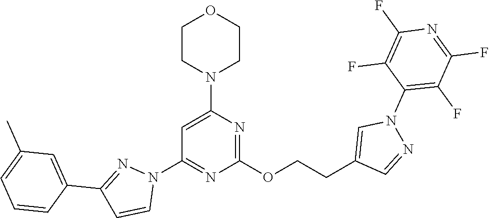 [SMILES: Cc1cccc(-c2ccn(-c3cc(N4CCOCC4)nc(OCCc4cnn(-c5c(F)c(F)nc(F)c5F)c4)n3)n2)c1]

Example 13

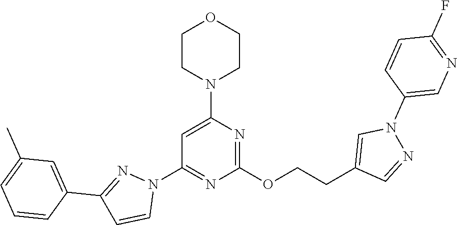 [SMILES: Cc1cccc(-c2ccn(-c3cc(N4CCOCC4)nc(OCCc4cnn(-c5ccc(F)nc5)c4)n3)n2)c1]

Example 14

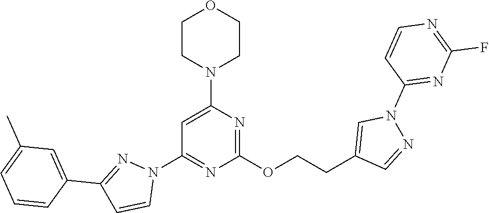 [SMILES: Cc1cccc(-c2ccn(-c3cc(N4CCOCC4)nc(OCCc4cnn(-c5ccnc(F)n5)c4)n3)n2)c1]

Example 15

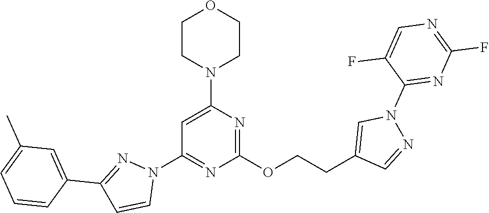 [SMILES: Cc1cccc(-c2ccn(-c3cc(N4CCOCC4)nc(OCCc4cnn(-c5nc(F)ncc5F)c4)n3)n2)c1]

Example 16

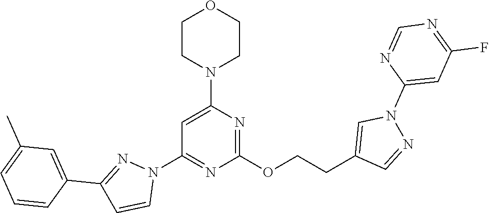 [SMILES: Cc1cccc(-c2ccn(-c3cc(N4CCOCC4)nc(OCCc4cnn(-c5cc(F)ncn5)c4)n3)n2)c1]

Example 17

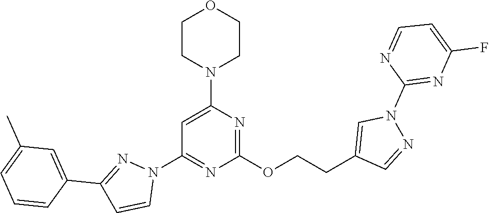 [SMILES: Cc1cccc(-c2ccn(-c3cc(N4CCOCC4)nc(OCCc4cnn(-c5nccc(F)n5)c4)n3)n2)c1]

Example 18

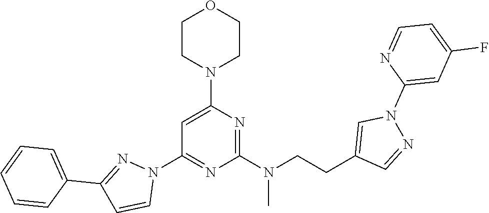 [SMILES: CN(CCc1cnn(-c2cc(F)ccn2)c1)c1nc(N2CCOCC2)cc(-n2ccc(-c3ccccc3)n2)n1]

Example 19

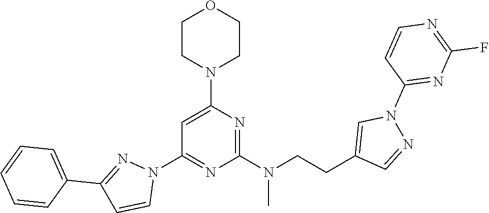 [SMILES: CN(CCc1cnn(-c2ccnc(F)n2)c1)c1nc(N2CCOCC2)cc(-n2ccc(-c3ccccc3)n2)n1]

Example 20

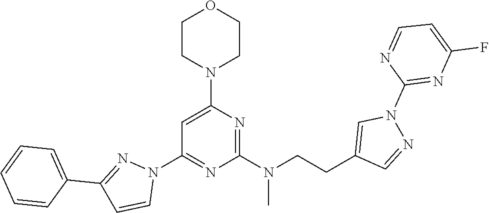 [SMILES: CN(CCc1cnn(-c2nccc(F)n2)c1)c1nc(N2CCOCC2)cc(-n2ccc(-c3ccccc3)n2)n1]

Example 21

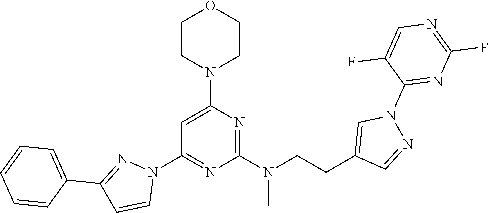 [SMILES: CN(CCc1cnn(-c2nc(F)ncc2F)c1)c1nc(N2CCOCC2)cc(-n2ccc(-c3ccccc3)n2)n1]

Example 22

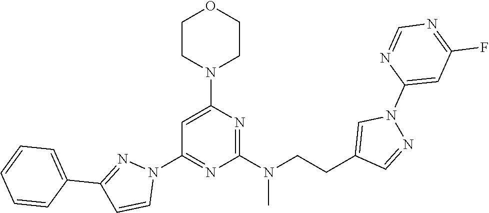 [SMILES: CN(CCc1cnn(-c2cc(F)ncn2)c1)c1nc(N2CCOCC2)cc(-n2ccc(-c3ccccc3)n2)n1]

Example 23

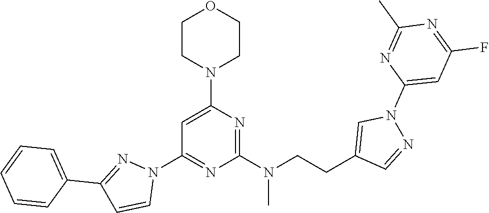 [SMILES: Cc1nc(F)cc(-n2cc(CCN(C)c3nc(N4CCOCC4)cc(-n4ccc(-c5ccccc5)n4)n3)cn2)n1]

Example 24

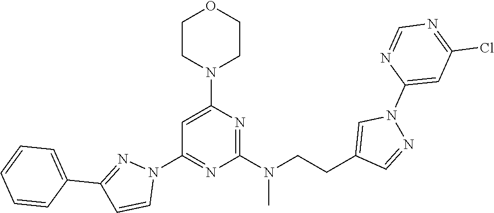 [SMILES: CN(CCc1cnn(-c2cc(Cl)ncn2)c1)c1nc(N2CCOCC2)cc(-n2ccc(-c3ccccc3)n2)n1]

Example 25

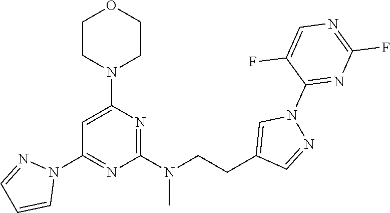 [SMILES: CN(CCc1cnn(-c2nc(F)ncc2F)c1)c1nc(N2CCOCC2)cc(-n2cccn2)n1]

Example 26

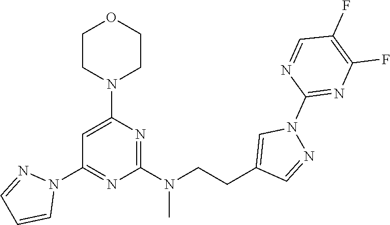 [SMILES: CN(CCc1cnn(-c2ncc(F)c(F)n2)c1)c1nc(N2CCOCC2)cc(-n2cccn2)n1]

Example 27

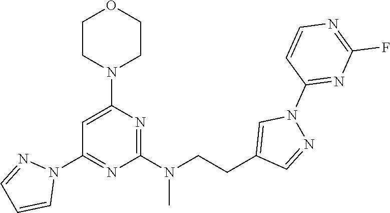 [SMILES: CN(CCc1cnn(-c2ccnc(F)n2)c1)c1nc(N2CCOCC2)cc(-n2cccn2)n1]

Example 28

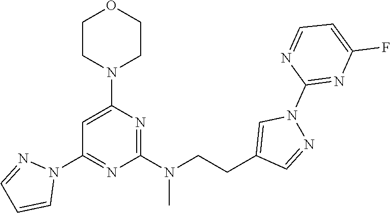 [SMILES: CN(CCc1cnn(-c2nccc(F)n2)c1)c1nc(N2CCOCC2)cc(-n2cccn2)n1]

Example 29

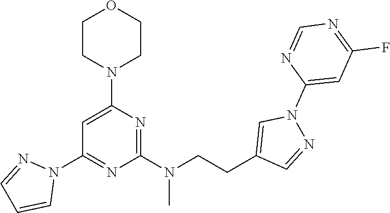 [SMILES: CN(CCc1cnn(-c2cc(F)ncn2)c1)c1nc(N2CCOCC2)cc(-n2cccn2)n1]

Example 30

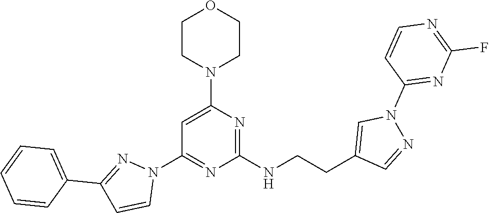 [SMILES: Fc1nccc(-n2cc(CCNc3nc(N4CCOCC4)cc(-n4ccc(-c5ccccc5)n4)n3)cn2)n1]

Example 31

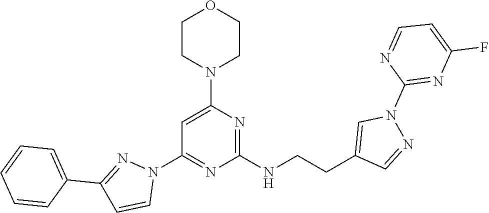 [SMILES: Fc1ccnc(-n2cc(CCNc3nc(N4CCOCC4)cc(-n4ccc(-c5ccccc5)n4)n3)cn2)n1]

Example 32

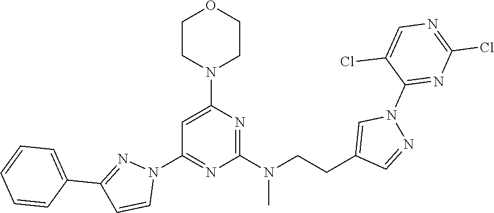 [SMILES: CN(CCc1cnn(-c2nc(Cl)ncc2Cl)c1)c1nc(N2CCOCC2)cc(-n2ccc(-c3ccccc3)n2)n1]

Example 33

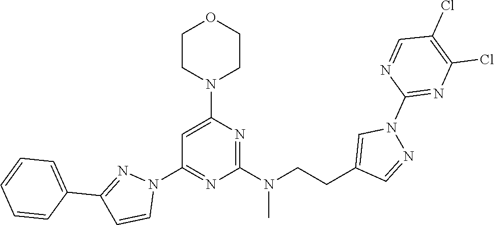 [SMILES: CN(CCc1cnn(-c2ncc(Cl)c(Cl)n2)c1)c1nc(N2CCOCC2)cc(-n2ccc(-c3ccccc3)n2)n1]

Example 34

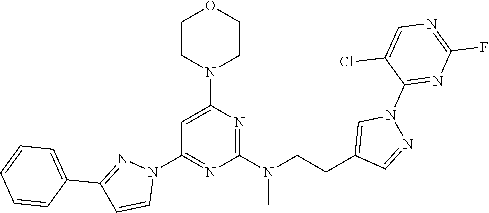 [SMILES: CN(CCc1cnn(-c2nc(F)ncc2Cl)c1)c1nc(N2CCOCC2)cc(-n2ccc(-c3ccccc3)n2)n1]

Example 35

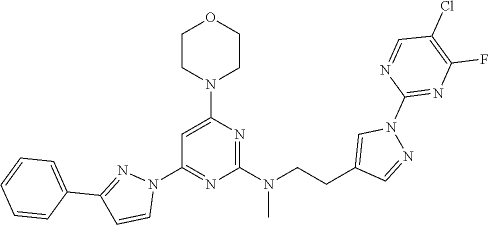 [SMILES: CN(CCc1cnn(-c2ncc(Cl)c(F)n2)c1)c1nc(N2CCOCC2)cc(-n2ccc(-c3ccccc3)n2)n1]

Example 36

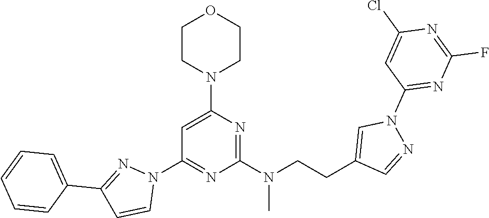 [SMILES: CN(CCc1cnn(-c2cc(Cl)nc(F)n2)c1)c1nc(N2CCOCC2)cc(-n2ccc(-c3ccccc3)n2)n1]

Example 37

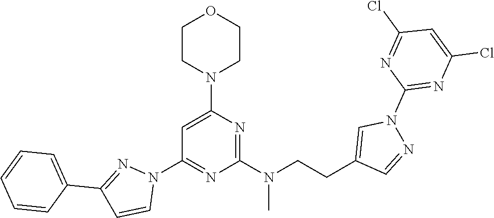 [SMILES: CN(CCc1cnn(-c2nc(Cl)cc(Cl)n2)c1)c1nc(N2CCOCC2)cc(-n2ccc(-c3ccccc3)n2)n1]

Example 38

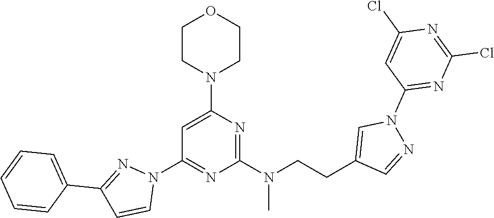 [SMILES: CN(CCc1cnn(-c2cc(Cl)nc(Cl)n2)c1)c1nc(N2CCOCC2)cc(-n2ccc(-c3ccccc3)n2)n1]

Example 39

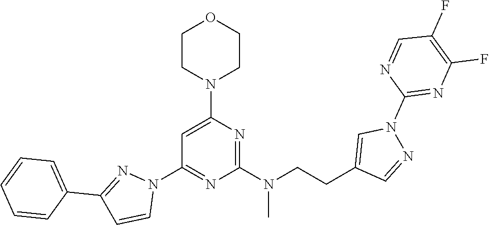 [SMILES: CN(CCc1cnn(-c2ncc(F)c(F)n2)c1)c1nc(N2CCOCC2)cc(-n2ccc(-c3ccccc3)n2)n1]

Example 40

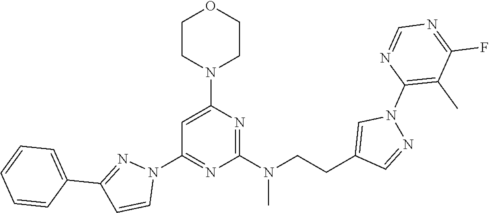 [SMILES: Cc1c(F)ncnc1-n1cc(CCN(C)c2nc(N3CCOCC3)cc(-n3ccc(-c4ccccc4)n3)n2)cn1]

Example 41

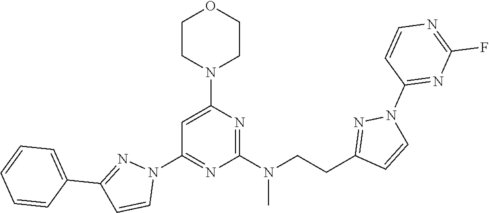 [SMILES: CN(CCc1ccn(-c2ccnc(F)n2)n1)c1nc(N2CCOCC2)cc(-n2ccc(-c3ccccc3)n2)n1]

Example 42

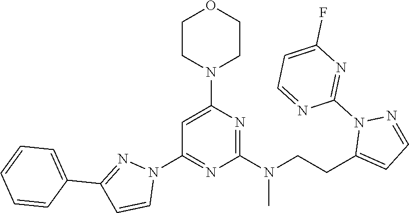 [SMILES: CN(CCc1ccnn1-c1nccc(F)n1)c1nc(N2CCOCC2)cc(-n2ccc(-c3ccccc3)n2)n1]

Example 43

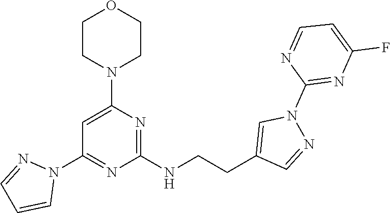 [SMILES: Fc1ccnc(-n2cc(CCNc3nc(N4CCOCC4)cc(-n4cccn4)n3)cn2)n1]

Example 44

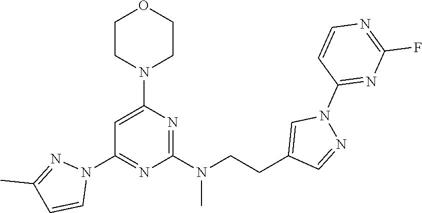 [SMILES: Cc1ccn(-c2cc(N3CCOCC3)nc(N(C)CCc3cnn(-c4ccnc(F)n4)c3)n2)n1]

Example 45

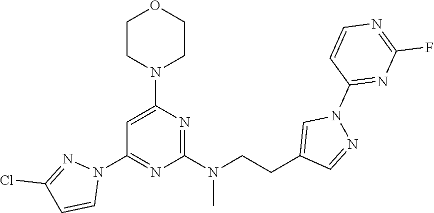 [SMILES: CN(CCc1cnn(-c2ccnc(F)n2)c1)c1nc(N2CCOCC2)cc(-n2ccc(Cl)n2)n1]

Example 46

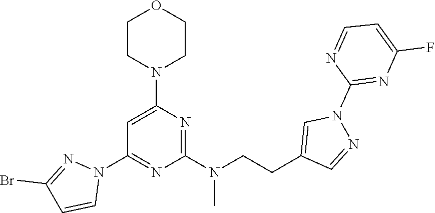 [SMILES: CN(CCc1cnn(-c2nccc(F)n2)c1)c1nc(N2CCOCC2)cc(-n2ccc(Br)n2)n1]

Example 47

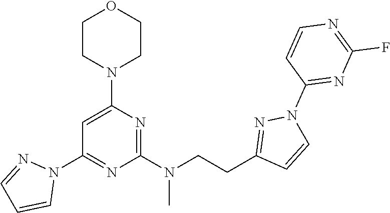 [SMILES: CN(CCc1ccn(-c2ccnc(F)n2)n1)c1nc(N2CCOCC2)cc(-n2cccn2)n1]

Example 48

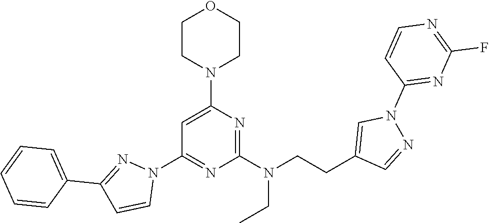 [SMILES: CCN(CCc1cnn(-c2ccnc(F)n2)c1)c1nc(N2CCOCC2)cc(-n2ccc(-c3ccccc3)n2)n1]

Example 49

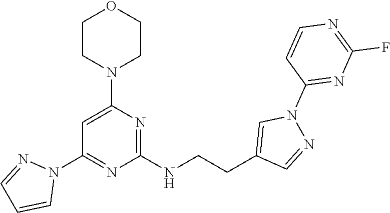 [SMILES: Fc1nccc(-n2cc(CCNc3nc(N4CCOCC4)cc(-n4cccn4)n3)cn2)n1]

Example 50

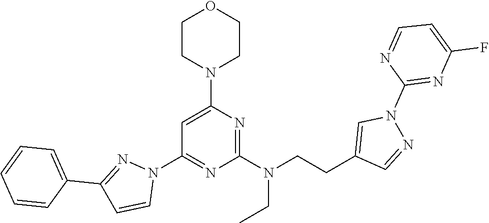 [SMILES: CCN(CCc1cnn(-c2nccc(F)n2)c1)c1nc(N2CCOCC2)cc(-n2ccc(-c3ccccc3)n2)n1]

Example 51

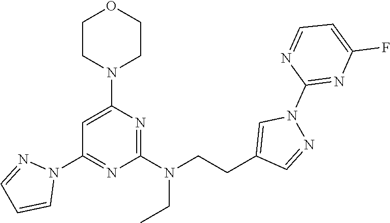 [SMILES: CCN(CCc1cnn(-c2nccc(F)n2)c1)c1nc(N2CCOCC2)cc(-n2cccn2)n1]

Example 52

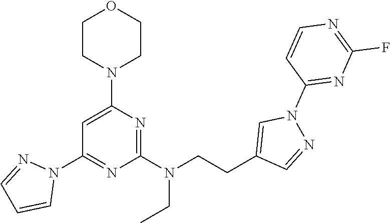 [SMILES: CCN(CCc1cnn(-c2ccnc(F)n2)c1)c1nc(N2CCOCC2)cc(-n2cccn2)n1]

Example 53

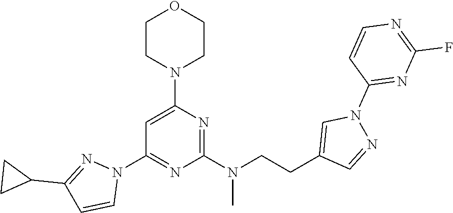 [SMILES: CN(CCc1cnn(-c2ccnc(F)n2)c1)c1nc(N2CCOCC2)cc(-n2ccc(C3CC3)n2)n1]

Example 54

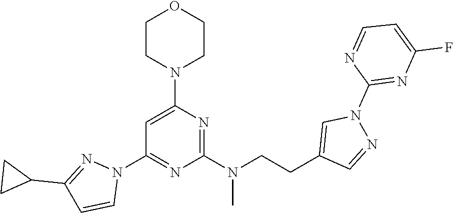 [SMILES: CN(CCc1cnn(-c2nccc(F)n2)c1)c1nc(N2CCOCC2)cc(-n2ccc(C3CC3)n2)n1]

Example 55

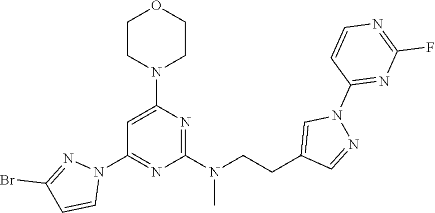 [SMILES: CN(CCc1cnn(-c2ccnc(F)n2)c1)c1nc(N2CCOCC2)cc(-n2ccc(Br)n2)n1]

Example 56

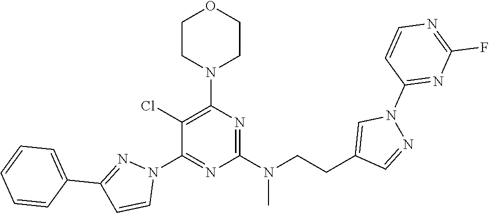 [SMILES: CN(CCc1cnn(-c2ccnc(F)n2)c1)c1nc(N2CCOCC2)c(Cl)c(-n2ccc(-c3ccccc3)n2)n1]

Example 57

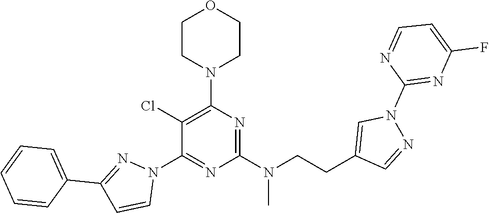 [SMILES: CN(CCc1cnn(-c2nccc(F)n2)c1)c1nc(N2CCOCC2)c(Cl)c(-n2ccc(-c3ccccc3)n2)n1]

Example 58

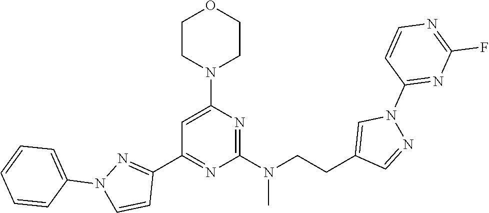 [SMILES: CN(CCc1cnn(-c2ccnc(F)n2)c1)c1nc(-c2ccn(-c3ccccc3)n2)cc(N2CCOCC2)n1]

Example 59

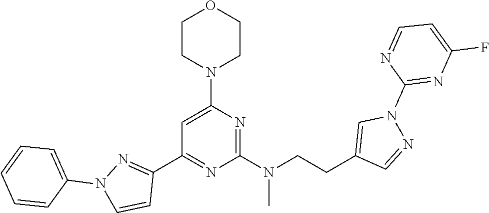 [SMILES: CN(CCc1cnn(-c2nccc(F)n2)c1)c1nc(-c2ccn(-c3ccccc3)n2)cc(N2CCOCC2)n1]

Example 60

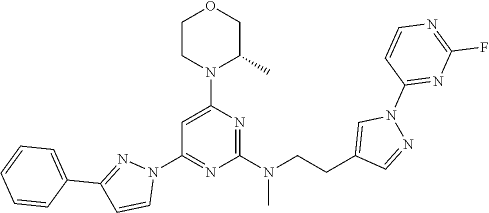 [SMILES: C[C@H]1COCCN1c1cc(-n2ccc(-c3ccccc3)n2)nc(N(C)CCc2cnn(-c3ccnc(F)n3)c2)n1]

Example 61

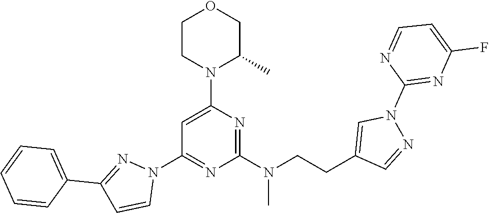 [SMILES: C[C@H]1COCCN1c1cc(-n2ccc(-c3ccccc3)n2)nc(N(C)CCc2cnn(-c3nccc(F)n3)c2)n1]

Example 62

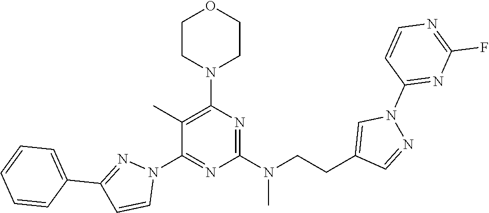 [SMILES: Cc1c(N2CCOCC2)nc(N(C)CCc2cnn(-c3ccnc(F)n3)c2)nc1-n1ccc(-c2ccccc2)n1]

Example 63

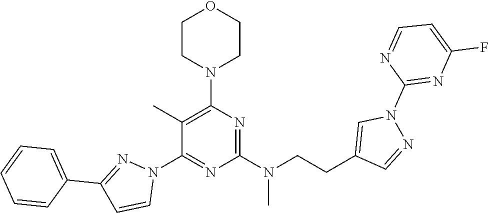 [SMILES: Cc1c(N2CCOCC2)nc(N(C)CCc2cnn(-c3nccc(F)n3)c2)nc1-n1ccc(-c2ccccc2)n1]

Example 64

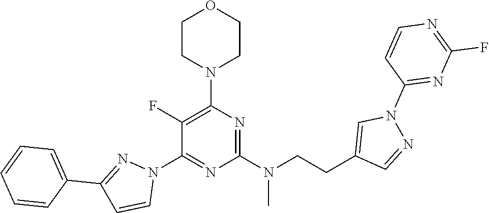 [SMILES: CN(CCc1cnn(-c2ccnc(F)n2)c1)c1nc(N2CCOCC2)c(F)c(-n2ccc(-c3ccccc3)n2)n1]

Example 65

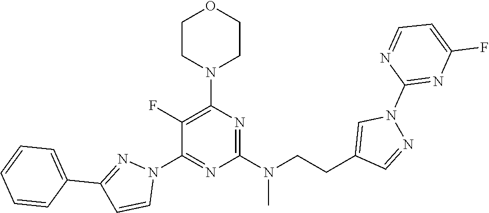 [SMILES: CN(CCc1cnn(-c2nccc(F)n2)c1)c1nc(N2CCOCC2)c(F)c(-n2ccc(-c3ccccc3)n2)n1]

Example 66

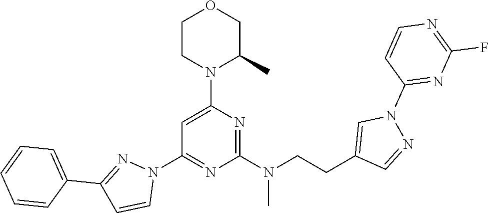 [SMILES: C[C@@H]1COCCN1c1cc(-n2ccc(-c3ccccc3)n2)nc(N(C)CCc2cnn(-c3ccnc(F)n3)c2)n1]

Example 67

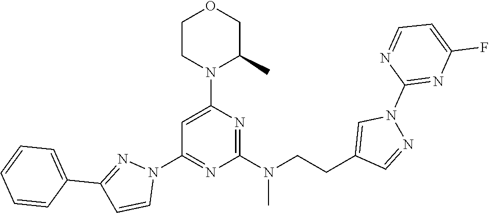 [SMILES: C[C@@H]1COCCN1c1cc(-n2ccc(-c3ccccc3)n2)nc(N(C)CCc2cnn(-c3nccc(F)n3)c2)n1]

Example 68

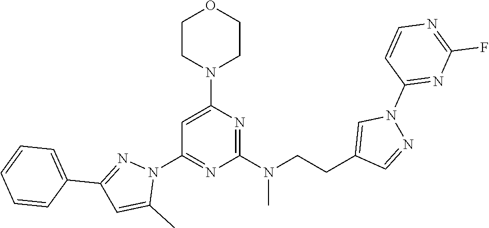 [SMILES: Cc1cc(-c2ccccc2)nn1-c1cc(N2CCOCC2)nc(N(C)CCc2cnn(-c3ccnc(F)n3)c2)n1]

Example 69

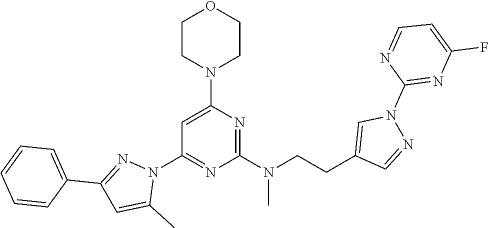 [SMILES: Cc1cc(-c2ccccc2)nn1-c1cc(N2CCOCC2)nc(N(C)CCc2cnn(-c3nccc(F)n3)c2)n1]

Example 70

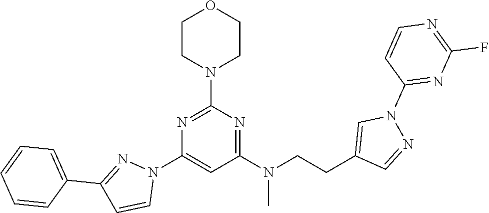 [SMILES: CN(CCc1cnn(-c2ccnc(F)n2)c1)c1cc(-n2ccc(-c3ccccc3)n2)nc(N2CCOCC2)n1]

Example 71

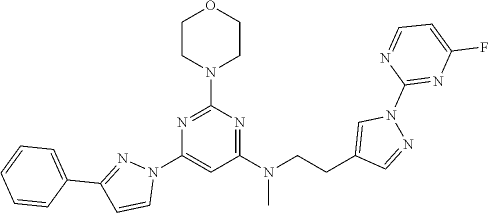 [SMILES: CN(CCc1cnn(-c2nccc(F)n2)c1)c1cc(-n2ccc(-c3ccccc3)n2)nc(N2CCOCC2)n1]

Example 72

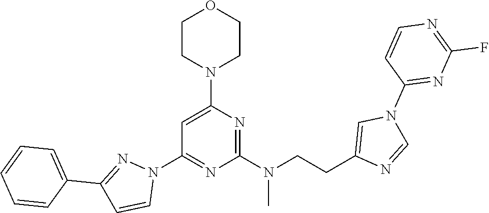 [SMILES: CN(CCc1cn(-c2ccnc(F)n2)cn1)c1nc(N2CCOCC2)cc(-n2ccc(-c3ccccc3)n2)n1]

Example 73

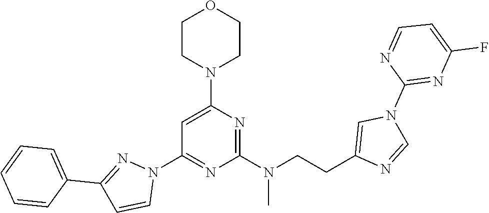 [SMILES: CN(CCc1cn(-c2nccc(F)n2)cn1)c1nc(N2CCOCC2)cc(-n2ccc(-c3ccccc3)n2)n1]

Example 74

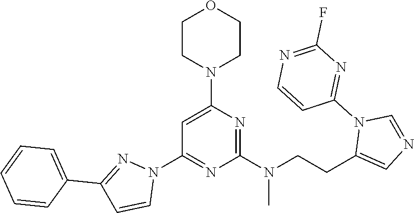 [SMILES: CN(CCc1cncn1-c1ccnc(F)n1)c1nc(N2CCOCC2)cc(-n2ccc(-c3ccccc3)n2)n1]

Example 75

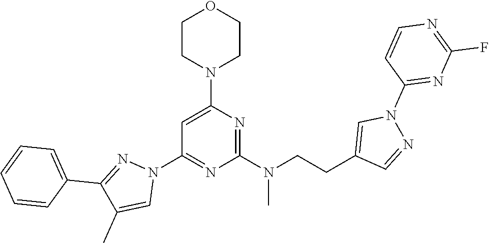 [SMILES: Cc1cn(-c2cc(N3CCOCC3)nc(N(C)CCc3cnn(-c4ccnc(F)n4)c3)n2)nc1-c1ccccc1]

Example 76

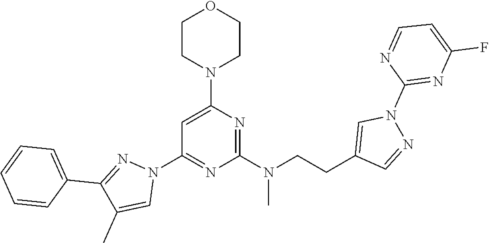 [SMILES: Cc1cn(-c2cc(N3CCOCC3)nc(N(C)CCc3cnn(-c4nccc(F)n4)c3)n2)nc1-c1ccccc1]

Example 77

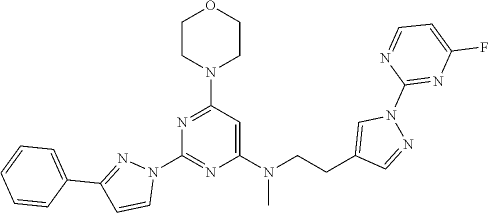 [SMILES: CN(CCc1cnn(-c2nccc(F)n2)c1)c1cc(N2CCOCC2)nc(-n2ccc(-c3ccccc3)n2)n1]

Example 78

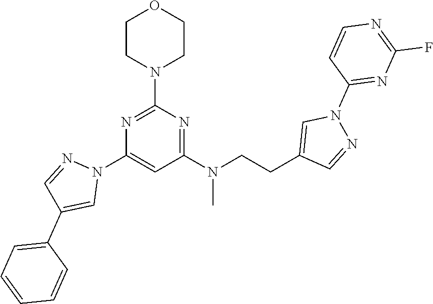 [SMILES: CN(CCc1cnn(-c2ccnc(F)n2)c1)c1cc(-n2cc(-c3ccccc3)cn2)nc(N2CCOCC2)n1]

Example 79

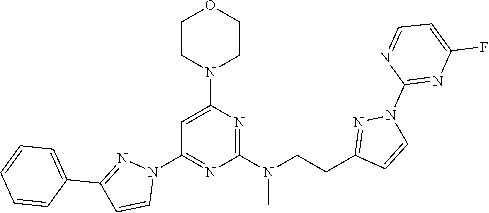 [SMILES: CN(CCc1ccn(-c2nccc(F)n2)n1)c1nc(N2CCOCC2)cc(-n2ccc(-c3ccccc3)n2)n1]

Example 80

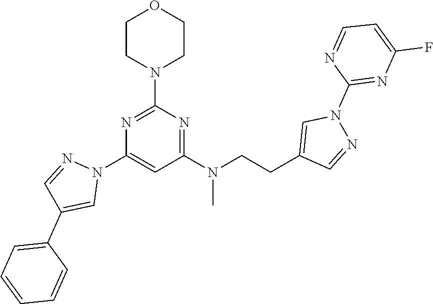 [SMILES: CN(CCc1cnn(-c2nccc(F)n2)c1)c1cc(-n2cc(-c3ccccc3)cn2)nc(N2CCOCC2)n1]

Example 81

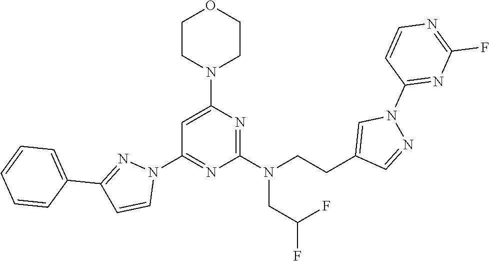 [SMILES: Fc1nccc(-n2cc(CCN(CC(F)F)c3nc(N4CCOCC4)cc(-n4ccc(-c5ccccc5)n4)n3)cn2)n1]

Example 82

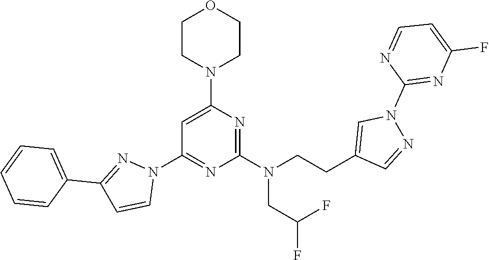 [SMILES: Fc1ccnc(-n2cc(CCN(CC(F)F)c3nc(N4CCOCC4)cc(-n4ccc(-c5ccccc5)n4)n3)cn2)n1]

Example 83

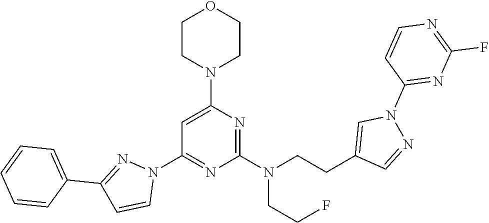 [SMILES: FCCN(CCc1cnn(-c2ccnc(F)n2)c1)c1nc(N2CCOCC2)cc(-n2ccc(-c3ccccc3)n2)n1]

Example 84

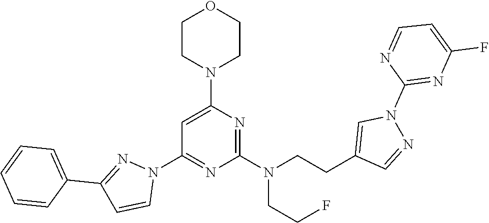 [SMILES: FCCN(CCc1cnn(-c2nccc(F)n2)c1)c1nc(N2CCOCC2)cc(-n2ccc(-c3ccccc3)n2)n1]

Example 85

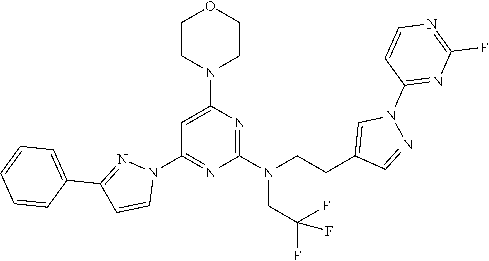 [SMILES: Fc1nccc(-n2cc(CCN(CC(F)(F)F)c3nc(N4CCOCC4)cc(-n4ccc(-c5ccccc5)n4)n3)cn2)n1]

Example 86

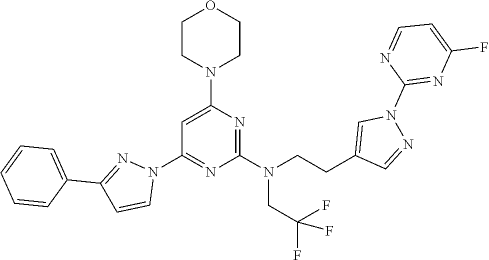 [SMILES: Fc1ccnc(-n2cc(CCN(CC(F)(F)F)c3nc(N4CCOCC4)cc(-n4ccc(-c5ccccc5)n4)n3)cn2)n1]

Example 87

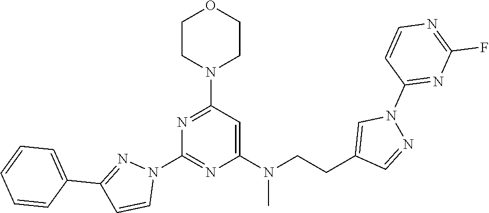 [SMILES: CN(CCc1cnn(-c2ccnc(F)n2)c1)c1cc(N2CCOCC2)nc(-n2ccc(-c3ccccc3)n2)n1]

Example 88

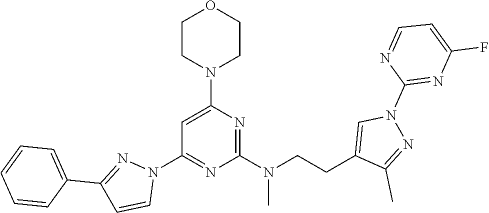 [SMILES: Cc1nn(-c2nccc(F)n2)cc1CCN(C)c1nc(N2CCOCC2)cc(-n2ccc(-c3ccccc3)n2)n1]

Example 89

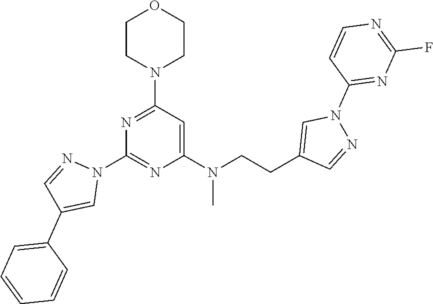 [SMILES: CN(CCc1cnn(-c2ccnc(F)n2)c1)c1cc(N2CCOCC2)nc(-n2cc(-c3ccccc3)cn2)n1]

Example 90

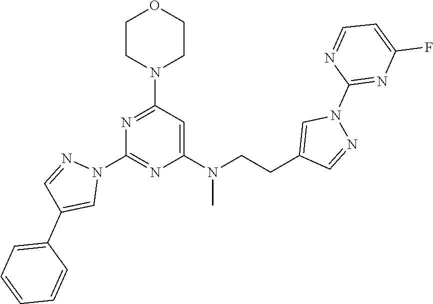 [SMILES: CN(CCc1cnn(-c2nccc(F)n2)c1)c1cc(N2CCOCC2)nc(-n2cc(-c3ccccc3)cn2)n1]

Example 91

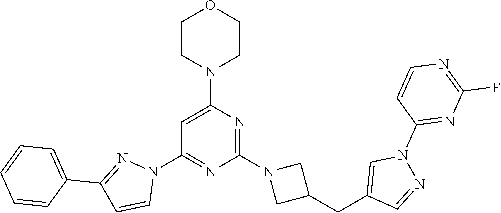 [SMILES: Fc1nccc(-n2cc(CC3CN(c4nc(N5CCOCC5)cc(-n5ccc(-c6ccccc6)n5)n4)C3)cn2)n1]

Example 92

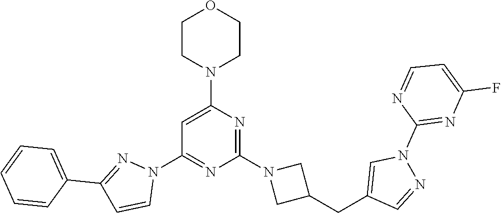 [SMILES: Fc1ccnc(-n2cc(CC3CN(c4nc(N5CCOCC5)cc(-n5ccc(-c6ccccc6)n5)n4)C3)cn2)n1]

Example 93

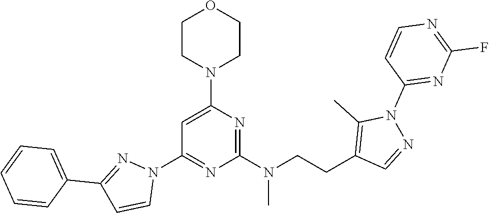 [SMILES: Cc1c(CCN(C)c2nc(N3CCOCC3)cc(-n3ccc(-c4ccccc4)n3)n2)cnn1-c1ccnc(F)n1]

Example 94

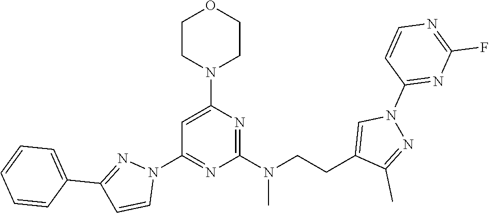 [SMILES: Cc1nn(-c2ccnc(F)n2)cc1CCN(C)c1nc(N2CCOCC2)cc(-n2ccc(-c3ccccc3)n2)n1]

Example 95

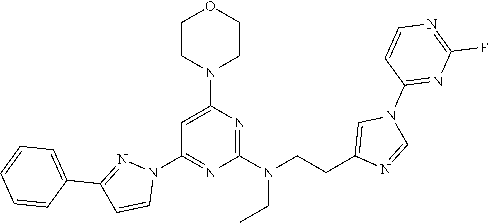 [SMILES: CCN(CCc1cn(-c2ccnc(F)n2)cn1)c1nc(N2CCOCC2)cc(-n2ccc(-c3ccccc3)n2)n1]

Example 96

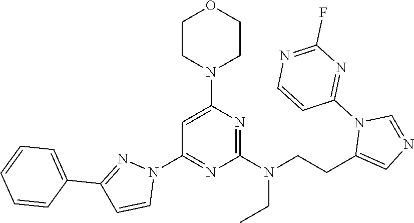 [SMILES: CCN(CCc1cncn1-c1ccnc(F)n1)c1nc(N2CCOCC2)cc(-n2ccc(-c3ccccc3)n2)n1]

Example 97

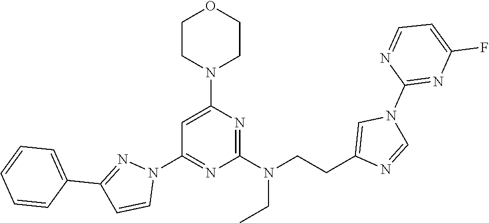 [SMILES: CCN(CCc1cn(-c2nccc(F)n2)cn1)c1nc(N2CCOCC2)cc(-n2ccc(-c3ccccc3)n2)n1]

Example 98

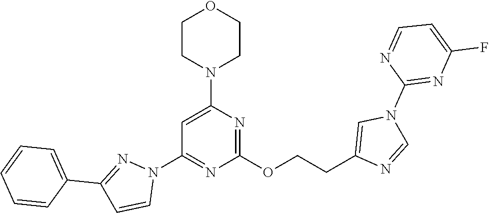 [SMILES: Fc1ccnc(-n2cnc(CCOc3nc(N4CCOCC4)cc(-n4ccc(-c5ccccc5)n4)n3)c2)n1]

Example 99

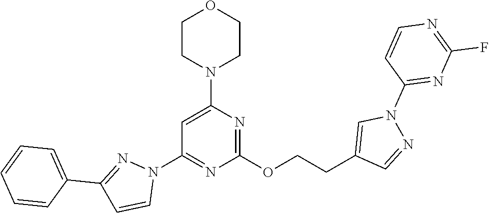 [SMILES: Fc1nccc(-n2cc(CCOc3nc(N4CCOCC4)cc(-n4ccc(-c5ccccc5)n4)n3)cn2)n1]

Example 100

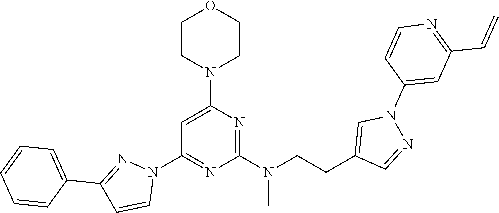 [SMILES: C=Cc1cc(-n2cc(CCN(C)c3nc(N4CCOCC4)cc(-n4ccc(-c5ccccc5)n4)n3)cn2)ccn1]

Example 101

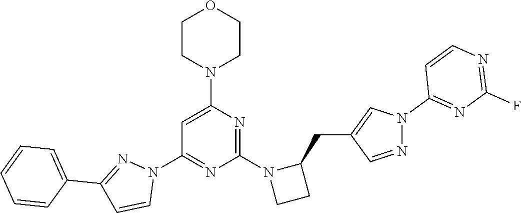 [SMILES: Fc1nccc(-n2cc(C[C@H]3CCN3c3nc(N4CCOCC4)cc(-n4ccc(-c5ccccc5)n4)n3)cn2)n1]

Example 102

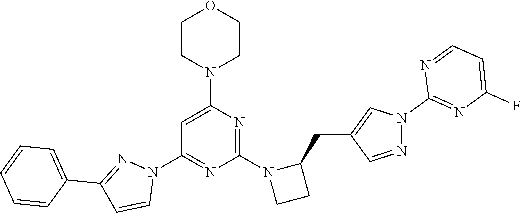 [SMILES: Fc1ccnc(-n2cc(C[C@H]3CCN3c3nc(N4CCOCC4)cc(-n4ccc(-c5ccccc5)n4)n3)cn2)n1]

Example 103

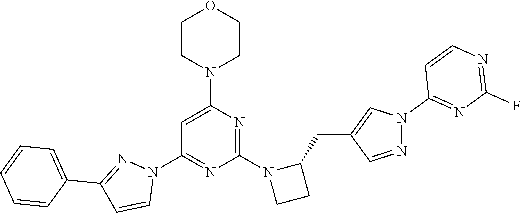 [SMILES: Fc1nccc(-n2cc(C[C@@H]3CCN3c3nc(N4CCOCC4)cc(-n4ccc(-c5ccccc5)n4)n3)cn2)n1]

Example 104

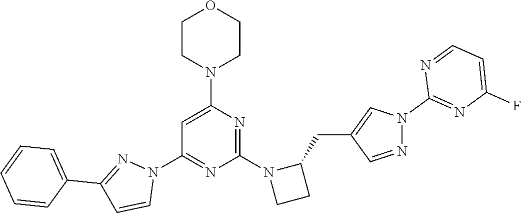 [SMILES: Fc1ccnc(-n2cc(C[C@@H]3CCN3c3nc(N4CCOCC4)cc(-n4ccc(-c5ccccc5)n4)n3)cn2)n1]

Example 105

### Pharmaceutical Composition

One or more compounds according to the invention can be part of a pharmaceutical composition suitable for use in a subject, such as a human. The pharmaceutical composition may comprise at least one pharmaceutically acceptable excipient or carrier.

The pharmaceutical composition may also include at least one additional active agent or therapy. Such agent or therapy may be useful for treating or preventing the disease or condition and does not diminish the activity of the compound according to the invention. Agent(s) useful for treating or preventing the disease or condition include, but are not limited to, small molecules, peptides vaccines, antigens, antibodies, preferably monoclonal antibodies, cytotoxic agents, kinase inhibitors, allergens, antibiotics, siRNA molecules, antisense oligonucleotides, TLR antagonist (e.g. antagonists of TLR3 and/or TLR7 and/or antagonists of TLR8 and/or antagonists of TLR9), chemotherapeutic agents (both traditional chemotherapy and modem targeted therapies), targeted therapeutic agents, activated cells, peptides, proteins, gene therapy vectors, peptide vaccines, protein vaccines, DNA vaccines, adjuvants, and co-stimulatory molecules (e.g. cytokines, chemokines, protein ligands, trans-activating factors, peptides or peptides comprising modified amino acids), or combinations thereof. In embodiments, the agent can be an alkylating agent, an intercalating agent, a tubulin binding agent, a corticosteroid, or any combination of any of the foregoing. Examples of additional active agents include, but are not limited to, ibrutinib, rituximab, doxorubicin, prednisolone, vincristine, velcade, and everolimus, In one embodiment, the at least one additional active agent is a therapeutic agent selected from cyclophosphamide, hydroxydaunorubicin (also referred to as doxorubicin) vincristine, prednisone, prednisolone, and any combination of any of the foregoing.

A pharmaceutical composition can be provided as a dosage unit form, such as an ampoule, a vial, a suppository, a dragee, a tablet, or a capsule. The pharmaceutical compositions can take any suitable form (e.g., liquids, aerosols, solutions, inhalants, mists, sprays; or solids, powders, ointments, pastes, creams, lotions, gels, patches and the like) for administration by any desired route (e.g., pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration, in the form of a sterile aqueous solution or dispersion suitable for administration by either direct injection or by addition to sterile infusion fluids for intravenous infusion, or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase and combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

### Dosage

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of a patient in need thereof.

### Methods of Treatment

The compounds of the present invention are useful as PIKfyve kinase inhibitors. In accordance with the methods described herein, a “subject in need of” is a subject having a disease, disorder or condition, or a subject having an increased risk of developing a disease, disorder or condition relative to the population at large. The subject in need thereof can be one that is “non-responsive” or “refractory” to a currently available therapy for the disease or disorder, for example cancer. In this context, the terms “non-responsive” and “refractory” refer to the subject's response to therapy as not clinically adequate to relieve one or more symptoms associated with the disease or disorder.

In one embodiment, the invention provides a method of treating a subject having a neurological disease. The method comprises administering to the subject an effective amount of a compound of the present invention (or a pharmaceutical composition of the present invention) to the subject.

A “neurological disease” is any disease that causes electrical, genetic, biochemical, or structural abnormalities in the brain, spine, or neurons. For example, a neurological disease may be a neurodegenerative disease. The neurodegenerative disease may result in motor neuron degeneration, for example. The neurological disease may be amyloid lateral sclerosis, Huntington's disease, Alzheimer's disease, or frontotemporal dementia, for example. Further examples of neurological diseases include, but are not limited to, Parkinson's disease, multiple sclerosis, peripheral myopathy, Rasmussen's encephalitis, attention deficit hyperactivity disorder, autism, central pain syndromes, anxiety, and/or depression, for example. Neurodegenerative diseases result in the progressive destruction of neurons that affects neuronal signaling. For example, a neurodegeneration may be amyotrophic lateral sclerosis, Alzheimer's disease, Huntington's disease, Friedreich's ataxia, Lewy body disease, Parkinson's disease, spinal muscle atrophy, primary lateral sclerosis, progressive muscle atrophy, progressive bulbar palsy, and pseudobulbar palsy.

The neurological disease may be one that has neuronal death generated by intracellular aggregates. In certain embodiments, the method includes treating amyotrophic lateral sclerosis (ALS). In certain embodiments, the method includes treating frontotemporal dementia (FTD). In certain embodiments, the method includes treating a neurological disease that is associated with aberrant endosomal trafficking. In certain embodiments, the method includes treating a neurological disease that is associated with aberrant lysosomal trafficking.

In further embodiments, the method includes treating a subject who has a (G₄C₂)ₙ repeat expansion in the C9ORF72 gene. The C9ORF72 gene (also known as chromosome 9 open reading frame 72, FTDALS1, ALSFTD, and FTDALS) is located on the short (p) arm of chromosome 9 at position 21.2. C9ORF72 is a 481 amino acid protein with a molecular mass of 54328 Da, which may undergo post-translational modifications of ubiquitination and phosphorylation. The expression levels of C9ORF72 may be highest in the central nervous system and the protein localizes in the cytoplasm of neurons as well as in presynaptic terminals. C9ORF72 plays a role in endosomal and lysosomal trafficking regulation and has been shown to interact with RAB proteins that are involved in autophagy and endocytic transport. C9ORF72 activates RAB5, a GTPase that mediates early endosomal trafficking. Mutations in C9ORF72 have been associated with ALS and FTD. The G₄C₂ repeat expansion ((G₄C₂)ₙ) in C9ORF72 may be present in subjects suffering from a neurological disease. For example, (G₄C₂)ₙ hexanucleotide expansion is the most common cause of amyotrophic lateral sclerosis (ALS) and frontotemporal dementia (FTD), accounting for about 10% of each worldwide. The (G₄C₂)ₙ hexanucleotide expansion may be located between exons 1a and 1b of C9ORF72. The (G₄C₂)ₙ hexanucleotide expansion may be present in a neurological disease, wherein n is greater than 25, wherein n is greater than 30, wherein n is greater than 35, wherein n is greater than 40, wherein n is greater than 45, wherein n is greater than 50, wherein n is greater than 55, wherein n is greater than 60, wherein n is greater than 65, or wherein n is greater than 70, for example. n may be between 25 and 100, between 29 and 95, between 30 and 90, between 35 and 85, between 40 and 80, between 45 and 75, between 50 and 70, between 55 and 65, or between 55 and 60, for example.

In further embodiments, the subject is haploinsufficient for C9ORF72. In further embodiments, the method includes treating patients who have a 50% or greater reduction in C9ORF72 protein activity. In further embodiments, the method includes a C9ORF72 gene product that comprises a dipeptide repeat resulting from the (G₄C₂)ₙ expansion. In further embodiments, the method includes a gain-of-function or loss of function mutation resulting from the (G₄C₂)ₙ expansion. In further embodiments, the neurological disease is associated with neuronal hyperexcitability.

In one embodiment, the invention provides a method of treating a subject having amyotrophic lateral sclerosis (ALS) comprising administering to the subject an effective amount of a compound of the present invention (or a pharmaceutical composition of the present invention).

In one embodiment, the invention provides a method of treating a subject having frontotemporal dementia (FTD) comprising administering to the subject an effective amount of a compound of the present invention (or a pharmaceutical composition of the present invention).

In one embodiment, the invention provides a method of treating a subject having Alzheimer's disease comprising administering to the subject an effective amount of a compound of the present invention (or a pharmaceutical composition of the present invention).

In one embodiment, the invention provides a method of treating a subject having Parkinson's disease comprising administering to the subject an effective amount of a compound of the present invention (or a pharmaceutical composition of the present invention).

In one embodiment, the invention provides a method of treating a subject having Huntington's disease comprising administering to the subject an effective amount of a compound of the present invention (or a pharmaceutical composition of the present invention).

In one embodiment, the invention provides a method of treating a subject having Charcot-Marie-Tooth disease (CMT) comprising administering to the subject an effective amount of a compound of the present invention (or a pharmaceutical composition of the present invention).

The invention also provides methods for treating a cell proliferative disease, a cancer, or a viral infection in a subject, preferably a human subject, in need of such treatment, by administering an effective amount of a compound of the present invention or a pharmaceutical composition comprising the same, to the subject.

In one embodiment, the invention provides a method of treating a subject having a viral infection. The method comprises administering to the subject an effective amount of a compound of the present invention (or a pharmaceutical composition of the present invention) to the subject. Viral infections include, but are not limited to, infections such as those caused by a virus selected from measles, a coronavirus such as SARS-COV-2, Ebola virus (EboV), Marburg virus (MarV), borna disease, human immunodeficiency virus (HIV), severe acute respiratory system virus (SARS), middle east respiratory syndrome virus (MERS), JC polyomavirus (JC), BK polyomavirus (BK), Herpes Simplex Virus (HSV), Venezuelan equine encephalitis virus (VEEV) and Lymphocytic choriomeningitis virus (LCMV). The viral infection can be caused by any type of virus such as RNA and DNA viruses. In one embodiment, the virus is a coronavirus such as SARS-COV-2. In another embodiment, the virus is Ebola virus. In yet another embodiment, the virus is middle east respiratory syndrome virus (MERS). In yet another embodiment, the virus is JC polyomavirus (JC). In yet another embodiment, the virus is BK polyomavirus (BK). In yet another embodiment, the virus is Herpes Simplex Virus (HSV). In yet another embodiment, the virus is Marburg virus (MarV). In yet another embodiment, the virus is Venezuelan equine encephalitis virus (VEEV). In yet another embodiment, the virus is Lymphocytic choriomeningitis virus (LCMV).

In one embodiment, the invention provides a method of treating a subject having pain. The method comprises administering to the subject an effective amount of a compound of the present invention (or a pharmaceutical composition of the present invention) to the subject. Examples of conditions to be treated include, but are not limited to, nociceptive pain (pain transmitted across intact neuronal pathways), neuropathic pain (pain caused by damage to neural structures), pain from nerve injury (neuromas and neuromas in continuity), pain from neuralgia (pain originating from disease and/or inflammation of nerves), pain from myalgias (pain originating from disease and/or inflammation of muscle), pain associated with painful trigger points, pain from tumors in soft tissues, pain associated with neurotransmitter-dysregulation syndromes (disruptions in quantity/quality of neurotransmitter molecules associated with signal transmission in normal nerves) and pain associated with orthopedic disorders such as conditions of the foot, knee, hip, spine, shoulders, elbow, hand, head and neck.

In one embodiment, the invention provides a method of treating a subject having an inflammatory disease. The method comprises administering to the subject an effective amount of a compound of the present invention (or a pharmaceutical composition of the present invention) to the subject.

“Inflammatory disease” in the context of the present teachings encompasses, without limitation, any disease, as defined herein, resulting from the biological response of vascular tissues to harmful stimuli, including but not limited to such stimuli as pathogens, damaged cells, irritants, antigens and, in the case of autoimmune disease, substances and tissues normally present in the body. Examples of inflammatory disease include rheumatoid arthritis (RA), atherosclerosis, asthma, autoimmune diseases, chronic inflammation, chronic prostatitis, glomerulonephritis, hypersensitivities, inflammatory bowel disease, pelvic inflammatory disease, reperfusion injury, transplant rejection, celiac disease, colitis, irritable bowel syndrome, intestinal hyperplasia, metabolic syndrome, obesity, diabetes, liver disease, hepatic steatosis, fatty liver disease, non-alcoholic fatty liver disease (NAFLD), and non-alcoholic steatohepatitis (NASH), and vasculitis.

In one embodiment, the invention provides a method of treating a subject having an autoimmune disease. The method comprises administering to the subject an effective amount of a compound of the present invention (or a pharmaceutical composition of the present invention) to the subject.

“Autoimmune disease” encompasses any disease, as defined herein, resulting from an immune response against substances and tissues normally present in the body. Examples of suspected or known autoimmune diseases include rheumatoid arthritis, juvenile idiopathic arthritis, seronegative spondyloarthropathies, ankylosing spondylitis, psoriatic arthritis, antiphospholipid antibody syndrome, autoimmune hepatitis, Behçet's disease, bullous pemphigoid, coeliac disease, Crohn's disease, dermatomyositis, Goodpasture's syndrome, Graves' disease, Hashimoto's disease, idiopathic thrombocytopeniaurpura, IgA nephropathy, Kawasaki disease, systemic lupus erythematosus, mixed connective tissue disease, multiple sclerosis, myasthenia gravis, polymyositis, primary biliary cirrhosis, psoriasis, scleroderma, Sjogren's syndrome, ulcerative colitis, vasculitis, Wegener's granulomatosis, temporal arteritis, Takayasu's arteritis, Henoch-Schonlein purpura, leucocytoclastic vasculitis, polyarteritis nodosa, Churg-Strauss Syndrome, and mixed cryoglobulinemic vasculitis.

In one embodiment, the invention provides a method of treating a subject having a lysosomal storage disorder. The method comprises administering to the subject an effective amount of a compound of the present invention (or a pharmaceutical composition of the present invention) to the subject.

Lysosomal storage diseases/disorders (LSDs) includes over forty metabolic disorders, many of which involve genetic defects in various lysosomal hydrolases. Representative lysosomal storage diseases and the associated defective enzymes (in parenthesis) include, but are not limited to, Aspartylglucosaminuria (Aspartylglucosaminidase), Fabry (alpha.-Galactosidase A), Infantile Batten Disease* (CNL1)(Palmitoyl Protein Thioesterase), Classic Late Infantile Batten Disease* (CNL2)(Tripeptidyl Peptidase), Juvenile Batten Disease* (CNL3)(Lysosomal Transmembrane Protein), Batten, other forms* (CNL4-CNL8)(Multiple gene products), Cystinosis (Cysteine transporter), Farber (Acid ceramidase), Fucosidosis (Acid.alpha.-L-fucosidase), Galactosidosialidosis (Protective protein/cathepsin A), Gaucher types 1, 2*, and 3* (Acid.beta.-glucosidase, G.sub.MI gangliosidosis* (Acid.beta.-galactosidase), Hunter* (Iduronate-2-sulfatase), Hurler-Scheie* (alpha.-L-Iduronidase), Krabbe* (Galactocerebrosidase), alpha.-Mannosidosis* (Acid.alpha.-mannosidase), beta.-Mannosidosis* (Acid.beta.-mannosidase), Maroteaux-Lamy (Arylsulfatase B), Metachromatic leukodystrophy* (Arylsulfatase A), Morquio A (N-Acetylgalactosamine-6-sulfate), Morquio B (Acid.beta.-galactosidase), Mucolipidosis II/III* (N-Acetylglucosamine-1-phoshpate transferase) Niemann-Pick A*, B (Acid sphingomyelinase), Niemann-Pick C* (NPC-1), Pompe* (Acid.alpha.-glucosidase), Sandhoff* (.beta.-Hexosaminidase B), Sanfilippo A* (Heparan N-sulfatase), Sanfilippo B* (.alpha.-N-Acetylglucosaminidase), Sanfilippo C* (Acetyl-CoA: alpha.-glucosaminide), Sanfilippo D* (N-Acetylglucosamine-6-sulfate), Schindler Disease* (.alpha.-N-Acetylgalactosaminidase), Schindler-Kanzaki (alpha.—N-Acetylgalactosaminidase), Sialidosis (.alpha.-Neuramidase), Sly* (.beta.-Glucuronidase), Tay-Sachs* (.beta.-Hexosaminidase A), and Wolman* (Acid Lipase), (*CNS involvement).

In one embodiment, the invention provides a method of treating a subject having cancer. The method comprises administering to the subject an effective amount of a compound of the present invention (or a pharmaceutical composition of the present invention) to the subject. In one aspect of the methods described here, the subject in need thereof is a subject having cancer whose cancer is refractory to standard therapy or whose cancer has recurred following standard treatment.

In one embodiment, the cancer is brain cancer, glioma, sarcoma, breast cancer, lung cancer, non-small-cell lung cancer, mesothelioma, appendiceal cancer, genitourinary cancers, renal cell carcinoma, prostate cancer, bladder cancer, testicular cancer, penile cancer, cervical cancer, ovarian cancer, von Hippel Lindau disease, head and neck cancer, gastrointestinal cancer, hepatocellular carcinoma, gallbladder cancer, esophageal cancer, gastric cancer, colorectal cancer, pancreatic cancer, neuroendocrine tumors, thyroid tumor, pituitary tumor, adrenal tumor, hematological malignancy, or leukemia. In another embodiment, the cancer is a melanoma.

In one embodiment the cancer is a lymphoma. In one embodiment, the lymphoma is a B cell lymphoma. In one embodiment, the B cell lymphoma is selected from the group consisting of a Hodgkin's B cell lymphoma and a non-Hodgkin's B cell lymphoma. In one embodiment, the B cell lymphoma is a non-Hodgkin's B cell lymphoma selected from the group consisting of DLBCL, follicular lymphoma, marginal zone lymphoma (MZL) or mucosa associated lymphatic tissue lymphoma (MALT), small cell lymphocytic lymphoma (overlaps with chronic lymphocytic leukemia) and mantle cell lymphoma. In one embodiment, the B cell lymphoma is a non-Hodgkin's B cell lymphoma selected from the group consisting of Burkitt's lymphoma, Primary mediastinal (thymic) large B-cell lymphoma, Lymphoplasmacytic lymphoma, which may manifest as Waldenstrom macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma, Primary central nervous system lymphoma, Primary cutaneous diffuse large B-cell lymphoma, leg type (Primary cutaneous DLBCL, leg type), EBV positive diffuse large B-cell lymphoma of the elderly, Diffuse large B-cell lymphoma associated with inflammation, Intravascular large B-cell lymphoma, ALK-positive large B-cell lymphoma, and Plasmablastic lymphoma.

In a preferred embodiment of any of the methods described herein, the subject is a human subject.

In any of the methods described herein, the method may further comprise also administering an effective amount of a potassium channel activator, an inhibitor of a glutamate receptor (such as the receptor NMDA, AMPA, or kainite)(e.g., AP5, CNQX, and NBQX), or any combination of any of the foregoing.

In any of the methods described herein, the method may further comprise administering an effective amount of one or more pharmaceutically active agents such as immunostimulatory agents, anti-viral agents, antibiotics, anti-fungal agents, anti-parasitic agents, anti-tumor agents, cytokines, chemokines, growth factors, anti-angiogenic factors, chemotherapeutic agents, antibodies and gene silencing agents. Preferably, the pharmaceutically active agent is selected from the group consisting of an immunostimulatory agent, an anti-bacterial agent, an anti-viral agent, an anti-inflammatory agent and an anti-tumor agent. The more than one pharmaceutically active agents may be of the same or different category.

In certain embodiments, the one or more pharmaceutically active agents includes but are not limited to, an antigen, an anti-viral vaccine, an anti-bacterial vaccine, and/or an anti-tumor vaccine, wherein the vaccine can be prophylactic and/or therapeutic.

According to some of any of the embodiments described herein, in any of the methods and uses as described herein, the method may further comprise an additional therapy for treating the disease associated with a tumor (e.g., cancer).

According to some of any of the embodiments described herein, in any of the methods and uses as described herein, the additional therapy for treating the disease associated with a tumor is an anti-cancer therapy.

Suitable anti-cancer therapy includes, for example, chemotherapy, radiotherapy, phototherapy and/or photodynamic therapy, surgery, nutritional therapy, ablative therapy, combined radiotherapy and chemotherapy, brachiotherapy, proton beam therapy, immunotherapy, cellular therapy and photon beam radiosurgical therapy, and any combination of the foregoing.

Chemotherapeutic drugs (e.g., anti-cancer drugs) that may optionally be co-administered to the subject prior to, concomitant with and/or subsequent to one or more compounds as described herein in any of the respective embodiments include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof.

In some embodiments, the anti-cancer therapy comprises immunotherapy, including, for example, checkpoint inhibitors, CAR-T cell therapy, and/or vaccine adjuvants (e.g., interferon or saponin), and immune-adjuvants, such as aluminum salts, organic adjuvants, and genomic material-based adjuvants, such as CpG. Anti-tumor immunity can be further augmented by inhibition of immune suppressor cells.

According to some embodiments, the anti-cancer therapy comprises administration of an anti-cancer immune modulator agent. As used herein, the term “anti-cancer immune modulator agent” refers to an agent capable of eliciting an immune response (e.g. T cell, NK cell) against a cancerous cell. Exemplary anti-cancer immune modulator agents include, but are not limited to, a cancer antigen, a cancer vaccine, an anti-cancer antibody, a cytokine capable of inducing activation and/or proliferation of a T cell and an immune-check point regulator.

Alternatively or additionally, such modulators may be immune stimulators such as immune-check point regulators which are of specific value in the treatment of cancer. As used herein the term “immune-check point regulator” refers to a molecule that modulates the activity of one or more immune-check point proteins in an agonistic or antagonistic manner resulting in activation of an immune cell. As used herein the term “immune-check point protein” refers to a protein that regulates an immune cell activation or function. Immune check-point proteins can be either co-stimulatory proteins (i.e. transmitting a stimulatory signal resulting in activation of an immune cell) or inhibitory proteins (i.e. transmitting an inhibitory signal resulting in suppressing activity of an immune cell). According to some embodiments, the immune check-point protein regulates activation or function of a T cell. Numerous checkpoint proteins are known in the art and include, but not limited to, PD1, PDL-1, B7H2, B7H4, CTLA-4, CD80, CD86, LAG-3, TIM-3, KIR, IDO, CD19, OX40, 4-1BB (CD137), CD27, CD70, CD40, GITR, CD28 and ICOS (CD278).

According to some embodiments, the anti-cancer therapy comprises a surgical procedure, for example, resection or excision of at least a portion of the tumor.

In any of the methods described herein, the compounds (or composition thereof) can be administered by any suitable route, such as an oral, intravenous, or subcutaneous route.

### Examples

### Preparation of the Compounds

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. The following descriptions of synthetic methods are designed to illustrate, but not to limit, general procedures for the preparation of compounds of the present invention.

Compounds of the present invention can be conveniently prepared by a variety of methods familiar to those skilled in the art. The compounds of this invention with each of the formulae described herein may be prepared according to the following procedures from commercially available starting materials or starting materials which can be prepared using literature procedures. These procedures show the preparation of representative compounds of this invention.

The synthetic processes of the invention can tolerate a wide variety of functional groups; therefore various substituted starting materials can be used. The processes generally provide the desired final compound at or near the end of the overall process, although it may be desirable in certain instances to further convert the compound to a pharmaceutically acceptable salt, polymorph, hydrate, solvate or co-crystal thereof.

### Abbreviations

DCM—dichloromethane
EtOAc—ethyl acetate
h—hour(s)
MeOH—methanol
min—minute(s)

LCMS Methods
Method C

| | | |
|---|---|---|
| Mobile Phase | (A) | 2 mM ammonium acetate followed by 0.1% |
| | | formic acid in water |
| | (B) | 0.1% formic acid in acetonitrile |
| Instrument | . | WATERS ACQUITY UPLC H Class with |
| | . | PDA and SQ DETECTOR |
| | . | |
| Column | · | BEH C18 (50 × 2.1 mm) 1.7 μm |
| | . | |
| | . | |
| Flow rate | . | 0.550 mL/min |
| | . | |
| | . | |
| Column oven | . | Ambient |
| temperature | . | |
| | . | |
| Run time | . | 3.0 min |
| | . | |
| | . | |
| Gradient | . | |
| | . | |
| | . | |

| | Flow Rate | | |
|---|---|---|---|
| TIME: | (mL/min) | % A | % B |
| 0.01 | 0.55 | 98 | 2 |
| 0.30 | 0.55 | 98 | 2 |
| 0.60 | 0.55 | 50 | 50 |
| 1.10 | 0.55 | 25 | 75 |
| 2.00 | 0.60 | 0 | 100 |
| 2.70 | 0.60 | 0 | 100 |
| 2.71 | 0.55 | 98 | 2 |
| 3.00 | 0.55 | 98 | 2 |

Method-C Fast

| Mobile Phase | (A) | 2 mM ammonium acetate followed | |
|---|---|---|---|
| | | by 0.1% formic acid in water | |
| | (B) | 0.1% formic acid in acetonitrile | |
| Instrument | . | WATERS ACQUITY UPLC H Class | |
| | . | with PDA and SQ DETECTOR | |
| | . | | |
| Column | . | BEH C18 (50 × 2.1 mm) 1.7 μm | |
| | . | | |
| | . | | |
| Flow rate | . | 0.400 mL/min | |
| | . | | |
| | . | | |
| Column oven | . | Ambient | |
| temperature | . | | |
| | . | | |
| Run time | . | 5.0 min | |
| | . | | |
| | . | | |
| Gradient | . | | |
| | . | | |
| | . | | |
| | Flow Rate | | |
| TIME: | (mL/min) | % A | % B |
| 0.00 | 0.400 | 50 | 50 |
| 1.00 | 0.400 | 10 | 90 |
| 1.50 | 0.400 | 0 | 100 |
| 4.50 | 0.400 | 0 | 100 |
| 4.60 | 0.400 | 50 | 50 |
| 5.00 | 0.400 | 50 | 50 |

Method H

| Mobile Phase | (A) | 5 mM ammonium bicarbonate in water | |
|---|---|---|---|
| | (B) | 100% acetonitrile | |
| Instrument | . | Agilent 1290 Infinity RRLC attached | |
| | . | with Agilent 6120 Mass detector and | |
| | . | Diode array Detector | |
| Column | . | WATERS X-Bridge C18 (50 × 4.6 | |
| | . | mm) 3.5 μm | |
| | . | | |
| Flow rate | . | 1.0 mL/min | |
| | . | | |
| | . | | |
| Column oven | . | Ambient | |
| temperature | . | | |
| | . | | |
| Run time | . | 6.0 min | |
| | . | | |
| | . | | |
| Gradient | . | | |
| | . | | |
| | . | | |
| | Flow Rate | | |
| TIME: | (mL/min) | % A | % B |
| 0.01 | 1.0 | 95 | 5 |
| 2.80 | 1.0 | 15 | 85 |
| 3.50 | 1.0 | 10 | 90 |
| 5.00 | 1.0 | 10 | 90 |
| 5.01 | 1.0 | 95 | 5 |
| 6.00 | 1.0 | 95 | 5 |

Method J

| Mobile Phase | (A) | 2 mM ammonium acetate followed by 0.1% | |
|---|---|---|---|
| | | formic acid in water | |
| | (B) | 0.1% formic acid in acetonitrile | |
| Instrument | . | Agilent 1290 Infinity RRLC attached | |
| | . | with Agilent 6120 Mass detector and | |
| | . | Diode array Detector | |
| Column | . | BEH C18 (50 × 2.1 mm) 1.7 μm | |
| | . | | |
| | . | | |
| Flow rate | . | 0.500 mL/min | |
| | . | | |
| | . | | |
| Column oven | . | Ambient | |
| temperature | . | | |
| | . | | |
| Run time | . | 5.0 min | |
| | . | | |
| | . | | |
| Gradient | . | | |
| | . | | |
| | . | | |
| | Flow Rate | | |
| TIME: | (mL/min) | % A | % B |
| 0.01 | 0.500 | 98 | 2 |
| 0.40 | 0.500 | 98 | 2 |
| 2.50 | 0.500 | 35 | 65 |
| 3.50 | 0.500 | 5 | 95 |
| 4.70 | 0.500 | 5 | 95 |
| 4.71 | 0.500 | 98 | 2 |
| 5.00 | 0.500 | 98 | 2 |

### Example 1:4-(2-(2-(1-(phenylsulfonyl)-1H-pyrazol-4-yl)ethoxy)-6-(3-(m-tolyl)-1H-pyrazol-1-yl)pyrimidin-4-yl)morpholine

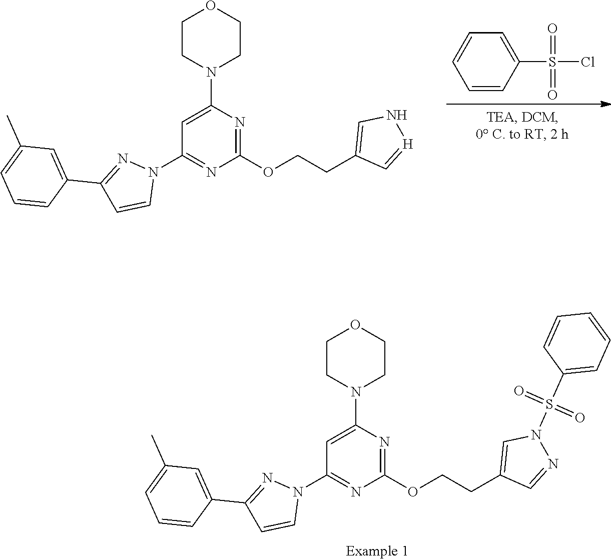

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.232 mmol) in DCM (2 mL) was added triethylamine (0.1 mL, 0.464 mmol) at 0° C. under nitrogen atmosphere. After 5 min benzenesulfonyl chloride (0.082 g, 0.696 mmol) was added at 0° C. The reaction mixture was stirred at room temperature for 2 h. After reaction completion, solvent was removed in vacuum to obtain crude product. The resulting crude was purified by silica gel flash column chromatography. The desired product was eluted at 40% EtOAc in hexanes to obtain title compound as off white solid (0.056 g, 0.098 mmol, 42%). ¹H NMR (400 MHz, DMSO-d6): δ 2.38 (s, 3H), 2.90 (t, 2H), 3.55-3.80 (m, 8H), 4.48 (t, 2H), 6.88 (s, 1H), 7.06 (d, 1H), 7.20-7.25 (m, 1H), 7.30-7.40 (m, 1H), 7.62-7.66 (m, 2H), 7.75-7.81 (m, 3H), 7.93-7.95 (m, 3H), 8.38 (s, 1H), 8.60 (d, 1H).

LCMS(Method-C): Retention time: 2.073 min, ES(+ve): 572.3 [M+H]⁺

### Example 2:4-(2-(2-(1-((perfluorophenyl)sulfonyl)-1H-pyrazol-4-yl)ethoxy)-6-(3-(m-tolyl)-1H-pyrazol-1-yl)pyrimidin-4-yl)morpholine

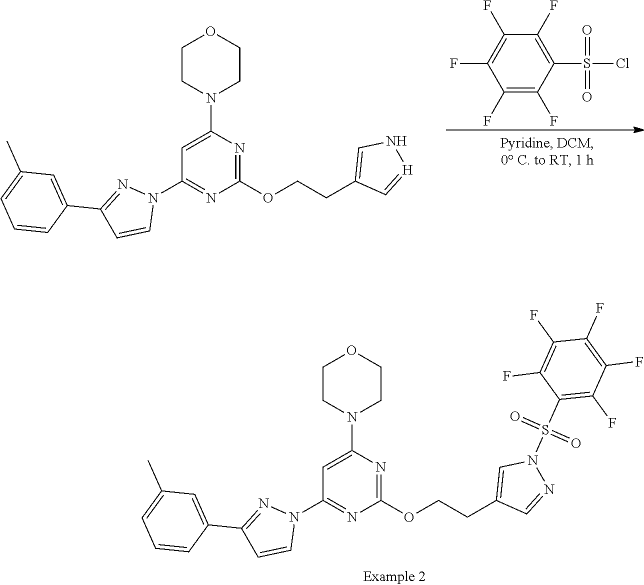

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.232 mmol) in DCM (3 mL), was added pyridine (0.1 mL, 1.159 mmol) at 0° C. under nitrogen atmosphere. After 5 min, 2,3,4,5,6-pentafluorobenzenesulfonyl chloride (0.123 g, 0.464 mmol) was added at 0° C. The reaction mixture was stirred at room temperature for 1 h. After completion of reaction, the reaction mixture was quenched with 1N HCl (10 mL) and extracted with DCM (3×30 mL). The combined organic layer was dried over anhydrous sodium sulfate, filtered and the solvent was removed in vacuum to obtain crude product. The resulting crude was purified by silica gel flash column chromatography. The desired product was eluted at 50% EtOAc in hexanes to obtain title compound as off white solid (0.056 g, 0.085 mmol, 37%).

¹H NMR (400 MHz, DMSO-d6): δ 2.39 (s, 3H), 2.97 (t, 2H), 3.63-3.77 (m, 8H), 4.53 (t, 2H), 6.90 (s, 1H), 7.06 (d, 1H), 7.22 (d, 1H), 7.35 (t, 1H), 7.78 (d, 1H), 7.82 (s, 1H), 8.08 (s, 1H), 8.50 (s, 1H), 8.60 (d, 1H).

¹⁹F NMR (400 MHz, DMSO-d6): δ−158.25_−158.39 (2F), −141.26_−141.38 (1F), −136.40-136.48 (2F).

LCMS(Method-C): Retention time: 2.181 min, ES(+ve): 662.3 [M+H]⁺

### Example 3:4-((4-(2-((4-morpholino-6-(3-(m-tolyl)-1H-pyrazol-1-yl)pyrimidin-2-yl)oxy)ethyl)-1H-pyrazol-1-yl)sulfonyl)benzonitrile

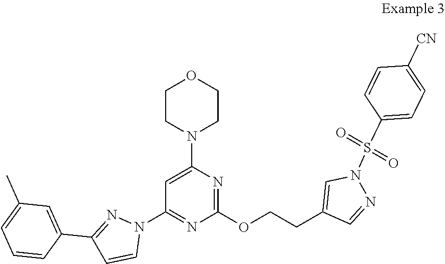 [SMILES: Cc1cccc(-c2ccn(-c3cc(N4CCOCC4)nc(OCCc4cnn(S(=O)(=O)c5ccc(C#N)cc5)c4)n3)n2)c1]

Prepared by the method of Example 2 using 4-cyanobenzenesulfonyl chloride (0.093 g, 0.464 mmol). Yield (0.066 g, 0.110 mmol, 48%).

¹H NMR (400 MHz, DMSO-d6): δ 2.33 (s, 3H), 2.91 (t, 2H), 3.60-3.68 (m, 8H), 4.49 (t, 2H), 6.88 (s, 1H), 7.06 (d, 1H), 7.21 (d, 1H), 7.35 (t, 1H), 7.78 (d, 1H), 7.82 (s, 1H), 7.96 (s, 1H), 8.11 (s, 4H), 8.44 (s, 1H), 8.59 (d, 1H).

LCMS(Method-C): Retention time: 2.053 min, ES(+ve): 597.3 [M+H]⁺

### Example 4:2,6-difluoro-4-((4-(2-((4-morpholino-6-(3-(m-tolyl)-1H-pyrazol-1-yl)pyrimidin-2-yl)oxy)ethyl)-1H-pyrazol-1-yl)sulfonyl)benzonitrile

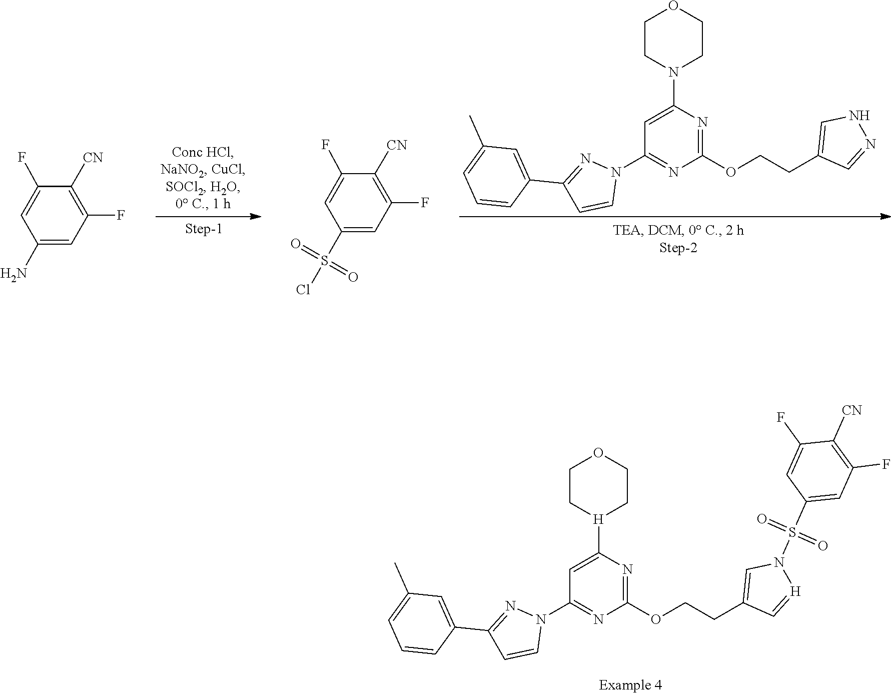

### Step 1: Synthesis of 4-cyano-3,5-difluorobenzenesulfonyl chloride

Thionyl chloride (0.48 mL, 6.481 mmol) was added to water (10 mL) at 0° C., keeping the temperature at 0° C. (very exothermic, slow and careful addition with dropping funnel). The mixture was allowed to warm to room temperature for 1 h after which CuCl (16 mg, 0.161 mmol, 0.05 eq) was added and the solution was again cooled to 0° C. (reaction mixture-1). Separately, concentrated hydrochloric acid (5 mL, 36% w/w) was added to 4-amino-2,6-difluorobenzonitrile (500 mg, 3.244 mmol) keeping the temperature below 0° C. A solution of sodium nitrite (223 mg, 3.240 mmol) in water (5 mL) was added dropwise keeping the temperature between-5 and 0° C. and the mixture stirred for an additional 15 min (reaction mixture-2). The solution of reaction mixture-2 was added dropwise to the reaction mixture-1 solution while keeping the temperature of both solutions between 0 and −5° C. and this mixture was stirred for 1 h. After completion, the reaction mixture was diluted with water (60 mL) and the aqueous layer was extracted with EtOAc (3×20 mL). The combined organic phase was dried over sodium sulfate and evaporated under reduced pressure to get crude product. The crude residue was purified by silica gel flash chromatography and pure compound was eluted at 10% EtOAc in hexanes to obtain title compound as a yellow liquid (230 mg, 0.967 mmol, 29%).

¹H NMR (400 MHz, CDCl₃): δ 7.77-7.80 (m, 2H).

¹⁹F NMR (400 MHz, CDCl₃): δ−96.22 (2F).

### Step 2: Synthesis of 2,6-difluoro-4-((4-(2-((4-morpholino-6-(3-(m-tolyl)-1H-pyrazol-1-yl)pyrimidin-2-yl)oxy)ethyl)-1H-pyrazol-1-yl)sulfonyl)benzonitrile

Prepared by the method of Example 1 using 4-cyano-3,5-difluorobenzenesulfonyl chloride (85 mg, 0.362 mmol). Yield (28 mg, 0.044 mmol, 24%).

¹H NMR (400 MHz, DMSO-d6): δ 2.39 (s, 3H), 2.95 (t, 2H), 3.63-3.76 (m, 8H), 4.51 (t, 2H), 6.89 (s, 1H), 7.07 (d, 1H), 7.21-7.24 (m, 1H), 7.34-7.37 (m, 1H), 7.77-7.79 (m, 1H), 7.81-7.83 (m, 1H), 8.04 (s, 1H), 8.10 (d, 2H), 8.45 (s, 1H), 8.59 (d, 1H).

¹⁹F NMR (400 MHz, DMSO-d6): δ−100.12 (2F).

LCMS(Method-H): Retention time: 4.060 min, ES(+ve): 633.2 [M+H]⁺

### Example 5:3,5-Difluoro-4-((4-(2-((4-morpholino-6-(3-(m-tolyl)-1H-pyrazol-1-yl)pyrimidin-2-yl)oxy)ethyl)-1H-pyrazol-1-yl)sulfonyl)benzonitrile

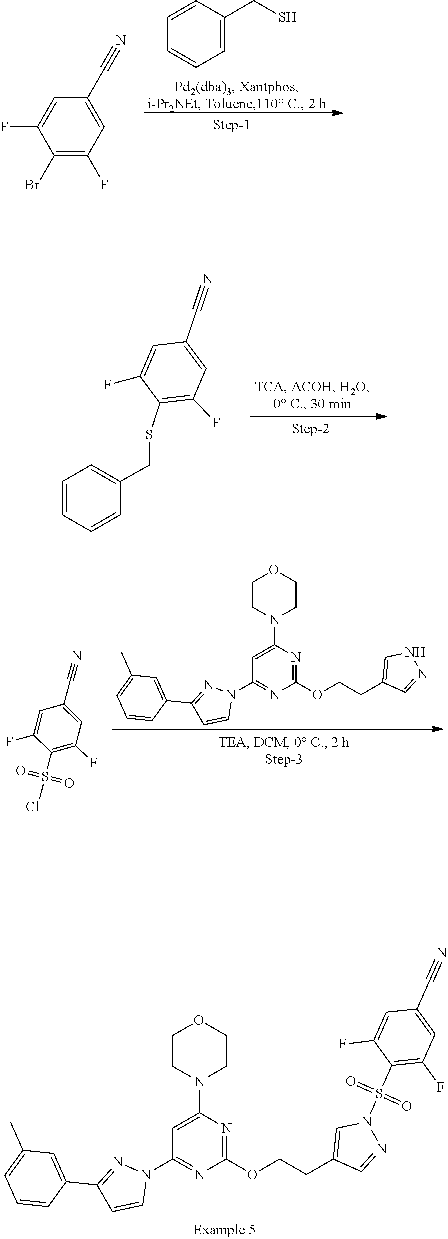 [SMILES: N#Cc1cc(F)c(Br)c(F)c1 ; SCc1ccccc1 ; N#Cc1cc(F)c(SCc2ccccc2)c(F)c1 ; N#Cc1cc(F)c(S(=O)(=O)Cl)c(F)c1 ; Cc1cccc(-c2ccn(-c3cc(N4CCOCC4)nc(OCCc4cn[nH]c4)n3)n2)c1 ; Cc1cccc(-c2ccn(-c3cc(N4CCOCC4)nc(OCCc4cnn(S(=O)(=O)c5c(F)cc(C#N)cc5F)c4)n3)n2)c1]

### Step 1: Synthesis of 4-(benzylthio)-3,5-difluorobenzonitrile

To a stirred solution of 4-bromo-3,5-difluorobenzonitrile (600 mg, 2.752 mmol) and benzyl mercaptan (0.3 mL, 2.752 mmol) in toluene (10 mL) was added DIPEA (2.43 mL, 13.762 mmol) at room temperature under nitrogen atmosphere. The reaction mixture was purged with nitrogen for 10 min. Pd₂(dba)₃ (125 mg, 0.137 mmol) and Xantphos (CAS: 161265-03-8)(156 mg, 0.275 mmol) were added, and the reaction mixture was again purged with nitrogen gas for 5 min. The reaction mixture was heated at 110° C. for 2 h. After completion, the reaction mixture was evaporated under reduced pressure to get crude product. The crude residue was purified by silica gel flash chromatography and pure compound was eluted at 10% EtOAc in hexanes to obtain title compound as off white solid (700 mg, 2.68 mmol, 97%).

¹H NMR (400 MHz, DMSO-d6): δ 4.24 (s, 2H), 7.20-7.33 (m, 5H), 7.79-7.83 (m, 2H).

¹⁹F NMR (400 MHz, DMSO-d6): δ−102.22 (2F).

### Step 2: Synthesis of 4-cyano-2,6-difluorobenzenesulfonyl chloride

To a stirred solution 4-(benzylthio)-3,5-difluorobenzonitrile (700 mg, 2.681 mmol) in acetonitrile (10 mL) at 0° C. was added a mixture of acetic acid (1 mL and water (1 mL). After 5 min, trichloroisocyanuric acid (622 mg, 2.681 mmol) was added to reaction mixture and reaction mixture was stirred at 0° C. for 30 min. After completion, the reaction mixture was quenched with 10% aq. NaHCO₃Solution (10 mL). The aqueous layer was extracted with DCM (2×30 mL). The combined DCM layers were dried over sodium sulphate and evaporated under reduced pressure to obtain crude title compound as white solid (600 mg, 2.53 mmol, 94%). The crude product was used in the next step without purification or further analysis.

### Step 3: Synthesis of 3,5-difluoro-4-((4-(2-((4-morpholino-6-(3-(m-tolyl)-1H-pyrazol-1-yl)pyrimidin-2-yl)oxy)ethyl)-1H-pyrazol-1-yl)sulfonyl)benzonitrile

Prepared by the method of Example 1 using 4-cyano-2,6-difluorobenzenesulfonyl chloride (87 mg, 0.372 mmol). Yield (17 mg, 0.027 mmol, 15%).

¹H NMR (400 MHz, DMSO-d6): δ 2.39 (s, 3H), 2.96 (t, 2H), 3.62-3.80 (m, 8H), 4.52 (t, 2H), 6.90 (s, 1H), 7.07 (d, 1H), 7.21-7.23 (m, 1H), 7.34-7.37 (m, 1H), 7.77-7.82 (m, 2H), 8.03 (s, 1H), 8.11 (d, 2H), 8.49 (s, 1H), 8.60 (d, 1H).

¹⁹F NMR (400 MHz, DMSO-d6): δ−104.00 (2F).

LCMS(Method-C): Retention time: 2.111 min, ES(+ve): 633.3 [M+H]⁺

### Example 6:4-(2-(2-(1-(4-(methylsulfonyl)phenyl)-1H-pyrazol-4-yl)ethoxy)-6-(3-(m-tolyl)-1H-pyrazol-1-yl)pyrimidin-4-yl)morpholine

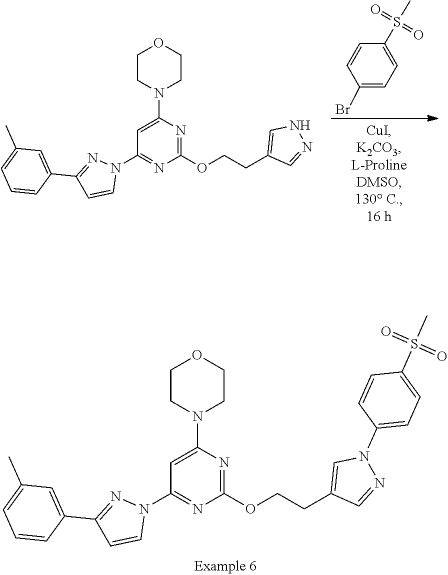 [SMILES: Cc1cccc(-c2ccn(-c3cc(N4CCOCC4)nc(OCCc4cn[nH]c4)n3)n2)c1 ; CS(=O)(=O)c1ccc(Br)cc1 ; Cc1cccc(-c2ccn(-c3cc(N4CCOCC4)nc(OCCc4cnn(-c5ccc(S(C)(=O)=O)cc5)c4)n3)n2)c1]

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.120 g, 0.278 mmol) in DMSO (2 mL), was added CuI (0.026 g, 0.139 mmol), K₂CO₃ (0.077 g, 0.417 mmol), L-Proline (0.032 g, 0.278 mmol) and 1-bromo-4-(methylsulfonyl)benzene (0.098 g, 0.583 mmol) at room temperature under nitrogen atmosphere. The reaction mixture was stirred at 130° C. for 16 h. After completion, the reaction mixture was quenched with water (20 mL) and extracted with EtOAc (3×30 mL). The combined organic layer was dried over anhydrous sodium sulfate, filtered and the solvent was removed in vacuo to obtain crude. The resulting crude was purified by silica gel flash column chromatography. The desired product was eluted at 60-80% EtOAc in hexanes to obtain title compound as off white solid (0.064 g, 0.109 mmol, 39%).

¹H NMR (400 MHz, DMSO-d6): δ 2.38 (s, 3H), 2.99 (t, 2H), 3.24 (s, 3H), 3.64-3.75 (m, 8H), 4.54 (t, 2H), 6.90 (s, 1H), 7.06 (d, 1H), 7.21 (d, 1H), 7.35 (t, 1H), 7.76-7.83 (m, 3H), 8.01-8.09 (m, 4H), 8.60-8.61 (m, 2H).

LCMS(Method-C): Retention time: 2.015 min, ES(+ve): 586.3 [M+H]⁺

### Example 7:4-(2-(2-(1-(2,3,5,6-tetrafluoro-4-(methylsulfonyl)phenyl)-1H-pyrazol-4-yl)ethoxy)-6-(3-(m-tolyl)-1H-pyrazol-1-yl)pyrimidin-4-yl)morpholine

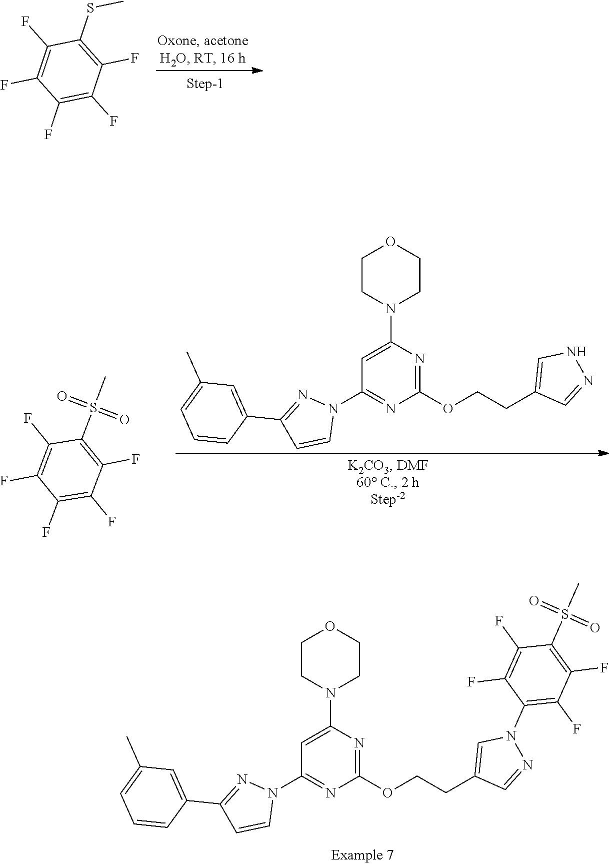 [SMILES: CSc1c(F)c(F)c(F)c(F)c1F ; CS(=O)(=O)c1c(F)c(F)c(F)c(F)c1F ; Cc1cccc(-c2ccn(-c3cc(N4CCOCC4)nc(OCCc4cn[nH]c4)n3)n2)c1 ; Cc1cccc(-c2ccn(-c3cc(N4CCOCC4)nc(OCCc4cnn(-c5c(F)c(F)c(S(C)(=O)=O)c(F)c5F)c4)n3)n2)c1]

### Step 1: Synthesis of 1,2,3,4,5-pentafluoro-6-(methylsulfonyl)benzene

In a 1 L two neck dried round bottom flask under nitrogen atmosphere, Oxone (194 g, 315 mmol) solution in water (140 mL) was introduced and cooled to 0° C. To this reaction mixture methyl (perfluorophenyl)sulfane (15 g, 70.0 mmol) in acetone (140 mL) was added dropwise at 0° C. under nitrogen atmosphere. Reaction mixture was stirred at 25° C. for 16 h. Progress of the reaction was monitored by TLC(20% EtOAc in hexane, 0.37 Rf). After the completion of reaction, reaction mixture was diluted with water (100 mL) and extracted with DCM (2×100 mL). The combined organic layers were washed water (100 mL) and brine solution (50 mL), dried over sodium sulphate and concentrated under reduced pressure to get crude product. Thus obtained crude was purified by silica gel column chromatography using 15-20% EtOAc in hexane to get pure 1,2,3,4,5-pentafluoro-6-(methylsulfonyl)benzene (14 g, 56.9 mmol, 81% yield) as white solid.

¹H NMR (400 MHz, CDCl₃): δ 3.36 (s, 3H). ¹⁹F NMR (376 MHz, CDCl₃): δ−136.02_−136.15 (2F), −143.15_−143.3 (1F), −157.47_−157.64 (2F).

### Step 2: Synthesis of 4-(2-(2-(1-(2,3,5,6-Tetrafluoro-4-(methylsulfonyl)phenyl)-1H-pyrazol-4-yl)ethoxy)-6-(3-(m-tolyl)-1H-pyrazol-1-yl)pyrimidin-4-yl)morpholine

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.231 mmol) in DMF (3 mL), was added potassium carbonate (0.064 g, 0.463 mmol) and 1,2,3,4,5-pentafluoro-6-(methylsulfonyl)benzene (0.086 g, 0.347 mmol) at room temperature under nitrogen atmosphere. The reaction mixture was stirred at 60° C. for 2 h. After completion, the reaction mixture was quenched with water (20 mL) and extracted with EtOAc (3×30 mL). The combined organic layer was dried over anhydrous sodium sulfate, filtered and the solvent was removed in vacuum to obtain crude. The resulting crude was purified by silica gel flash column chromatography. The desired product was eluted at 60-80% EtOAc in hexanes to obtain title compound as off white solid (0.036 g, 0.055 mmol, 24%).

¹H NMR (400 MHz, DMSO-d6): δ 2.38 (s, 3H), 3.01 (t, 2H), 3.56 (s, 3H), 3.65-3.77 (m, 8H), 4.54 (t, 2H), 6.90 (s, 1H), 7.06 (d, 1H), 7.21 (d, 1H), 7.35 (t, 1H), 7.77 (d, 1H), 7.82 (s, 1H), 7.96 (s, 1H), 8.15 (s, 1H), 8.60 (d, 1H).

¹⁹F NMR (400 MHz, DMSO-d6): δ−146.58_−146.66 (2F), −138.02_−138.07 (2F).

LCMS(Method-C): Retention time: 2.065 min, ES(+ve): 658.3 [M+H]⁺

### Example 8:4-(2-(2-(1-(2-bromo-3,5,6-trifluoro-4-(methylsulfonyl)phenyl)-1H-pyrazol-4-yl)ethoxy)-6-(3-(m-tolyl)-1H-pyrazol-1-yl)pyrimidin-4-yl)morpholine

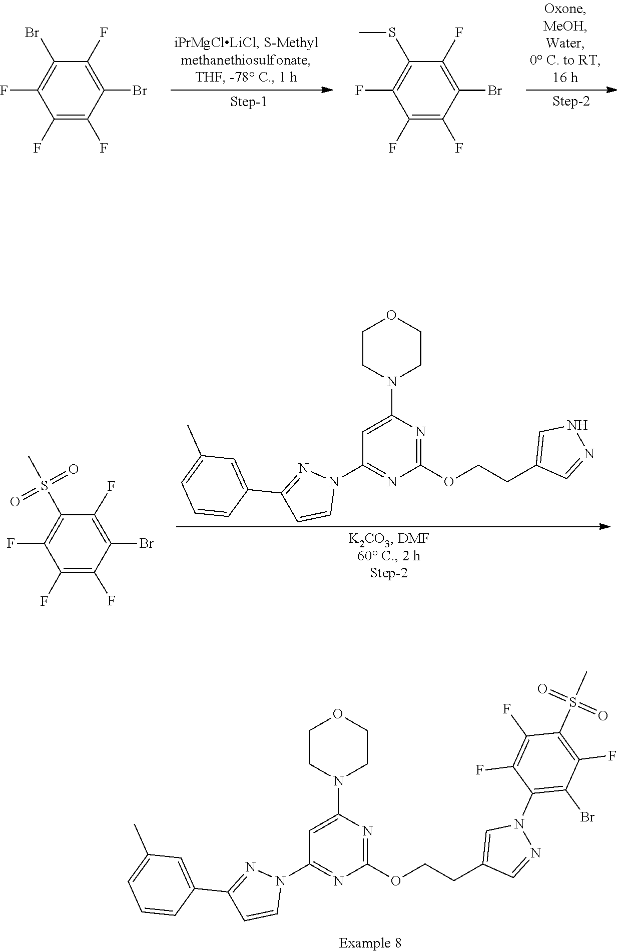 [SMILES: Fc1c(F)c(Br)c(F)c(Br)c1F ; CSc1c(F)c(F)c(F)c(Br)c1F ; CS(=O)(=O)c1c(F)c(F)c(F)c(Br)c1F ; Cc1cccc(-c2ccn(-c3cc(N4CCOCC4)nc(OCCc4cn[nH]c4)n3)n2)c1 ; Cc1cccc(-c2ccn(-c3cc(N4CCOCC4)nc(OCCc4cnn(-c5c(F)c(F)c(S(C)(=O)=O)c(F)c5Br)c4)n3)n2)c1]

### Step 1: Synthesis of (3-bromo-2,4,5,6-tetrafluorophenyl)(methyl)sulfane

To a stirred solution of 1,3-dibromo-2,4,5,6-tetrafluorobenzene (10 g, 0.0324 mol) in dry THF (100 mL) was added iPrMgCl·LiCl (1.3 M in THF)(24.9 mL, 0.0324 mol) dropwise at −78° C. under argon gas atmosphere. The reaction mixture was stirred at −78° C. for 30 min. A solution of S-Methyl methanethiosulfonate (4.09 g, 0.0324 mol) in dry THF (10 mL) was added drop wise into the reaction mixture at −78° C. The reaction mixture was stirred for 1 h. After completion, the reaction was quenched by dil. HCl (100 mL) and aqueous layer was extracted with hexanes (100 mL), dried over sodium sulphate and evaporated under reduced pressure at 30° C. to obtain crude product. The crude residue was purified by silica gel flash column chromatography. The pure compound was eluted in hexanes (100%) as colorless liquid (7 g, 0.0254 mol, 78%).

¹H NMR (400 MHz, CDCl₃): δ 2.48 (s, 3H).

### Step 2: Synthesis of 1-bromo-2,3,4,6-tetrafluoro-5-(methylsulfonyl)benzene

To a stirred solution of (3-bromo-2,4,5,6-tetrafluorophenyl)(methyl)sulfane (13 g, 0.0472 mol) in MeOH (90 mL) and water (40 mL) was added Oxone (72.63 g, 0.2363 mol) in portion wise manner at 0° C. under nitrogen atmosphere. The reaction mixture was warmed to room temperature and stirred at room temperature for 16 h. After completion, MeOH was evaporated under vacuum. The aqueous layer was extracted with DCM (2×250 mL). The combined DCM layers were dried over sodium sulphate and evaporated under reduced pressure to obtained crude product. The crude residue was purified by silica gel flash column chromatography at a gradient of 0-20% EtOAc in hexanes to obtain title compound as a white solid (10.0 g, 0.0325 mol, 69%).

¹H NMR (400 MHz, CDCl₃): δ 3.36 (s, 3H). ¹⁹F NMR (400 MHz, CDCl₃): δ−104.08_−104.12 (1F), −113.73_−113.83 (1F), −129.54_−129.64 (1F), −157.31_−157.46 (1F).

### Step 3: Synthesis of 4-(2-(2-(1-(2-bromo-3,5,6-trifluoro-4-(methylsulfonyl)phenyl)-1H-pyrazol-4-yl)ethoxy)-6-(3-(m-tolyl)-1H-pyrazol-1-yl)pyrimidin-4-yl)morpholine

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.231 mmol) in DMF (3 mL), was added K₂CO₃ (0.064 g, 0.463 mmol) and 1-bromo-2,3,4,6-tetrafluoro-5-(methylsulfonyl)benzene (0.106 g, 0.345 mmol) at room temperature under nitrogen atmosphere. The reaction mixture was stirred at 60° C. for 2 h. After completion, the reaction mixture was quenched with water (20 mL) and extracted with EtOAc (3×30 mL). The combined organic layer was dried over anhydrous sodium sulfate, filtered and the solvent was removed in vacuo to obtain crude product. The resulting crude product was purified by preparative HPLC. Fractions containing the pure compound were combined and lyophilized to obtain title compound as off white solid (0.010 g, 0.001 mmol, 6%).

Preparative HPLC Purification Method: The compound was purified on Shimadzu LC-20AP and UV detector. The column used was X-BRIDGE C18, 250 mm×19 mm, 5 μm. Column flow rate was 13.0 mL/min. Mobile phases used were: (A) 0.1% NH₃ in water and (B) 100% MeCN. The gradient expressed as % solvent (B) was 40% over 0.01 min, 70% over 22 min, 70% over 35 min, 100% over 35.01 min, 100% over 37 min, 40% over 37.01 min, and 40% over 43 min. The UV spectra were recorded at 220 nm and 254 nm Lambdamax.

¹H NMR (400 MHz, DMSO-d6): δ 2.38 (s, 3H), 3.01 (t, 2H), 3.52 (s, 3H), 3.65-3.75 (m, 8H), 4.54 (t, 2H), 6.90 (s, 1H), 7.04 (d, 1H), 7.23 (d, 1H), 7.35 (t, 1H), 7.76 (d, 1H), 7.80 (s, 1H), 7.89 (s, 1H), 8.02 (s, 1H), 8.60 (d, 1H). ¹⁹F NMR (400 MHz, DMSO-d6): δ−143.84_−143.94 (1F), −131.91_−131.97 (1F), −103.33_-103.36 (1F).

LCMS(Method-C): Retention time: 2.129 min, ES(+ve): 718.3/720.3 [M+H]⁺

### Example 9:4-(2-(2-(1-(3-(methylsulfonyl)phenyl)-1H-pyrazol-4-yl)ethoxy)-6-(3-(m-tolyl)-1H-pyrazol-1-yl)pyrimidin-4-yl)morpholine

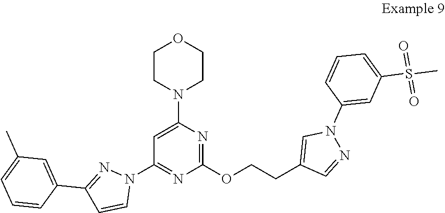 [SMILES: Cc1cccc(-c2ccn(-c3cc(N4CCOCC4)nc(OCCc4cnn(-c5cccc(S(C)(=O)=O)c5)c4)n3)n2)c1]

Prepared by the method of Example 6 using 1-bromo-3-(methylsulfonyl)benzene (0.137 g, 0.583 mmol). Yield (0.034 g, 0.058 mmol, 21%).

¹H NMR (400 MHz, DMSO-d6): δ 2.38 (s, 3H), 2.99 (t, 2H), 3.25 (s, 3H), 3.62-3.75 (m, 8H), 4.54 (t, 2H), 6.90 (s, 1H), 7.06 (d, 1H), 7.21 (d, 1H), 7.35 (t, 1H), 7.74-7.82 (m, 5H), 8.15-8.17 (m, 1H), 8.32 (s, 1H), 8.58-8.64 (m, 2H).

LCMS(Method-C): Retention time: 2.008 min, ES(+ve): 586.3 [M+H]⁺

### Example 10:4-(2-(2-(1-(2,3,4,6-tetrafluoro-5-(methylsulfonyl)phenyl)-1H-pyrazol-4-yl)ethoxy)-6-(3-(m-tolyl)-1H-pyrazol-1-yl)pyrimidin-4-yl)morpholine

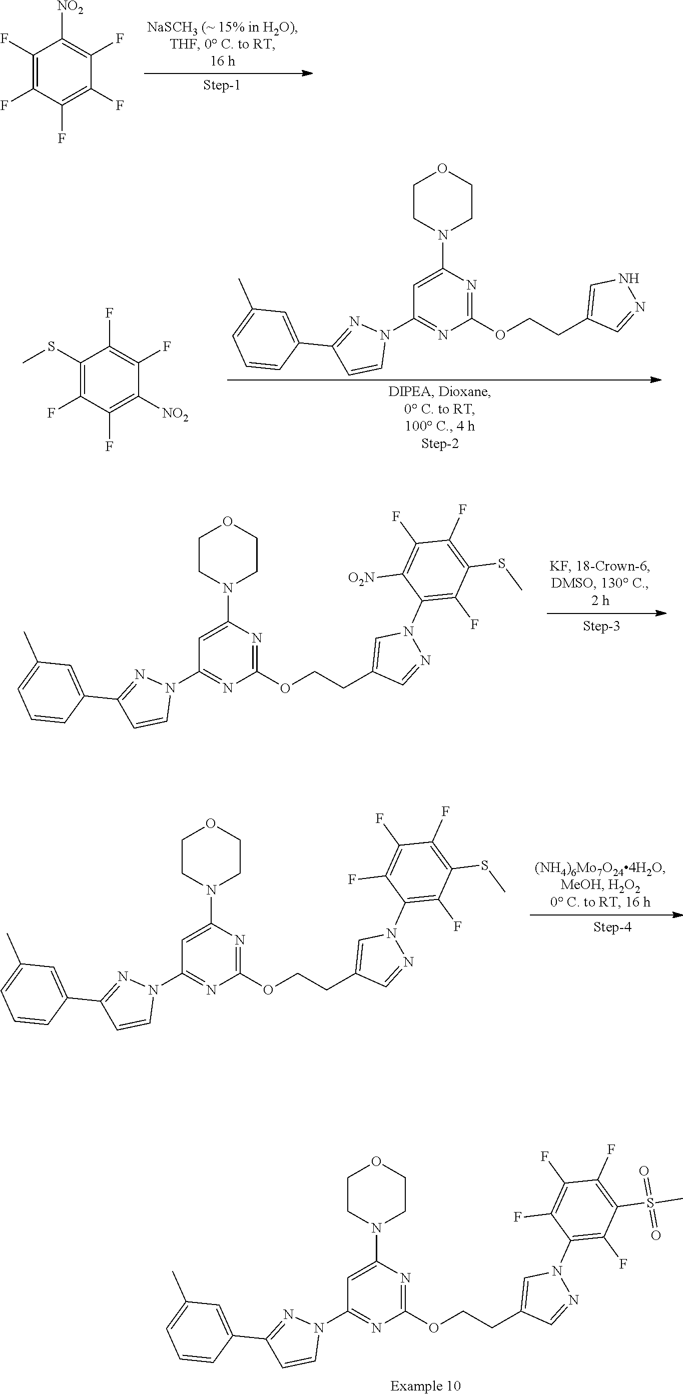 [SMILES: O=[N+]([O-])c1c(F)c(F)c(F)c(F)c1F ; CSc1c(F)c(F)c([N+](=O)[O-])c(F)c1F ; Cc1cccc(-c2ccn(-c3cc(N4CCOCC4)nc(OCCc4cn[nH]c4)n3)n2)c1 ; CSc1c(F)c(F)c([N+](=O)[O-])c(-n2cc(CCOc3nc(N4CCOCC4)cc(-n4ccc(-c5cccc(C)c5)n4)n3)cn2)c1F ; CSc1c(F)c(F)c(F)c(-n2cc(CCOc3nc(N4CCOCC4)cc(-n4ccc(-c5cccc(C)c5)n4)n3)cn2)c1F ; Cc1cccc(-c2ccn(-c3cc(N4CCOCC4)nc(OCCc4cnn(-c5c(F)c(F)c(F)c(S(C)(=O)=O)c5F)c4)n3)n2)c1]

### Step 1: Synthesis of methyl (2,3,5,6-tetrafluoro-4-nitrophenyl)sulfane

To a stirred solution of 1,2,3,4,5-pentafluoro-6-nitrobenzene (5.00 g, 0.023 mmol) in tetrahydrofuran (50 mL), was added sodium thiomethoxide as a ~15% in H₂O (10 mL, 0.023 mmol) at 0° C. The reaction mixture was warmed to room temperature and stirred for 16 h. The reaction mixture was quenched with cold water (100 mL) and the aqueous layer was extracted with DCM (3×100 mL). Combined organic layer was dried over anhydrous sodium sulfate, filtered and concentrated under reduced pressure to give the crude product. The crude residue was purified by silica gel flash chromatography. The desired product was eluted with 40-60% EtOAc in hexane to obtain title compound as brown liquid (1.500 g, 0.006 mmol, 26%).

¹H NMR (400 MHz, DMSO-d6): δ 2.66 (m, 3H).

¹⁹F NMR (400 MHz, DMSO-d6): δ−146.86_−146.91 (2F), −134.00_−134.04 (2F).

### Step 2: Synthesis of 4-(6-(3-(m-tolyl)-1H-pyrazol-1-yl)-2-(2-(1-(2,4,5-trifluoro-3-(methylthio)-6-nitrophenyl)-1H-pyrazol-4-yl)ethoxy)pyrimidin-4-yl)morpholine

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.250 g, 0.580 mmol) and methyl (2,3,5,6-tetrafluoro-4-nitrophenyl)sulfane (0.279 g, 1.157 mmol) in 1,4-dioxane (3 mL) was added DIPEA (0.3 mL, 0.580 mmol). The reaction mixture was heated at 100° C. for 4 h. After completion, the reaction mixture was evaporated under reduced pressure to give crude product.

The crude residue was purified by silica gel flash chromatography. The desired product was eluted with 20-40% EtOAc in hexanes to obtain title compound as brown solid (0.240 g, 0.368 mmol, 35%).

LCMS(Method-C): Retention time: 2.357 min, ES(+ve): 653.3 [M+H]⁺

### Step 3: Synthesis of 4-(2-(2-(1-(2,3,4,6-tetrafluoro-5-(methylthio)phenyl)-1H-pyrazol-4-yl)ethoxy)-6-(3-(m-tolyl)-1H-pyrazol-1-yl)pyrimidin-4-yl)morpholine

To a stirred solution of 4-(6-(3-(m-tolyl)-1H-pyrazol-1-yl)-2-(2-(1-(2,4,5-trifluoro-3-(methylthio)-6-nitrophenyl)-1H-pyrazol-4-yl)ethoxy)pyrimidin-4-yl)morpholine (0.150 g, 0.230 mmol) in DMSO (2.5 mL) was added 18-Crown-6 (0.03 g, 0.113 mmol) and KF (0.040 g, 0.689 mmol) at room temperature. The resulting reaction mixture was stirred at 130° C. for 2 h. The reaction mixture was quenched with cold water (20 mL) and aqueous layer was extracted with EtOAc (3×20 mL). Combined organic layer was dried over anhydrous sodium sulfate, filtered and concentrated under reduced pressure to give the crude product. The crude product was purified by silica gel flash chromatography. The desired product was eluted with 30-50% EtOAc in hexanes to obtain title compound as yellow solid (0.097 g, 0.155 mmol, 67%).

¹H NMR (400 MHz, DMSO-d6): δ 2.38 (s, 3H), 2.49 (s, 3H), 2.99 (t, 2H), 3.65-3.75 (m, 8H), 4.52 (t, 2H), 6.90 (s, 1H), 7.05 (d, 1H), 7.21 (d, 1H), 7.34 (t, 1H), 7.75-7.83 (m, 3H), 8.03 (s, 1H), 8.61 (d, 1H).

LCMS(Method-C): Retention time: 2.378 min, ES(+ve): 626.3 [M+H]⁺

### Step 4: Synthesis of 4-(2-(2-(1-(2,3,4,6-Tetrafluoro-5-(methylsulfonyl)phenyl)-1H-pyrazol-4-yl)ethoxy)-6-(3-(m-tolyl)-1H-pyrazol-1-yl)pyrimidin-4-yl)morpholine

To a stirred solution of 4-(2-(2-(1-(2,3,4,6-tetrafluoro-5-(methylthio)phenyl)-1H-pyrazol-4-yl)ethoxy)-6-(3-(m-tolyl)-1H-pyrazol-1-yl)pyrimidin-4-yl)morpholine (0.087 g, 0.139 mmol) in MeOH (5.0 mL) was added ammonium molybdate tetrahydrate (0.100 g, 0.080 mmol) and 30% w/v aqueous H₂O₂ (1.0 mL) at 0° C. under nitrogen atmosphere. The reaction mixture was stirred at room temperature for 16 h. The reaction mixture was quenched with water (20 mL) and aqueous layer was extracted with DCM (3×20 mL). Combined organic layer was dried over anhydrous sodium sulfate, filtered and concentrated under reduced pressure to give the crude product. The crude residue was purified by silica gel flash chromatography. The desired product was eluted with 30-50% EtOAc in hexanes to obtain title compound as brown solid (0.010 g, 0.132 mmol, 11%).

^{1\}H NMR (400 MHz, DMSO-d6): δ 2.39 (s, 3H), 3.01 (t, 2H), 3.54 (s, 3H), 3.65-3.77 (m, 8H), 4.54 (t, 2H), 6.92 (s, 1H), 7.06 (d, 1H), 7.22 (d, 1H), 7.36 (t, 1H), 7.78 (d, 1H), 7.82 (s, 1H), 7.89 (s, 1H), 8.07 (s, 1H), 8.61 (d, 1H).

¹⁹F NMR (400 MHz, DMSO-d6): δ−160.23_−160.38 (1F), −132.09_−132.18 (1F), −130.56-130.65 (1F), −121.87_−121.89 (1F).

LCMS(Method-C): Retention time: 1.893 min, ES(+ve): 658.3 [M+H]⁺

### Example 11:4-(2-(2-(1-(pyridin-4-yl)-1H-pyrazol-4-yl)ethoxy)-6-(3-(m-tolyl)-1H-pyrazol-1-yl)pyrimidin-4-yl)morpholine

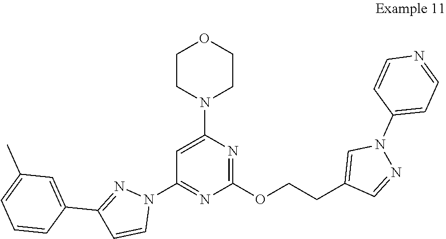 [SMILES: Cc1cccc(-c2ccn(-c3cc(N4CCOCC4)nc(OCCc4cnn(-c5ccncc5)c4)n3)n2)c1]

Prepared by a method generally analogous to Example 6 using 4-iodopyridine (71 mg, 0.348 mmol). The crude product was purified using reversed phase column chromatography eluting with 70% acetonitrile in water. Fractions containing pure compound were combined and lyophilized to obtain the title compound. Yield (43 mg, 0.084 mmol, 36%).

¹H NMR (400 MHz, DMSO-d6): δ 2.37 (s, 3H), 2.98 (t, 2H), 3.62-3.77 (m, 8H), 4.53 (t, 2H), 6.89 (s, 1H), 7.06 (t, 1H), 7.20 (d, 1H), 7.34 (t, 1H), 7.75-7.88 (m, 5H), 8.59-8.63 (m, 4H).

LCMS(Method-C): Retention time: 1.762 min, ES(+ve): 509.3 [M+H]⁺

### Example 12:4-(2-(2-(1-(2-fluoropyridin-4-yl)-1H-pyrazol-4-yl)ethoxy)-6-(3-(m-tolyl)-1H-pyrazol-1-yl)pyrimidin-4-yl)morpholine

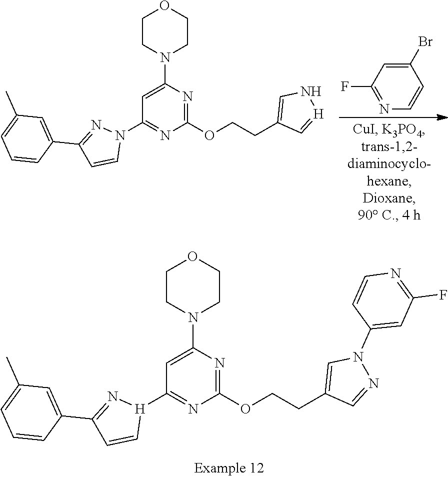

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.232 mmol) in dioxane (5 mL), was added CuI (0.022 g, 0.116 mmol), K₃PO₄ (0.098 g, 0.462 mmol), trans-1,2-diaminocyclohexane (0.099 g, 0.462 mmol) and 4-bromo-2-fluoropyridine (0.122 g, 0.693 mmol) at room temperature under nitrogen atmosphere. The reaction mixture was stirred at 90° C. for 4 h. After completion, the reaction mixture was quenched with water (20 mL) and extracted with EtOAc (3×30 mL). The combined organic layer was dried over anhydrous sodium sulfate, filtered and the solvent was removed in vacuo to obtain crude. The resulting crude was purified by RP-flash chromatography. Product was eluted at 70% acetonitrile in water. Fractions containing pure compound were combined and lyophilized to obtain title compound as off white solid (0.037 g, 0.070 mmol, 30%).

¹H NMR (400 MHz, DMSO-d6): δ 2.38 (s, 3H), 2.99 (t, 2H), 3.62-3.75 (m, 8H), 4.54 (t, 2H), 6.90 (s, 1H), 7.05-7.07 (m, 1H), 7.20 (d, 1H), 7.34 (t, 1H), 7.58 (d, 1H), 7.76-7.81 (m, 3H), 7.89 (s, 1H), 8.28-8.29 (m, 1H), 8.59-8.60 (m, 1H), 8.68 (s, 1H).

¹⁹F NMR (400 MHz, DMSO-d6): δ−67.01 (1F).

LCMS(Method-C): Retention time: 2.164 min, ES(+ve): 527.3 [M+H]⁺

### Example 13:4-(2-(2-(1-(perfluoropyridin-4-yl)-1H-pyrazol-4-yl)ethoxy)-6-(3-(m-tolyl)-1H-pyrazol-1-yl)pyrimidin-4-yl)morpholine

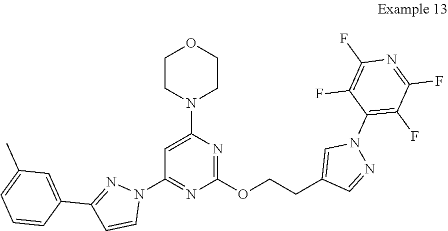 [SMILES: Cc1cccc(-c2ccn(-c3cc(N4CCOCC4)nc(OCCc4cnn(-c5c(F)c(F)nc(F)c5F)c4)n3)n2)c1]

Prepared by the method of Example 7, Step 2 using perfluoropyridine (70 mg, 0.417 mmol). After completion, the reaction mixture was quenched with ice cold water. The aqueous layer was extracted with EtOAc (3×30 mL). The combined EtOAc layer was dried over sodium sulfate and evaporated under reduced pressure to obtain crude product. The crude residue was purified by RP-flash chromatography by eluting with acetonitrile in water. After lyophilization title compound was obtained as a white solid (90 mg, 0.155 mmol, 55%).

¹H NMR (400 MHz, DMSO-d6): δ 2.39 (s, 3H), 3.03 (t, 2H), 3.65-3.75 (m, 8H), 4.55 (t, 2H), 6.91 (s, 1H), 7.06 (d, 1H), 7.22 (d, 1H), 7.35 (t, 1H), 7.78 (d, 1H), 7.82 (s, 1H), 8.03 (s, 1H), 8.23 (s, 1H), 8.60 (d, 1H).

¹⁹F NMR (400 MHz, DMSO-d6): δ−90.72_−90.81 (2F), −149.41_−149.51 (2F).

LCMS(Method-C): Retention time: 2.205 min, ES(+ve): 581.3 [M+H]⁺

### Example 14:4-(2-(2-(1-(6-fluoropyridin-3-yl)-1H-pyrazol-4-yl)ethoxy)-6-(3-(m-tolyl)-1H-pyrazol-1-yl)pyrimidin-4-yl)morpholine

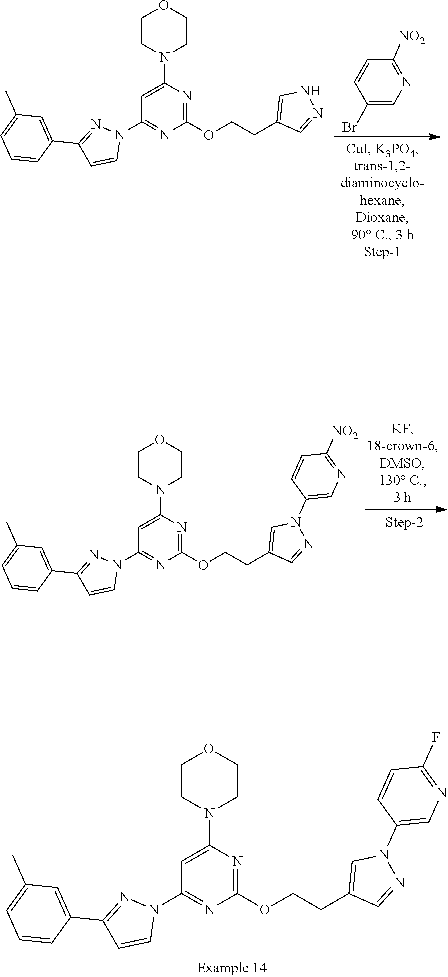 [SMILES: Cc1cccc(-c2ccn(-c3cc(N4CCOCC4)nc(OCCc4cn[nH]c4)n3)n2)c1 ; O=[N+]([O-])c1ccc(Br)cn1 ; Cc1cccc(-c2ccn(-c3cc(N4CCOCC4)nc(OCCc4cnn(-c5ccc([N+](=O)[O-])nc5)c4)n3)n2)c1 ; Cc1cccc(-c2ccn(-c3cc(N4CCOCC4)nc(OCCc4cnn(-c5ccc(F)nc5)c4)n3)n2)c1]

### Step 1: Synthesis of 4-(2-(2-(1-(6-nitropyridin-3-yl)-1H-pyrazol-4-yl)ethoxy)-6-(3-(m-tolyl)-1H-pyrazol-1-yl)pyrimidin-4-yl)morpholine

Prepared by a method generally analogous to Example 12 using 5-bromo-2-nitropyridine (0.282 g, 1.389 mmol). After completion, the reaction mixture was quenched with water (20 mL) and extracted with DCM (3×30 mL). The combined organic layer was dried over anhydrous sodium sulfate, filtered and the solvent was removed in vacuo to obtain crude. The crude residue was purified by silica gel flash chromatography. The desired product was eluted with 60-65% EtOAc in hexanes to obtain title compound as yellow solid (0.060 g, 0.108 mmol, 47%).

LCMS(Method-C): Retention time: 2.109 min, ES(+ve): 554.3 [M+H]⁺.

### Step 2: Synthesis of 4-(2-(2-(1-(6-fluoropyridin-3-yl)-1H-pyrazol-4-yl)ethoxy)-6-(3-(m-tolyl)-1H-pyrazol-1-yl)pyrimidin-4-yl)morpholine

Prepared by a method generally analogous to Example 10, Step 3, using 4-(2-(2-(1-(6-nitropyridin-3-yl)-1H-pyrazol-4-yl)ethoxy)-6-(3-(m-tolyl)-1H-pyrazol-1-yl)pyrimidin-4-yl)morpholine (0.060 g, 0.108 mmol). The crude product was purified by RP-flash chromatography. Product was eluted at 45% acetonitrile in water. Fractions containing pure compound were combined and lyophilized to obtain title compound as off white solid (0.019 g, 0.070 mmol, 30%).

¹H NMR (400 MHz, DMSO-d6): δ 2.39 (s, 3H), 2.99 (t, 2H), 3.65-3.75 (m, 8H), 4.54 (t, 2H), 6.91 (s, 1H), 7.06 (d, 1H), 7.22 (d, 1H), 7.34-7.37 (m, 2H), 7.77-7.82 (m, 3H), 8.38-842 (m, 1H), 8.51 (s, 1H), 8.61 (d, 1H), 8.70-8.71 (m, 1H). ¹⁹F NMR (400 MHz, DMSO-d6): δ−72.39 (1F).

LCMS(Method-J): Retention time: 4.194 min, ES(+ve): 527.2 [M+H]⁺

### Example 15:4-(2-(2-(1-(2-fluoropyrimidin-4-yl)-1H-pyrazol-4-yl)ethoxy)-6-(3-(m-tolyl)-1H-pyrazol-1-yl)pyrimidin-4-yl)morpholine

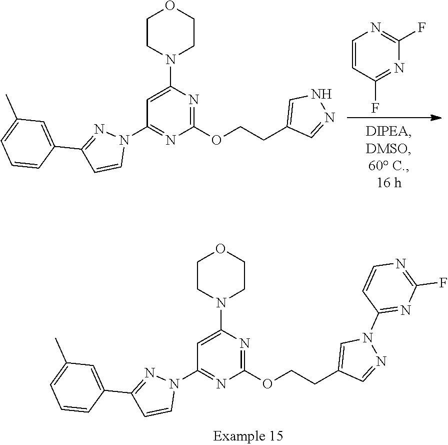 [SMILES: Cc1cccc(-c2ccn(-c3cc(N4CCOCC4)nc(OCCc4cn[nH]c4)n3)n2)c1 ; Fc1ccnc(F)n1 ; Cc1cccc(-c2ccn(-c3cc(N4CCOCC4)nc(OCCc4cnn(-c5ccnc(F)n5)c4)n3)n2)c1]

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.231 mmol) in DMSO (2 mL), was added DIPEA (0.2 mL, 1.390 mmol) and 2,4-difluoropyrimidine (0.100 g, 0.926 mmol) at room temperature under nitrogen atmosphere. The reaction mixture was stirred at 60° C. for 16 h. After completion, the reaction mixture was quenched with cold water (20 mL) and extracted with EtOAc (3×20 mL). The combined organic layer was dried over anhydrous sodium sulfate, filtered and the solvent was removed in vacuum to obtain crude product. The resulting crude product was purified by RP-flash column chromatography. The product was eluted at 70% acetonitrile in water. Fractions containing pure compound were combined and lyophilized to obtain title compound as white solid (0.025 g, 0.047 mmol, 20%).

¹H NMR (400 MHz, DMSO-d6): δ 2.38 (s, 3H), 3.00 (t, 2H), 3.63-3.76 (m, 8H), 4.55 (t, 2H), 6.89 (s, 1H), 7.05 (d, 1H), 7.20 (d, 1H), 7.34 (t, 1H), 7.76 (d, 1H), 7.81 (s, 1H), 7.83-7.86 (m, 1H), 7.99 (s, 1H), 8.60 (d, 2H), 8.78-8.79 (m, 1H).

¹⁹F NMR (400 MHz, DMSO-d6): δ−45.62 (1F).

LCMS(Method-C): Retention time: 2.091 min, ES(+ve): 528.4 [M+H]⁺

### Example 16:4-(2-(2-(1-(2,5-difluoropyrimidin-4-yl)-1H-pyrazol-4-yl)ethoxy)-6-(3-(m-tolyl)-1H-pyrazol-1-yl)pyrimidin-4-yl)morpholine

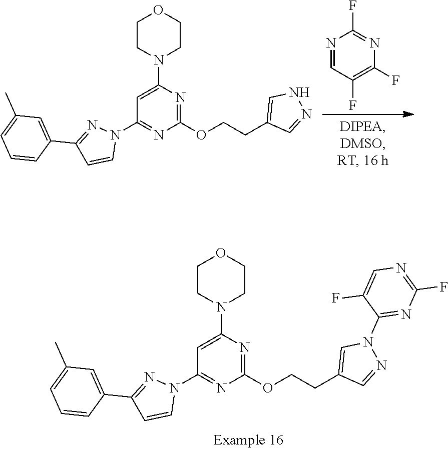 [SMILES: Cc1cccc(-c2ccn(-c3cc(N4CCOCC4)nc(OCCc4cn[nH]c4)n3)n2)c1 ; Fc1ncc(F)c(F)n1 ; Cc1cccc(-c2ccn(-c3cc(N4CCOCC4)nc(OCCc4cnn(-c5nc(F)ncc5F)c4)n3)n2)c1]

To a stirred solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(0.100 g, 0.231 mmol) in DMSO (3 mL), was added DIPEA (0.12 mL, 0.696 mmol) and 2,4,5-trifluoropyrimidine (0.062 g, 0.462 mmol) at room temperature under nitrogen atmosphere. The reaction mixture was stirred at room temperature for 16 h. After completion, the reaction mixture was quenched with water (20 mL) and extracted with EtOAc (3×20 mL). The combined organic layer was dried over anhydrous sodium sulfate, filtered and the solvent was removed in vacuum to obtain crude product. The crude residue was purified by silica gel flash chromatography. The desired product was eluted with 70% EtOAc in hexanes to obtain title compound as an off-white solid (0.035 g, 0.064 mmol, 27%).

¹H NMR (400 MHz, DMSO-d6): δ 2.38 (s, 3H), 3.02 (t, 2H), 3.64-3.76 (m, 8H), 4.55 (t, 2H), 6.90 (s, 1H), 7.05 (d, 1H), 7.21 (d, 1H), 7.35 (t, 1H), 7.77 (d, 1H), 7.81 (s, 1H), 8.04 (s, 1H), 8.58-8.60 (m, 2H), 8.95 (d, 1H).

¹⁹F NMR (400 MHz, DMSO-d6): δ−145.67_−145.74 (1F), −50.92_−51.00 (1F).

LCMS(Method-C): Retention time: 2.094 min, ES(+ve): 546.5 [M+H]⁺

### Example 17:4-(2-(2-(1-(6-fluoropyrimidin-4-yl)-1H-pyrazol-4-yl)ethoxy)-6-(3-(m-tolyl)-1H-pyrazol-1-yl)pyrimidin-4-yl)morpholine

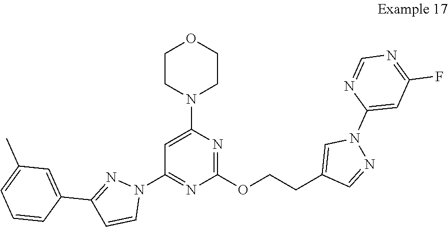 [SMILES: Cc1cccc(-c2ccn(-c3cc(N4CCOCC4)nc(OCCc4cnn(-c5cc(F)ncn5)c4)n3)n2)c1]

Prepared by the method of Example 16 using 4,6-difluoropyrimidine (0.065 g, 0.560 mmol). The crude product was purified by RP-flash column chromatography. The product was eluted at 80% acetonitrile in water. Fractions containing pure compound were combined and lyophilized to obtain title compound as white solid (0.015 g, 0.028 mmol, 10%).

¹H NMR (400 MHz, DMSO-d6): δ 2.38 (s, 3H), 3.00 (t, 2H), 3.63-3.75 (m, 8H), 4.55 (t, 2H), 6.89 (s, 1H), 7.05 (d, 1H), 7.20 (d, 1H), 7.34 (t, 1H), 7.59 (s, 1H), 7.76 (d, 1H), 7.81 (s, 1H), 7.99 (s, 1H), 8.60 (d, 1H), 8.63 (s, 1H), 8.87 (s, 1H). ¹⁹F NMR (400 MHz, DMSO-d6): δ−59.53 (1F).

LCMS(Method-J): Retention time: 4.026 min, ES(+ve): 528.4 [M+H]⁺

### Example 18:4-(2-(2-(1-(4-fluoropyrimidin-2-yl)-1H-pyrazol-4-yl)ethoxy)-6-(3-(m-tolyl)-1H-pyrazol-1-yl)pyrimidin-4-yl)morpholine

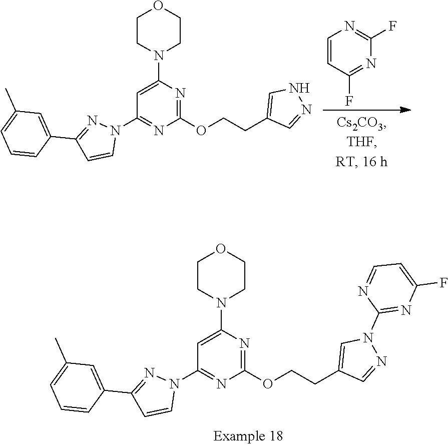 [SMILES: Cc1cccc(-c2ccn(-c3cc(N4CCOCC4)nc(OCCc4cn[nH]c4)n3)n2)c1 ; Fc1ccnc(F)n1 ; Cc1cccc(-c2ccn(-c3cc(N4CCOCC4)nc(OCCc4cnn(-c5nccc(F)n5)c4)n3)n2)c1]

A solution of 4-(2-(2-(1H-pyrazol-4-yl)ethoxy)-6-(3-(m-tolyl)-1H-pyrazol-1-yl)pyrimidin-4-yl)morpholine (WO2021163727 A1)(1.0 g, 2.317 mmol) in THF (15 mL) was stirred for 5 min at room temperature. To the resulting solution, Cs₂CO₃ (2.259 g, 6.952 mmol) and 2,4-difluoropyrimidine (1.075 g, 9.269 mmol) were added at room temperature under nitrogen atmosphere. The reaction mixture was stirred at room temperature for 16 h. After completion, the reaction mixture was quenched with cold water (100 mL) and extracted with EtOAc (3×100 mL). The combined organic layer was dried over anhydrous sodium sulfate, filtered and the solvent was removed in vacuo to obtain crude. The resulting crude was purified by RP-Flash column chromatography. Product was eluted at 45-65% MeCN in water. Fractions containing pure compound were combined and lyophilized to obtain title compound as a white solid (0.061 g, 0.115 mmol, 5%).

¹H NMR (400 MHz, DMSO-d6): δ 2.38 (s, 3H), 3.00 (t, 2H), 3.69 (s, 8H), 4.54 (t, 2H), 6.89 (s, 1H), 7.05 (d, 1H), 7.21 (d, 1H), 7.30-7.36 (m, 2H), 7.77 (d, 1H), 7.81 (s, 1H), 7.89 (s, 1H), 8.58-8.61 (m, 2H), 8.92-8.96 (m, 1H).

¹⁹F NMR (400 MHz, DMSO-d6): δ−58.596 (1F).

LCMS(Method-C): Retention time: 2.011 min, ES(+ve): 528.4 [M+H]⁺

### Example 19: N-(2-(1-(4-fluoropyridin-2-yl)-1H-pyrazol-4-yl)ethyl)-N-methyl-4-morpholino-6-(3-phenyl-1H-pyrazol-1-yl)pyrimidin-2-amine

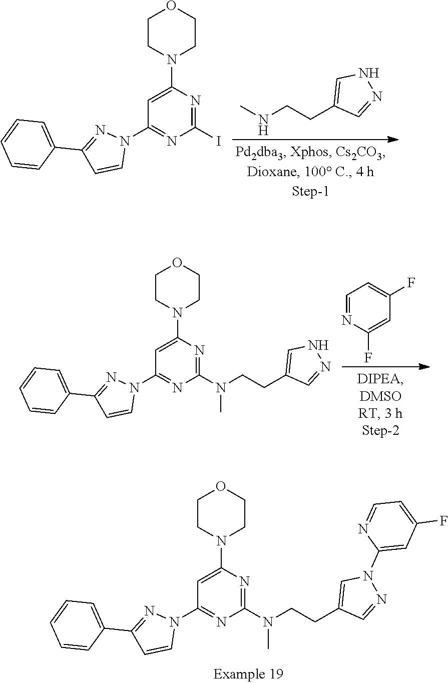

### Step 1: Synthesis of N-(2-(1H-pyrazol-4-yl)ethyl)-N-methyl-4-morpholino-6-(3-phenyl-1H-pyrazol-1-yl)pyrimidin-2-amine

To a stirred solution of 4-(2-iodo-6-(3-phenyl-1H-pyrazol-1-yl)pyrimidin-4-yl)morpholine (WO2021163727 A1)(2.0 g, 4.616 mmol) and N-methyl-2-(1H-pyrazol-4-yl) ethan-1-amine (0.86 g, 6.927 mmol) in dioxane (20 mL) was added Cs₂CO₃ (6.0 g, 18.472 mmol) at room temperature under nitrogen atmosphere. The reaction mixture was purged with nitrogen gas for 10 min. Pd₂(dba)₃ (0.634 g, 0.692 mmol) and XPhos (CAS: 564483-18-7)(1.1 g, 2.309 mmol) were added and the reaction mixture was heated at 100° C. for 4 h. After completion, the reaction mixture was diluted with water (50 mL) and the aqueous layer was extracted with EtOAc (3×100 mL). The combined organic phases were dried over sodium sulfate and evaporated under reduced pressure. The crude residue was purified by silica gel flash chromatography and pure compound was eluted at 30% EtOAc in hexanes to obtain title compound as a brown solid (1.40 g, 3.251 mmol, 70%).

¹H NMR (400 MHz, DMSO-d6): δ 2.73 (t, 2H), 3.11 (s, 3H), 3.57-3.75 (m, 10H), 6.56 (s, 1H), 7.04 (d, 1H), 7.37-7.40 (m, 2H), 7.44-7.48 (m, 3H), 7.96-7.98 (m, 2H), 8.60 (s, 1H), 12.54 (s, 1H).

### Step 2: Synthesis of N-(2-(1-(4-fluoropyridin-2-yl)-1H-pyrazol-4-yl)ethyl)-N-methyl-4-morpholino-6-(3-phenyl-1H-pyrazol-1-yl)pyrimidin-2-amine

To a stirred solution of N-(2-(1H-pyrazol-4-yl)ethyl)-N-methyl-4-morpholino-6-(3-phenyl-1H-pyrazol-1-yl)pyrimidin-2-amine (0.150 g, 0.348 mmol) in DMSO (1 mL), was added DIPEA (0.24 mL, 1.394 mmol) and 2,4-difluoropyridine (0.080 g, 0.697 mmol) at room temperature under nitrogen atmosphere. The reaction mixture was stirred at room temperature for 3 h. After completion, the reaction mixture was quenched by water (40 mL) and extracted with EtOAc (2×50 mL). The combined organic layer was dried over anhydrous sodium sulfate, filtered and the solvent was removed in vacuum to obtain crude product. The resulting crude product was purified by column chromatography. The product was eluted at 25% EtOAc in hexanes to obtain title compound as white solid (0.080 g, 0.152 mmol, 43%).

¹H NMR (400 MHz, DMSO-d6): δ 2.85 (t, 2H), 3.15 (s, 3H), 3.55-3.75 (m, 8H), 3.83-3.95 (m, 2H), 6.55 (s, 1H), 7.03 (d, 1H), 7.24-7.28 (m, 1H), 7.37-7.41 (m, 1H), 7.45-7.49 (m, 2H), 7.62-7.65 (m, 1H), 7.77 (s, 1H), 7.96-7.98 (m, 2H), 8.46-8.51 (m, 2H), 8.63 (d, 1H). ¹⁹F NMR (400 MHz, DMSO-d6): δ−99.75 (1F).

LCMS(Method-H): Retention time: 4.743 min, ES(+ve): 526.4 [M+H]⁺

### Example 20: N-(2-(1-(2-fluoropyrimidin-4-yl)-1H-pyrazol-4-yl)ethyl)-N-methyl-4-morpholino-6-(3-phenyl-1H-pyrazol-1-yl)pyrimidin-2-amine and Example 21: N-(2-(1-(4-fluoropyrimidin-2-yl)-1H-pyrazol-4-yl)ethyl)-N-methyl-4-morpholino-6-(3-phenyl-1H-pyrazol-1-yl)pyrimidin-2-amine

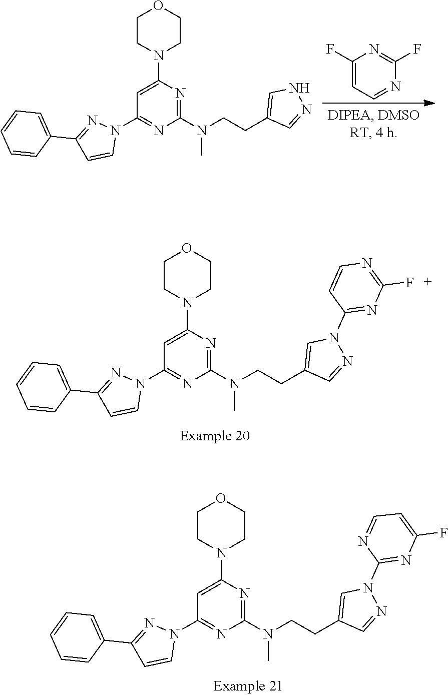 [SMILES: CN(CCc1cn[nH]c1)c1nc(N2CCOCC2)cc(-n2ccc(-c3ccccc3)n2)n1 ; Fc1ccnc(F)n1 ; CN(CCc1cnn(-c2ccnc(F)n2)c1)c1nc(N2CCOCC2)cc(-n2ccc(-c3ccccc3)n2)n1 ; CN(CCc1cnn(-c2nccc(F)n2)c1)c1nc(N2CCOCC2)cc(-n2ccc(-c3ccccc3)n2)n1]

To a stirred solution of N-(2-(1H-pyrazol-4-yl)ethyl)-N-methyl-4-morpholino-6-(3-phenyl-1H-pyrazol-1-yl)pyrimidin-2-amine (Example 19, Step 1)(0.100 g, 0.232 mmol) in DMSO (1 mL), was added DIPEA (0.16 mL, 0.929 mmol) and 2,4-difluoropyrimidine (0.05 g, 0.464 mmol) at room temperature under nitrogen atmosphere. The reaction mixture was stirred at room temperature for 4 h. After completion, the reaction mixture was quenched by water and extracted with EtOAc (2×50 mL). The combined organic layer was dried over anhydrous sodium sulfate, filtered and solvent was removed in vacuum to obtain crude product. The resulting crude product was purified by column chromatography. Elution with 8% EtOAc in hexanes afforded N-(2-(1-(2-fluoropyrimidin-4-yl)-1H-pyrazol-4-yl)ethyl)-N-methyl-4-morpholino-6-(3-phenyl-1H-pyrazol-1-yl)pyrimidin-2-amine (Example 20)(0.019 g, 0.036 mmol, 15%) as a white solid.

¹H NMR (400 MHz, DMSO-d6): δ 2.85 (t, 2H), 3.14 (s, 3H), 3.58-3.65 (m, 4H), 3.66-3.72 (m, 4H), 3.86-3.94 (m, 2H), 6.54 (s, 1H), 7.02 (d, 1H), 7.37-7.41 (m, 1H), 7.45-7.48 (m, 2H), 7.79-7.81 (m, 1H), 7.93-7.97 (m, 3H), 8.54 (s, 1H), 8.62 (d, 1H), 8.75 (d, 1H).

¹⁹F NMR (400 MHz, DMSO-d6): δ−45.62 (1F).

LCMS(Method-C): Retention time: 2.301 min, ES(+ve): 527.6 [M+H]⁺

Further elution with 21% EtOAc in hexanes afforded N-(2-(1-(4-fluoropyrimidin-2-yl)-1H-pyrazol-4-yl)ethyl)-N-methyl-4-morpholino-6-(3-phenyl-1H-pyrazol-1-yl)pyrimidin-2-amine (Example 21)(0.025 g, 0.047 mmol, 20%) as a white solid.

¹H NMR (400 MHz, DMSO-d6): δ 2.85 (t, 2H), 3.15 (s, 3H), 3.60-3.65 (m, 4H), 3.66-3.73 (m, 4H), 3.85-3.93 (m, 2H), 6.55 (s, 1H), 7.03 (d, 1H), 7.27-7.29 (m, 1H), 7.37-7.40 (m, 1H), 7.45-7.48 (m, 2H), 7.83 (s, 1H), 7.96-7.98 (m, 2H), 8.52 (s, 1H), 8.62 (d, 1H), 8.89-8.93 (m, 1H). ¹⁹F NMR (400 MHz, DMSO-d6): δ−58.63 (1F).

LCMS(Method-C): Retention time: 2.150 min, ES(+ve): 527.6 [M+H]⁺

### Example 22: N-(2-(1-(2,5-difluoropyrimidin-4-yl)-1H-pyrazol-4-yl)ethyl)-N-methyl-4-morpholino-6-(3-phenyl-1H-pyrazol-1-yl)pyrimidin-2-amine

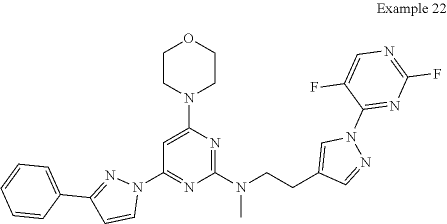 [SMILES: CN(CCc1cnn(-c2nc(F)ncc2F)c1)c1nc(N2CCOCC2)cc(-n2ccc(-c3ccccc3)n2)n1]

Prepared by a method generally analogous to Example 16 using N-(2-(1H-pyrazol-4-yl)ethyl)-N-methyl-4-morpholino-6-(3-phenyl-1H-pyrazol-1-yl)pyrimidin-2-amine (Example 19, Step 1) (0.100 g, 0.232 mmol). The reaction mixture was stirred for 1 h. The crude product was purified by column chromatography. The product was eluted at 10% EtOAc in hexanes to obtain title compound as a white solid (0.065 g, 0.119 mmol, 51%).

¹H NMR (400 MHz, DMSO-d6): δ 2.87 (t, 2H), 3.14 (s, 3H), 3.58-3.65 (m, 4H), 3.67-3.73 (m, 4H), 3.86-3.95 (m, 2H), 6.55 (s, 1H), 7.02 (d, 1H), 7.37-7.40 (m, 1H), 7.47 (t, 2H), 7.95-7.99 (m, 3H), 8.52 (s, 1H), 8.62 (d, 1H), 8.92 (d, 1H). ¹⁹F NMR (400 MHz, DMSO-d6): δ−145.74_−145.82 (1F), −50.90_−50.98 (1F).

LCMS(Method-C): Retention time: 2.237 min, ES(+ve): 545.6 [M+H]⁺

### Example 23: N-(2-(1-(6-fluoropyrimidin-4-yl)-1H-pyrazol-4-yl)ethyl)-N-methyl-4-morpholino-6-(3-phenyl-1H-pyrazol-1-yl)pyrimidin-2-amine

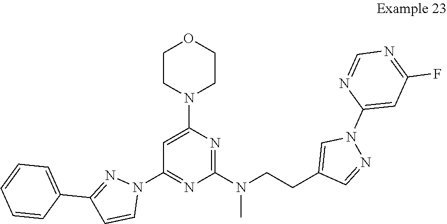 [SMILES: CN(CCc1cnn(-c2cc(F)ncn2)c1)c1nc(N2CCOCC2)cc(-n2ccc(-c3ccccc3)n2)n1]

Prepared the method of Example 20 using 4,6-difluoropyrimidine (0.064 g, 0.557 mmol). The crude product was purified by column chromatography. The product was eluted at 20% EtOAc in hexanes to obtain title compound as white solid (0.080 g, 0.152 mmol, 54%).

¹H NMR (400 MHz, DMSO-d6): δ 2.85 (t, 2H), 3.13 (s, 3H), 3.55-3.71 (m, 8H), 3.82-3.93 (m, 2H), 6.53 (s, 1H), 7.02 (d, 1H), 7.36-7.40 (m, 1H), 7.44-7.48 (m, 2H), 7.53 (s, 1H), 7.92-7.97 (m, 3H), 8.57 (s, 1H), 8.62 (d, 1H), 8.83 (s, 1H).

¹⁹F NMR (400 MHz, DMSO-d6): δ−59.51 (1F).

LCMS(Method-C): Retention time: 2.379 min, ES(+ve): 527.7 [M+H]⁺

### Example 24: N-(2-(1-(6-fluoro-2-methylpyrimidin-4-yl)-1H-pyrazol-4-yl)ethyl)-N-methyl-4-morpholino-6-(3-phenyl-1H-pyrazol-1-yl)pyrimidin-2-amine

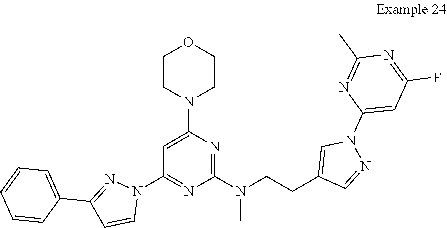 [SMILES: Cc1nc(F)cc(-n2cc(CCN(C)c3nc(N4CCOCC4)cc(-n4ccc(-c5ccccc5)n4)n3)cn2)n1]

Prepared by the method of Example 19, Step 2 using N-(2-(1H-pyrazol-4-yl)ethyl)-N-methyl-4-morpholino-6-(3-phenyl-1H-pyrazol-1-yl)pyrimidin-2-amine (Example 19, Step 1)(0.120 g, 0.278 mmol) and 4,6-difluoro-2-methylpyrimidine (0.072 g, 0.557 mmol). The resulting crude product was purified by column chromatography. The product was eluted at 20% EtOAc in hexane to obtain title compound as white solid (0.083 g, 0.153 mmol, 55%).

¹H NMR (400 MHz, DMSO-d6): δ 2.57 (s, 3H), 2.86 (t, 2H), 3.14 (s, 3H), 3.59-3.64 (m, 4H), 3.66-3.72 (m, 4H), 3.86-3.95 (m, 2H), 6.54 (s, 1H), 7.03 (d, 1H), 7.33 (s, 1H), 7.37-7.41 (m, 1H), 7.45-7.49 (m, 2H), 7.90 (s, 1H), 7.96-7.98 (m, 2H), 8.54 (s, 1H), 8.63 (d, 1H). ¹⁹F NMR (400 MHz, DMSO-d6): δ−60.08 (1F).

LCMS(Method-H): Retention time: 4.823 min, ES(+ve): 541.4 [M+H]⁺

### Example 25: N-(2-(1-(6-chloropyrimidin-4-yl)-1H-pyrazol-4-yl)ethyl)-N-methyl-4-morpholino-6-(3-phenyl-1H-pyrazol-1-yl)pyrimidin-2-amine

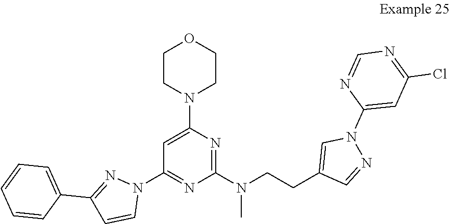 [SMILES: CN(CCc1cnn(-c2cc(Cl)ncn2)c1)c1nc(N2CCOCC2)cc(-n2ccc(-c3ccccc3)n2)n1]

Prepared by a method generally analogous to Example 15 using N-(2-(1H-pyrazol-4-yl)ethyl)-N-methyl-4-morpholino-6-(3-phenyl-1H-pyrazol-1-yl)pyrimidin-2-amine (Example 19, Step 1) (0.100 g, 0.232 mmol) and 4,6-dichloropyrimidine (0.069 g, 0.464 mmol). The crude product was purified by column chromatography. The desired product was eluted at 15% EtOAc in hexanes to obtain title compound as off-white solid. (0.035 g, 0.064 mmol, 27%).

¹H NMR (400 MHz, DMSO-d6): δ 2.86 (t, 2H), 3.14 (s, 3H), 3.60-3.64 (m, 4H), 3.66-3.72 (m, 4H), 3.85-3.95 (m, 2H), 6.54 (s, 1H), 7.02 (d, 1H), 7.37-7.41 (m, 1H), 7.47 (t, 2H), 7.88-7.98 (m, 4H), 8.57 (s, 1H), 8.62 (d, 1H), 8.90 (d, 1H).

LCMS(Method-C): Retention time: 2.421 min, ES(+ve): 543.1/545.1 [M+H]⁺

### Example 26: N-(2-(1-(2,5-difluoropyrimidin-4-yl)-1H-pyrazol-4-yl)ethyl)-N-methyl-4-morpholino-6-(1H-pyrazol-1-yl)pyrimidin-2-amine and Example 27: N-(2-(1-(4,5-difluoropyrimidin-2-yl)-1H-pyrazol-4-yl)ethyl)-N-methyl-4-morpholino-6-(1H-pyrazol-1-yl)pyrimidin-2-amine

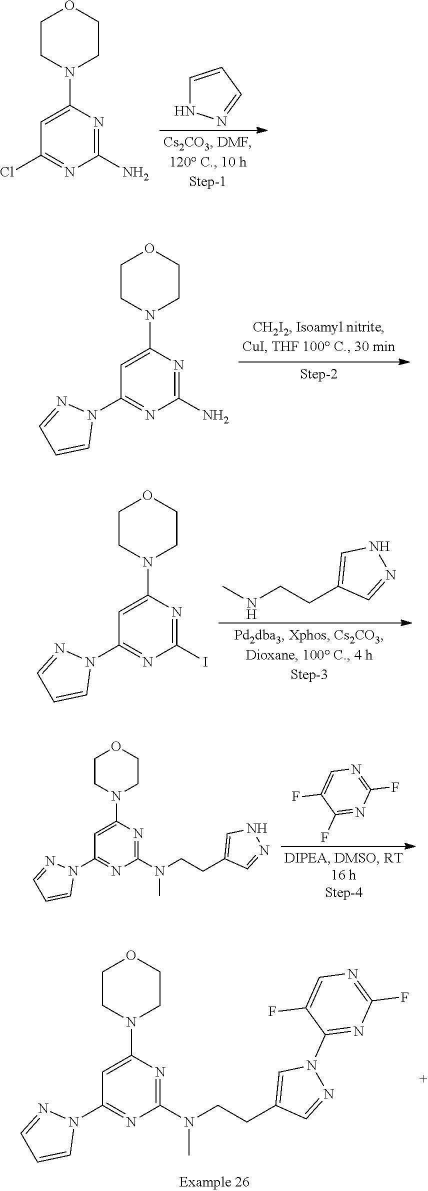

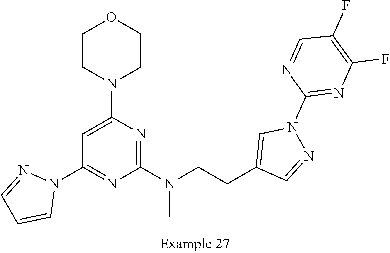 [SMILES: CN(CCc1cnn(-c2ncc(F)c(F)n2)c1)c1nc(N2CCOCC2)cc(-n2cccn2)n1]

### Step 1: Synthesis of 4-morpholino-6-(1H-pyrazol-1-yl)pyrimidin-2-amine

To a stirred solution of 4-chloro-6-morpholinopyrimidin-2-amine (10.0 g, 46.587 mmol) in DMF (100 mL), was added Cs₂CO₃ (30.28 g, 93.174 mmol) and 1H-pyrazole (8.10 g, 51.246 mmol) at room temperature under nitrogen atmosphere. The reaction mixture was heated at 120° C. and stirred for 10 h. After completion, the reaction mixture was quenched with water (300 mL) and extracted with EtOAc (3×400 mL). The combined organic phase was dried over sodium sulfate and evaporated under reduced pressure to get crude product. The crude was purified by silica gel flash chromatography. Pure compound was eluted at a gradient of 20-30% EtOAc in hexanes to obtain title compound as a solid (5.7 g, 23.145 mmol, 49%).

LCMS(Method-C): Retention time: 1.373 min, ES(+ve): 247.3 [M+H]⁺

### Step 2: Synthesis of 4-(2-iodo-6-(1H-pyrazol-1-yl)pyrimidin-4-yl)morpholine

To a solution of 4-morpholino-6-(1H-pyrazol-1-yl)pyrimidin-2-amine (1.00 g, 4.060 mmol) in THF (10 mL) were added CuI (0.85 g, 4.466), diiodomethane (5.43 g, 20.302 mmol) and isoamyl nitrite (0.95 g, 8.121 mmol) under nitrogen atmosphere and the mixture was stirred at 100° C. for 30 min. After completion, the reaction solvent was evaporated under reduced pressure to obtain crude compound. The crude was purified by silica gel flash chromatography. Pure compound was eluted at a gradient of 10-15% EtOAc in hexanes to obtain title compound as an off-white solid (0.430 g, 1.203 mmol, 29%).

¹H NMR (400 MHz, CDCl₃): δ 3.62-3.83 (m, 8H), 6.44-6.46 (m, 1H), 7.04 (s, 1H), 7.74 (d, 1H), 8.49 (d, 1H).

LCMS(Method-C): Retention time: 1.825 min, ES(+ve): 358.2 [M+H]⁺

### Step 3: Synthesis of N-(2-(1H-pyrazol-4-yl)ethyl)-N-methyl-4-morpholino-6-(1H-pyrazol-1-yl)pyrimidin-2-amine

Cs₂CO₃ (1.45 g, 4.479 mmol) was added to a stirred solution of 4-(2-iodo-6-(1H-pyrazol-1-yl)pyrimidin-4-yl)morpholine (0.40 g, 1.119 mmol) and N-methyl-2-(1H-pyrazol-4-yl) ethan-1-amine (0.21 g, 1.679 mmol) in dioxane (4.0 mL) at room temperature under nitrogen atmosphere. The reaction mixture was purged with nitrogen gas for 10 min. Pd₂(dba)₃ (0.153 g, 0.167 mmol) and XPhos (CAS: 564483-18-7)(0.266 g, 0.559 mmol) were added and the reaction mixture was heated at 100° C. for 4 h. After completion, the reaction mixture was diluted with water (10 mL) and the aqueous layer was extracted with EtOAc (3×20 mL). The combined organic phases were dried over sodium sulfate and evaporated under reduced pressure. The crude residue was purified by silica gel flash chromatography and pure compound was eluted at 2-4% MeOH in DCM to obtain title compound as a yellow solid (0.23 g, 0.648 mmol, 57%).

¹H NMR (400 MHz, DMSO-d6): δ 2.66-2.73 (m, 2H), 3.09 (s, 3H), 3.50-3.80 (m, 10H), 6.45 (s, 1H), 6.52 (s, 1H), 7.30-7.65 (m, 2H), 7.77 (s, 1H), 8.53 (s, 1H), 12.55 (s, 1H).

LCMS(Method-H): Retention time: 2.838 min, ES(+ve): 355.2 [M+H]⁺

### Step 4: Synthesis of N-(2-(1-(2,5-difluoropyrimidin-4-yl)-1H-pyrazol-4-yl)ethyl)-N-methyl-4-morpholino-6-(1H-pyrazol-1-yl)pyrimidin-2-amine and N-(2-(1-(4,5-difluoropyrimidin-2-yl)-1H-pyrazol-4-yl)ethyl)-N-methyl-4-morpholino-6-(1H-pyrazol-1-yl)pyrimidin-2-amine

To a stirred solution of N-(2-(1H-pyrazol-4-yl)ethyl)-N-methyl-4-morpholino-6-(1H-pyrazol-1-yl)pyrimidin-2-amine (0.100 g, 0.282 mmol) in DMSO (1 mL), was added DIPEA (0.19 mL, 1.128 mmol) and 2,4,5-trifluoropyrimidine (0.075 g, 0.564 mmol) at room temperature under nitrogen atmosphere. The reaction mixture was stirred at room temperature for 16 h. After completion, the reaction mixture was quenched by water (5 mL) and extracted with EtOAc (2×30 mL). The combined organic layer was dried over anhydrous sodium sulfate, filtered and solvent was removed in vacuum to obtain crude product. The resulting crude product was purified by column chromatography. Elution with 10% EtOAc in hexanes afforded N-(2-(1-(2,5-difluoropyrimidin-4-yl)-1H-pyrazol-4-yl)ethyl)-N-methyl-4-morpholino-6-(1H-pyrazol-1-yl)pyrimidin-2-amine as a white solid (0.048 g, 0.102 mmol, 36%).

¹H NMR (400 MHz, DMSO-d6): δ 2.85 (t, 2H), 3.11 (s, 3H), 3.55-3.60 (m, 4H), 3.63-3.68 (m, 4H), 3.83-3.91 (m, 2H), 6.43 (s, 1H), 6.50 (s, 1H), 7.75 (s, 1H), 7.96 (s, 1H), 8.49 (s, 1H), 8.55 (d, 1H), 8.92 (d, 1H).

¹⁹F NMR (400 MHz, DMSO-d6): δ−145.75_−145.83 (1F), −50.92_−50.99 (1F).

LCMS(Method-H): Retention time: 3.539 min, ES(+ve): 469.0 [M+H]⁺

Further elution with 20% EtOAc in hexane afforded N-(2-(1-(4,5-difluoropyrimidin-2-yl)-1H-pyrazol-4-yl)ethyl)-N-methyl-4-morpholino-6-(1H-pyrazol-1-yl)pyrimidin-2-amine as an off-white solid (0.009 g, 0.019 mmol, 7%).

¹H NMR (400 MHz, DMSO-d6): δ 2.82 (t, 2H), 3.11 (s, 3H), 3.54-3.60 (m, 4H), 3.63-3.68 (m, 4H), 3.82-3.88 (m, 2H), 6.43 (s, 1H), 6.51 (s, 1H), 7.76 (s, 1H), 7.79 (s, 1H), 8.44 (s, 1H), 8.55 (s, 1H), 9.02-9.08 (m, 1H).

¹⁹F NMR (400 MHz, DMSO-d6): δ−158.34_−158.41 (1F), −78.41_−78.48 (1F).

LCMS(Method-H): Retention time: 3.436 min, ES(+ve): 469.0 [M+H]⁺

### Example 28: N-(2-(1-(2-fluoropyrimidin-4-yl)-1H-pyrazol-4-yl)ethyl)-N-methyl-4-morpholino-6-(1H-pyrazol-1-yl)pyrimidin-2-amine and Example 29: N-(2-(1-(4-fluoropyrimidin-2-yl)-1H-pyrazol-4-yl)ethyl)-N-methyl-4-morpholino-6-(1H-pyrazol-1-yl)pyrimidin-2-amine

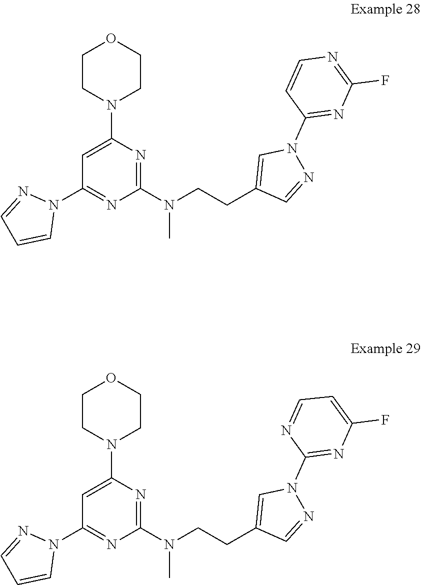 [SMILES: CN(CCc1cnn(-c2ccnc(F)n2)c1)c1nc(N2CCOCC2)cc(-n2cccn2)n1 ; CN(CCc1cnn(-c2nccc(F)n2)c1)c1nc(N2CCOCC2)cc(-n2cccn2)n1]

Prepared by a method generally analogous to Examples 20 and 21 using N-(2-(1H-pyrazol-4-yl)ethyl)-N-methyl-4-morpholino-6-(1H-pyrazol-1-yl)pyrimidin-2-amine (Examples 26 and 27, Step 3)(0.200 g, 0.564 mmol). The reaction mixture was stirred at room temperature for 16 h. The crude product was purified by column chromatography. Elution with 20% EtOAc in hexanes afforded N-(2-(1-(2-fluoropyrimidin-4-yl)-1H-pyrazol-4-yl)ethyl)-N-methyl-4-morpholino-6-(1H-pyrazol-1-yl)pyrimidin-2-amine (Example 28)(0.055 g, 0.122 mmol, 21%) as an off-white solid.

¹H NMR (400 MHz, DMSO-d6): δ 2.84 (t, 2H), 3.12 (s, 3H), 3.54-3.61 (m, 4H), 3.63-3.71 (m, 4H), 3.85-3.89 (m, 2H), 6.43 (s, 1H), 6.50-6.51 (m, 1H), 7.76-7.81 (m, 2H), 7.92 (s, 1H), 8.53-8.56 (m, 2H), 8.76-8.78 (m, 1H).

¹⁹F NMR (400 MHz, DMSO-d6): δ−45.63 (1F).

LCMS(Method-C): Retention time: 1.929 min, ES(+ve): 451.4 [M+H]⁺

Further elution with 2% MeOH in DCM afforded N-(2-(1-(4-fluoropyrimidin-2-yl)-1H-pyrazol-4-yl)ethyl)-N-methyl-4-morpholino-6-(1H-pyrazol-1-yl)pyrimidin-2-amine (Example 29)(0.035 g, 0.0776 mmol, 14%) as an off-white solid.

¹H NMR (400 MHz, DMSO-d6): δ 2.83 (t, 2H), 3.11 (s, 3H), 3.53-3.61 (m, 4H), 3.62-3.69 (m, 4H), 3.72-3.91 (m, 2H), 6.43 (s, 1H), 6.50 (s, 1H), 7.27-7.29 (m, 1H), 7.76-7.80 (m, 2H), 8.50-8.55 (m, 2H), 8.88-8.92 (m, 1H).

¹⁹F NMR (400 MHz, DMSO-d6): δ−58.64 (1F).

LCMS(Method-C): Retention time: 1.770 min, ES(+ve): 451.4 [M+H]⁺

### Example 30: N-(2-(1-(6-fluoropyrimidin-4-yl)-1H-pyrazol-4-yl)ethyl)-N-methyl-4-morpholino-6-(1H-pyrazol-1-yl)pyrimidin-2-amine

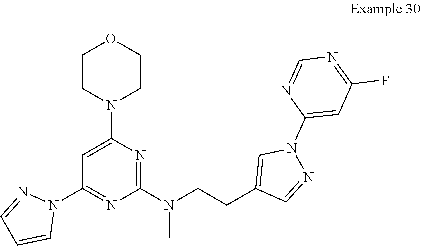 [SMILES: CN(CCc1cnn(-c2cc(F)ncn2)c1)c1nc(N2CCOCC2)cc(-n2cccn2)n1]

Prepared by a method generally analogous to Example 17 using N-(2-(1H-pyrazol-4-yl)ethyl)-N-methyl-4-morpholino-6-(1H-pyrazol-1-yl)pyrimidin-2-amine (Examples 26 and 27, Step 3) (0.120 g, 0.338 mmol). The reaction mixture was stirred at room temperature for 5 h. The crude product was purified by column chromatography. The product was eluted at 25% EtOAc in hexanes to obtain title compound as a white solid (0.055 g, 0.122 mmol, 36%).

¹H NMR (400 MHz, DMSO-d6): δ 2.85 (t, 2H), 3.13 (s, 3H), 3.55-3.62 (m, 4H), 3.64-3.70 (m, 4H), 3.83-3.92 (m, 2H), 6.44 (s, 1H), 6.51-6.52 (m, 1H), 7.54 (s, 1H), 7.77 (d, H), 7.91 (s, 1H), 8.56 (d, 2H), 8.84 (d, 1H). ¹⁹F NMR (400 MHz, DMSO-d6): δ−59.66 (1F).

LCMS(Method-C): Retention time: 1.977 min, ES(+ve): 451.4 [M+H]⁺

### Example 31: N-(2-(1-(2-fluoropyrimidin-4-yl)-1H-pyrazol-4-yl)ethyl)-4-morpholino-6-(3-phenyl-1H-pyrazol-1-yl)pyrimidin-2-amine and Example 32: N-(2-(1-(4-fluoropyrimidin-2-yl)-1H-pyrazol-4-yl)ethyl)-4-morpholino-6-(3-phenyl-1H-pyrazol-1-yl)pyrimidin-2-amine

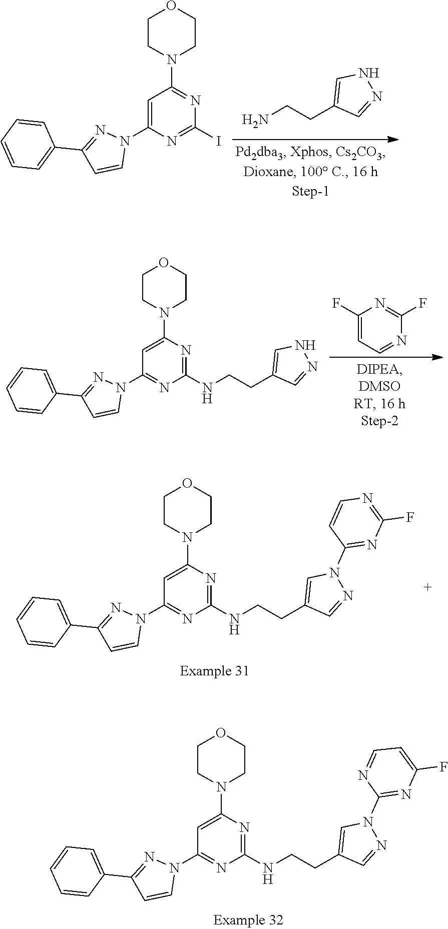

### Step 1: Synthesis of N-(2-(1H-pyrazol-4-yl)ethyl)-4-morpholino-6-(3-phenyl-1H-pyrazol-1-yl)pyrimidin-2-amine

Cs₂CO₃ (0.90 g, 2.769 mmol) was added to a stirred solution of 4-(2-iodo-6-(3-phenyl-1H-pyrazol-1-yl)pyrimidin-4-yl)morpholine (WO2021163727 A1)(0.30 g, 0.692 mmol) and 2-(1H-pyrazol-4-yl) ethan-1-amine (0.154 g, 1.384 mmol) in dioxane (3.0 mL) at room temperature under nitrogen atmosphere. The reaction mixture was purged with nitrogen gas for 10 min. Pd₂(dba)₃ (0.095 g, 0.103 mmol) and XPhos (CAS: 564483-18-7)(0.165 g, 0.346 mmol) were added and the reaction mixture was heated at 100° C. for 16 h. After completion, the reaction mixture was diluted with water (10 mL) and aqueous layer was extracted with EtOAc (3×30 mL). The combined organic phases were dried over sodium sulfate and evaporated under reduced pressure. The crude residue was purified by silica gel flash chromatography and pure compound was eluted at 2-4% MeOH in DCM to obtain title compound as a yellow solid (0.12 g, 0.288 mmol, 42%).

¹H NMR (400 MHz, DMSO-d6): δ 2.66-2.72 (m, 2H), 3.37-3.39 (m, 2H), 3.56-3.73 (m, 8H), 6.56 (s, 1H), 6.92-6.99 (m, 1H), 7.02-7.03 (m, 1H), 7.36-7.38 (m, 2H), 7.44-7.47 (m, 2H), 7.52-7.57 (m, 1H), 7.95-7.97 (m, 2H), 8.47-8.54 (m, 1H), 12.54 (s, 1H).

LCMS(Method-C): Retention time: 1.695 min, ES(+ve): 417.4 [M+H]⁺

### Step 2: Synthesis of N-(2-(1-(2-fluoropyrimidin-4-yl)-1H-pyrazol-4-yl)ethyl)-4-morpholino-6-(3-phenyl-1H-pyrazol-1-yl)pyrimidin-2-amine and N-(2-(1-(4-fluoropyrimidin-2-yl)-1H-pyrazol-4-yl)ethyl)-4-morpholino-6-(3-phenyl-1H-pyrazol-1-yl)pyrimidin-2-amine

Prepared by a method generally analogous to Examples 20 and 21 using N-(2-(1H-pyrazol-4-yl)ethyl)-4-morpholino-6-(3-phenyl-1H-pyrazol-1-yl)pyrimidin-2-amine (0.120 g, 0.288 mmol). The reaction mixture was stirred at room temperature for 16 h. The resulting crude product was purified by column chromatography. Elution with 20% EtOAc in hexanes afforded N-(2-(1-(2-fluoropyrimidin-4-yl)-1H-pyrazol-4-yl)ethyl)-4-morpholino-6-(3-phenyl-1H-pyrazol-1-yl)pyrimidin-2-amine (Example 31)(0.008 g, 0.016 mmol, 5%) as an off-white solid.

¹H NMR (400 MHz, DMSO-d6): δ 2.82 (t, 2H), 3.53-3.72 (m, 10H), 6.55 (s, 1H), 7.01-7.12 (m, 2H), 7.36-7.39 (m, 1H), 7.43-7.47 (m, 2H), 7.81-7.83 (m, 1H), 7.94-7.96 (m, 3H), 8.43-8.65 (m, 2H), 8.76-8.77 (m, 1H).

¹⁹F NMR (400 MHz, DMSO-d6): δ−45.57 (1F).

LCMS(Method-C): Retention time: 1.980 min, ES(+ve): 513.2 [M+H]⁺

Further elution with 70% EtOAc in hexanes afforded N-(2-(1-(4-fluoropyrimidin-2-yl)-1H-pyrazol-4-yl)ethyl)-4-morpholino-6-(3-phenyl-1H-pyrazol-1-yl)pyrimidin-2-amine (Example 32)(0.050 g, 0.097 mmol, 34%) as an off-white solid.

¹H NMR (400 MHz, DMSO-d6): δ 2.81 (t, 2H), 3.52-3.72 (m, 10H), 6.57 (s, 1H), 7.02-7.08 (m, 2H), 7.28-7.32 (m, 1H), 7.38-7.40 (m, 1H), 7.45-7.48 (m, 2H), 7.84 (s, 1H), 7.95-7.97 (m, 2H), 8.45-8.65 (m, 2H), 8.90-8.95 (m, 1H).

¹⁹F NMR (400 MHz, DMSO-d6): δ−58.63 (1F).

LCMS(Method-C): Retention time: 1.879 min, ES(+ve): 513.5 [M+H]⁺

### Example 33: N-(2-(1-(2,5-dichloropyrimidin-4-yl)-1H-pyrazol-4-yl)ethyl)-N-methyl-4-morpholino-6-(3-phenyl-1H-pyrazol-1-yl)pyrimidin-2-amine and Example 34: N-(2-(1-(4,5-dichloropyrimidin-2-yl)-1H-pyrazol-4-yl)ethyl)-N-methyl-4-morpholino-6-(3-phenyl-1H-pyrazol-1-yl)pyrimidin-2-amine

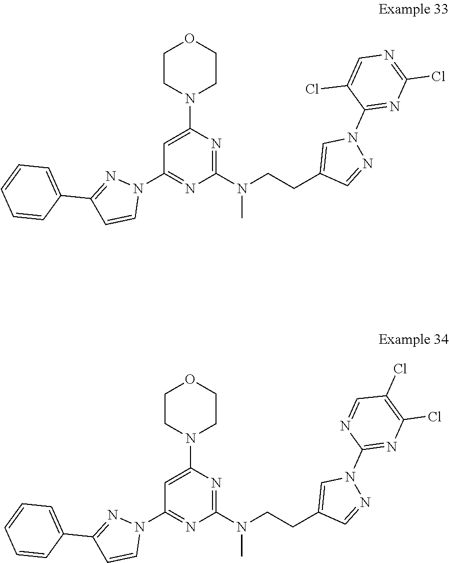 [SMILES: CN(CCc1cnn(-c2nc(Cl)ncc2Cl)c1)c1nc(N2CCOCC2)cc(-n2ccc(-c3ccccc3)n2)n1 ; CN(CCc1cnn(-c2ncc(Cl)c(Cl)n2)c1)c1nc(N2CCOCC2)cc(-n2ccc(-c3ccccc3)n2)n1]

Prepared by a method generally analogous to Examples 20 and 21 using 2,4,5-trichloropyrimidine (0.170 g, 0.929 mmol). The reaction mixture was stirred at room temperature for 16 h. The resulting crude product was purified by column chromatography. Elution with 15% EtOAc in hexanes afforded N-(2-(1-(2,5-dichloropyrimidin-4-yl)-1H-pyrazol-4-yl)ethyl)-N-methyl-4-morpholino-6-(3-phenyl-1H-pyrazol-1-yl)pyrimidin-2-amine (Example 33)(0.050 g, 0.086 mmol, 18%) as a pale yellow solid.

¹H NMR (400 MHz, DMSO-d6): δ 2.82-2.90 (m, 2H), 3.14 (s, 3H), 3.58-3.72 (m, 8H), 3.82-3.93 (m, 2H), 6.54 (s, 1H), 7.02 (s, 1H), 7.38-7.39 (m, 1H), 7.44-7.48 (m, 2H), 7.95-7.96 (m, 3H), 8.47 (s, 1H), 8.60-8.63 (m, 1H), 8.94 (s, 1H).

LCMS(Method-H): Retention time: 4.635 min, 577.4/579.3 [M+H]⁺

Further elution with 30% EtOAc in hexanes afforded N-(2-(1-(4,5-dichloropyrimidin-2-yl)-1H-pyrazol-4-yl)ethyl)-N-methyl-4-morpholino-6-(3-phenyl-1H-pyrazol-1-yl)pyrimidin-2-amine (Example 34)(0.050 g, 0.086 mmol, 18%) as a pale yellow solid.

¹H NMR (400 MHz, DMSO-d6): δ 2.80-2.88 (m, 2H), 3.13 (s, 3H), 3.54-3.72 (m, 8H), 3.80-3.92 (m, 2H), 6.53 (s, 1H), 7.02 (s, 1H), 7.38-7.39 (m, 1H), 7.44-7.46 (m, 2H), 7.84 (m, 1H), 7.95-7.97 (m, 2H), 8.49 (s, 1H), 8.59-8.64 (m, 1H), 8.99 (s, 1H).

LCMS(Method-H): Retention time: 4.430 min, ES(+ve): 577.1/579.2 [M+H]⁺

### Example 35: N-(2-(1-(5-chloro-2-fluoropyrimidin-4-yl)-1H-pyrazol-4-yl)ethyl)-N-methyl-4-morpholino-6-(3-phenyl-1H-pyrazol-1-yl)pyrimidin-2-amine and Example 36: N-(2-(1-(5-chloro-4-fluoropyrimidin-2-yl)-1H-pyrazol-4-yl)ethyl)-N-methyl-4-morpholino-6-(3-phenyl-1H-pyrazol-1-yl)pyrimidin-2-amine

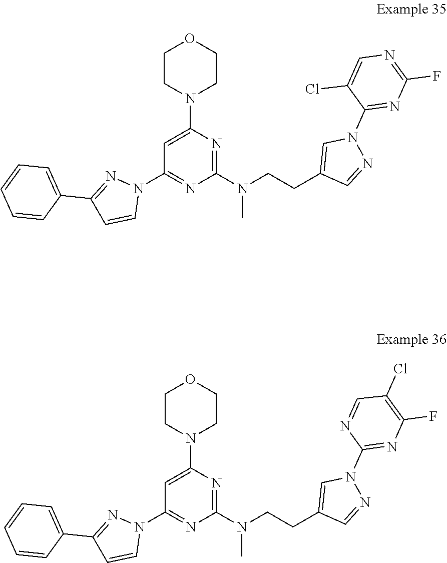 [SMILES: CN(CCc1cnn(-c2nc(F)ncc2Cl)c1)c1nc(N2CCOCC2)cc(-n2ccc(-c3ccccc3)n2)n1 ; CN(CCc1cnn(-c2ncc(Cl)c(F)n2)c1)c1nc(N2CCOCC2)cc(-n2ccc(-c3ccccc3)n2)n1]

Prepared by a method generally analogous to Examples 20 and 21 using 5-chloro-2,4-difluoropyrimidine (0.105 g, 0.697 mmol). The reaction mixture was stirred at room temperature for 1 h. The resulting crude product was purified by column chromatography. Elution with 20% EtOAc in hexanes afforded N-(2-(1-(5-chloro-2-fluoropyrimidin-4-yl)-1H-pyrazol-4-yl)ethyl)-N-methyl-4-morpholino-6-(3-phenyl-1H-pyrazol-1-yl)pyrimidin-2-amine (Example 35)(0.063 g, 0.112 mmol, 32%) as an off-white solid.

¹H NMR (400 MHz, DMSO-d6): δ 2.87 (t, 2H), 3.15 (s, 3H), 3.60-3.65 (m, 4H), 3.66-3.72 (m, 4H), 3.85-3.93 (m, 2H), 6.55 (s, 1H), 7.02 (d, 1H), 7.37-7.40 (m, 1H), 7.45-7.48 (m, 2H), 7.95-7.97 (m, 3H), 8.51 (s, 1H), 8.62 (d, 1H), 8.97 (s, 1H).

¹⁹F NMR (400 MHz, DMSO-d6): δ−49.22 (1F).

LCMS(Method-H): Retention time: 4.419 min, ES(+ve): 561.2/563.0 [M+H]⁺

Further elution with 35% EtOAc in hexanes afforded N-(2-(1-(5-chloro-4-fluoropyrimidin-2-yl)-1H-pyrazol-4-yl)ethyl)-N-methyl-4-morpholino-6-(3-phenyl-1H-pyrazol-1-yl)pyrimidin-2-amine (Example 36)(0.015 g, 0.027 mmol, 8%) as an off-white solid.

¹H NMR (400 MHz, DMSO-d6): δ 2.85 (t, 2H), 3.14 (s, 3H), 3.60-3.65 (m, 4H), 3.67-3.72 (m, 4H), 3.84-3.92 (m, 2H), 6.55 (s, 1H), 7.03 (d, 1H), 7.37-7.40 (m, 1H), 7.45-7.48 (m, 2H), 7.85 (s, 1H), 7.95-7.98 (m, 2H), 8.49 (s, 1H), 8.62 (d, 1H), 9.03-9.06 (m, 1H).

¹⁹F NMR (400 MHz, DMSO-d6): δ−63.26 (1F).

LCMS(Method-H): Retention time: 4.218 min, ES(+ve): 561.2/563.1 [M+H]⁺

### Example 37: N-(2-(1-(6-chloro-2-fluoropyrimidin-4-yl)-1H-pyrazol-4-yl)ethyl)-N-methyl-4-morpholino-6-(3-phenyl-1H-pyrazol-1-yl)pyrimidin-2-amine

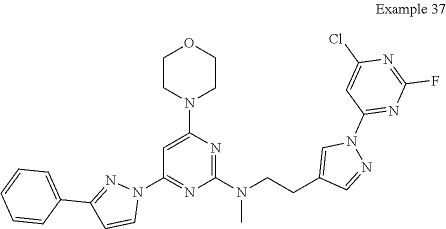 [SMILES: CN(CCc1cnn(-c2cc(Cl)nc(F)n2)c1)c1nc(N2CCOCC2)cc(-n2ccc(-c3ccccc3)n2)n1]

Prepared by the method of Example 19, Step 2 using 4,6-dichloro-2-fluoropyrimidine (0.116 g, 0.697 mmol). The resulting crude product was purified by column chromatography. The product was eluted at 20% EtOAc in hexanes to obtain title compound as an off-white solid (0.018 g, 0.032 mmol, 9%).

¹H NMR (400 MHz, DMSO-d6): δ 2.84 (t, 2H), 3.12 (s, 3H), 3.56-3.63 (m, 4H), 3.64-3.71 (m, 4H), 3.83-3.92 (m, 2H), 6.53 (s, 1H), 7.01 (d, 1H), 7.35-7.39 (m, 1H), 7.44-7.47 (m, 2H), 7.84 (s, 1H), 7.94-7.96 (m, 3H), 8.52 (s, 1H), 8.61 (d, 1H).

¹⁹F NMR (400 MHz, DMSO-d6): δ−44.60 (1F).

LCMS(Method-C): Retention time: 2.486 min, ES(+ve): 561.4/563.4 [M+H]⁺

### Example 38: N-(2-(1-(4,6-dichloropyrimidin-2-yl)-1H-pyrazol-4-yl)ethyl)-N-methyl-4-morpholino-6-(3-phenyl-1H-pyrazol-1-yl)pyrimidin-2-amine and Example 39: N-(2-(1-(2,6-dichloropyrimidin-4-yl)-1H-pyrazol-4-yl)ethyl)-N-methyl-4-morpholino-6-(3-phenyl-1H-pyrazol-1-yl)pyrimidin-2-amine

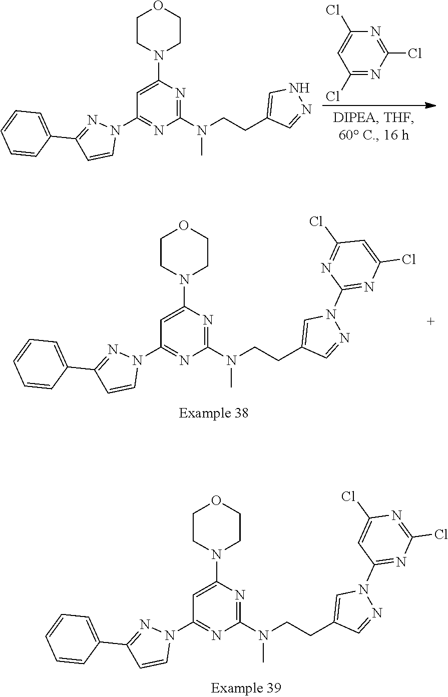

To a stirred solution of N-(2-(1H-pyrazol-4-yl)ethyl)-N-methyl-4-morpholino-6-(3-phenyl-1H-pyrazol-1-yl)pyrimidin-2-amine (Example 19, Step 1)(0.150 g, 0.348 mmol) in THF (1.5 mL), was added DIPEA (0.15 mL, 0.861 mmol) and 2,4,6-trichloropyrimidine (0.15 mL, 0.512 mmol) at room temperature under nitrogen atmosphere. The reaction mixture was stirred at 60° C. for 16 h. After completion, the reaction mixture was quenched with water and extracted with EtOAc (2×50 mL). The combined organic layer was dried over anhydrous sodium sulfate, filtered and solvent was removed in vacuum to obtain crude product. The resulting crude was purified by reverse phase column chromatography. Elution with 45% MeCN in water afforded N-(2-(1-(4,6-dichloropyrimidin-2-yl)-1H-pyrazol-4-yl)ethyl)-N-methyl-4-morpholino-6-(3-phenyl-1H-pyrazol-1-yl)pyrimidin-2-amine (Example 38)(0.020 g, 0.034 mmol, 10%) as an off-white solid.

¹H NMR (400 MHz, DMSO-d6): δ 2.80-2.87 (m, 2H), 3.13 (s, 3H), 3.58-3.64 (m, 4H), 3.65-3.71 (m, 4H), 3.84-3.93 (m, 2H), 6.54 (s, 1H), 7.01 (d, 1H), 7.35-7.41 (m, 1H), 7.44-7.47 (m, 2H), 7.86 (s, 2H), 7.94-7.96 (m, 2H), 8.47 (s, 1H), 8.61-8.62 (m, 1H).

LCMS(Method-C): Retention time: 2.381 min, ES(+ve): 577.2/579.2 [M+H]⁺

Further elution with 56% MeCN in water afforded N-(2-(1-(2,6-dichloropyrimidin-4-yl)-1H-pyrazol-4-yl)ethyl)-N-methyl-4-morpholino-6-(3-phenyl-1H-pyrazol-1-yl)pyrimidin-2-amine

(Example 39)(0.020 g, 0.034 mmol, 10%) as an off-white solid.

¹H NMR (400 MHz, DMSO-d6): δ 2.81-2.89 (m, 2H), 3.13 (s, 3H), 3.57-3.64 (m, 4H), 3.65-3.71 (m, 4H), 3.84-3.92 (m, 2H), 6.53 (s, 1H), 7.01 (d, 1H), 7.35-7.41 (m, 1H), 7.43-7.47 (m, 2H), 7.86 (s, 1H), 7.94-7.96 (m, 3H), 8.52 (s, 1H), 8.61-8.62 (m, 1H).

LCMS(Method-C): Retention time: 2.603 min, ES(+ve): 577.2/579.3 [M+H]⁺

### Example 40: N-(2-(1-(4,5-difluoropyrimidin-2-yl)-1H-pyrazol-4-yl)ethyl)-N-methyl-4-morpholino-6-(3-phenyl-1H-pyrazol-1-yl)pyrimidin-2-amine

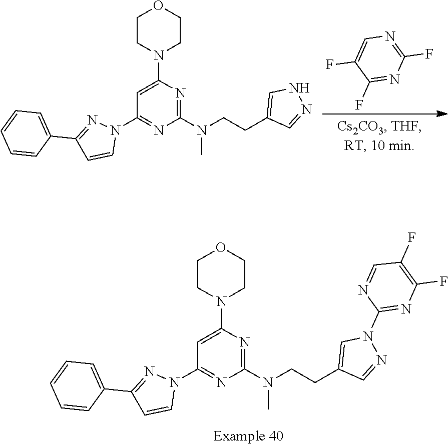 [SMILES: CN(CCc1cn[nH]c1)c1nc(N2CCOCC2)cc(-n2ccc(-c3ccccc3)n2)n1 ; Fc1ncc(F)c(F)n1 ; CN(CCc1cnn(-c2ncc(F)c(F)n2)c1)c1nc(N2CCOCC2)cc(-n2ccc(-c3ccccc3)n2)n1]

To a stirred mixture of N-(2-(1H-pyrazol-4-yl)ethyl)-N-methyl-4-morpholino-6-(3-phenyl-1H-pyrazol-1-yl)pyrimidin-2-amine (Example 19, Step 1)(0.200 g, 0.464 mmol) and Cs₂CO₃ (0.408 g, 1.254 mmol) was added a solution of 2,4,5-trifluoropyrimidine (0.199 g, 1.486 mmol) in THF (2 mL) at room temperature under nitrogen atmosphere. The reaction mixture was stirred at room temperature for 10 min. After completion, the reaction mixture was quenched with water (100 mL) and extracted with EtOAc (2×100 mL). The combined organic layer was dried over anhydrous sodium sulfate, filtered and solvent was removed in vacuum to obtain crude product. The resulting crude product was purified by column chromatography. The product was eluted at 30% EtOAc in hexanes to obtain title compound as an off-white solid (0.027 g, 0.049 mmol, 10%).

¹H NMR (400 MHz, DMSO-d6): δ 2.83 (t, 2H), 3.13 (s, 3H), 3.53-3.75 (m, 8H), 3.82-3.93 (m, 2H), 6.54 (s, 1H), 7.02 (d, 1H), 7.34-7.41 (m, 1H), 7.44-7.48 (m, 2H), 7.81 (s, 1H), 7.95-7.97 (m, 2H), 8.45 (s, 1H), 8.61 (d, 1H), 9.03-9.06 (m, 1H).

¹⁹F NMR (400 MHz, DMSO-d6): δ−158.41_−158.34 (1F), −78.47_−78.40 (1F).

LCMS(Method-H): Retention time: 4.121 min, ES(+ve): 545.2 [M+H]⁺

### Example 41: N-(2-(1-(6-fluoro-5-methylpyrimidin-4-yl)-1H-pyrazol-4-yl)ethyl)-N-methyl-4-morpholino-6-(3-phenyl-1H-pyrazol-1-yl)pyrimidin-2-amine

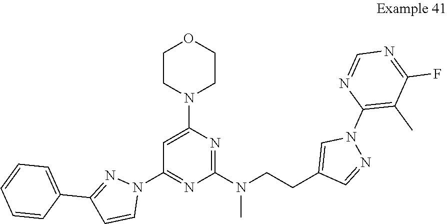 [SMILES: Cc1c(F)ncnc1-n1cc(CCN(C)c2nc(N3CCOCC3)cc(-n3ccc(-c4ccccc4)n3)n2)cn1]

Prepared by the method of Example 16 using N-(2-(1H-pyrazol-4-yl)ethyl)-N-methyl-4-morpholino-6-(3-phenyl-1H-pyrazol-1-yl)pyrimidin-2-amine (Example 19, Step 1)(0.120 g, 0.278 mmol) and 4,6-difluoro-5-methylpyrimidine (0.072 g, 0.557 mmol). The resulting crude product was purified by column chromatography. The product was eluted at 15% EtOAc in hexanes to obtain title compound as an off-white solid (0.043 g, 0.079 mmol, 28%)

¹H NMR (400 MHz, DMSO-d6): δ 2.43 (s, 3H), 2.81-2.88 (m, 2H), 3.15 (s, 3H), 3.58-3.64 (m, 4H), 3.65-3.72 (m, 4H), 3.83-3.92 (m, 2H), 6.53 (s, 1H), 7.01 (s, 1H), 7.35-7.42 (m, 1H), 7.44-7.48 (m, 2H), 7.88 (s, 1H), 7.95-7.96 (m, 2H), 8.55 (s, 1H), 8.61 (s, 1H), 8.66 (s, 1H).

¹⁹F NMR (400 MHz, DMSO-d6): δ−64.21 (1F).

LCMS(Method-H): Retention time: 4.811 min, ES(+ve): 541.4 [M+H]⁺

### Example 42: N-(2-(1-(2-fluoropyrimidin-4-yl)-1H-pyrazol-3-yl)ethyl)-N-methyl-4-morpholino-6-(3-phenyl-1H-pyrazol-1-yl)pyrimidin-2-amine and Example 43: N-(2-(1-(4-fluoropyrimidin-2-yl)-1H-pyrazol-5-yl)ethyl)-N-methyl-4-morpholino-6-(3-phenyl-1H-pyrazol-1-yl)pyrimidin-2-amine

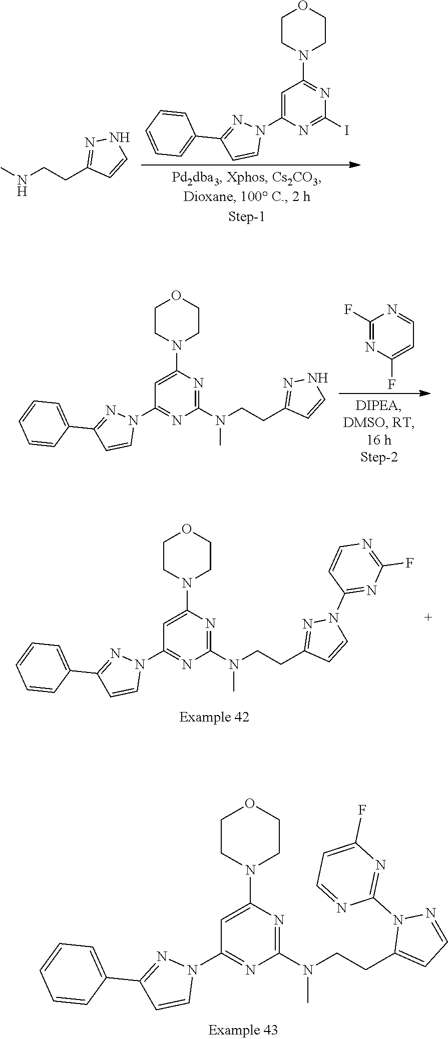

### Step 1: Synthesis of N-(2-(1H-pyrazol-3-yl)ethyl)-N-methyl-4-morpholino-6-(3-phenyl-1H-pyrazol-1-yl)pyrimidin-2-amine

To a stirred solution of 4-(2-iodo-6-(3-phenyl-1H-pyrazol-1-yl)pyrimidin-4-yl)morpholine (WO2021163727 A1)(0.3 g, 0.693 mmol) and N-methyl-2-(1H-pyrazol-3-yl) ethan-1-amine (0.130 g, 1.039 mmol) in dioxane (3 mL) was added Cs₂CO₃ (0.903 g, 2.770 mmol) at room temperature under nitrogen atmosphere. The reaction mixture was purged with nitrogen gas for 10 min. Pd₂(dba)₃ (0.095 g, 0.104 mmol) and XPhos (CAS: 564483-18-7)(0.165 g, 0.346 mmol) were added and the reaction mixture was heated at 100° C. for 2 h. After completion, the reaction mixture was diluted with water (50 mL) and the aqueous layer was extracted with EtOAc (2×100 mL). The combined organic phases were dried over sodium sulfate and evaporated under reduced pressure. The crude residue was purified by silica gel flash chromatography and pure compound was eluted at 1.5% MeOH in DCM to obtain title compound as an off-white solid (0.18 g, 0.418 mmol, 40%).

LCMS(Method-H): Retention time: 3.612 min, ES(+ve): 431.6 [M+H]⁺

### Step 2: Synthesis of N-(2-(1-(2-fluoropyrimidin-4-yl)-1H-pyrazol-3-yl)ethyl)-N-methyl-4-morpholino-6-(3-phenyl-1H-pyrazol-1-yl)pyrimidin-2-amine and N-(2-(1-(4-fluoropyrimidin-2-yl)-1H-pyrazol-5-yl)ethyl)-N-methyl-4-morpholino-6-(3-phenyl-1H-pyrazol-1-yl)pyrimidin-2-amine

To a stirred solution of N-(2-(1H-pyrazol-3-yl)ethyl)-N-methyl-4-morpholino-6-(3-phenyl-1H-pyrazol-1-yl)pyrimidin-2-amine (0.180 g, 0.418 mmol) in DMSO (1.8 mL), was added DIPEA (0.29 mL, 1.672 mmol) and 2,4-difluoropyrimidine (0.097 g, 0.836 mmol) at room temperature under nitrogen atmosphere. The reaction mixture was stirred at room temperature for 16 h. After completion, the reaction mixture was quenched by water (20 mL) and extracted with EtOAc (2×50 mL). The combined organic layer was dried over anhydrous sodium sulfate, filtered and solvent was removed in vacuum to obtain crude product. The resulting crude product was purified by column chromatography. Elution with 10% EtOAc in hexanes afforded N-(2-(1-(2-fluoropyrimidin-4-yl)-1H-pyrazol-3-yl)ethyl)-N-methyl-4-morpholino-6-(3-phenyl-1H-pyrazol-1-yl)pyrimidin-2-amine (Example 42) as an off-white solid (0.023 g, 0.044 mmol, 10%).

¹H NMR (400 MHz, DMSO-d6): δ 3.01 (t, 2H), 3.15 (s, 3H), 3.58-3.71 (m, 8H), 3.90-4.00 (m, 2H), 6.54 (s, 1H), 6.62 (s, 1H), 7.01 (s, 1H), 7.36-7.40 (m, 1H), 7.44-7.48 (m, 2H), 7.79 (s, 1H), 7.95 (d, 2H), 8.54 (s, 1H), 8.60 (s, 1H), 8.75-8.81 (m, 1H).

¹⁹F NMR (400 MHz, DMSO-d6): δ−45.56 (1F).

LCMS(Method-C Fast): Retention time: 2.312 min, ES(+ve): 527.8 [M+H]⁺

Further elution with 40% EtOAc in hexane afforded N-(2-(1-(4-fluoropyrimidin-2-yl)-1H-pyrazol-5-yl)ethyl)-N-methyl-4-morpholino-6-(3-phenyl-1H-pyrazol-1-yl)pyrimidin-2-amine (Example 43) as an off-white solid (0.014 g, 0.026 mmol, 6%).

¹H NMR (400 MHz, DMSO-d6): δ 2.91-3.07 (m, 3H), 3.42-3.74 (m, 10H), 3.86-3.97 (m, 2H), 6.38-6.57 (m, 1H), 6.51 (s, 1H), 7.00 (s, 1H), 7.28-7.33 (m, 1H), 7.36-7.40 (m, 1H), 7.44-7.48 (m, 2H), 7.71 (s, 1H), 7.96 (d, 2H), 8.27-8.63 (br. m, 1H), 8.86-8.97 (m, 1H).

¹⁹F NMR (400 MHz, DMSO-d6): δ−58.38 (1F).

LCMS(Method-C): Retention time: 2.109 min, ES(+ve): 527.5 [M+H]⁺

### Example 44: N-(2-(1-(4-fluoropyrimidin-2-yl)-1H-pyrazol-4-yl)ethyl)-4-morpholino-6-(1H-pyrazol-1-yl)pyrimidin-2-amine

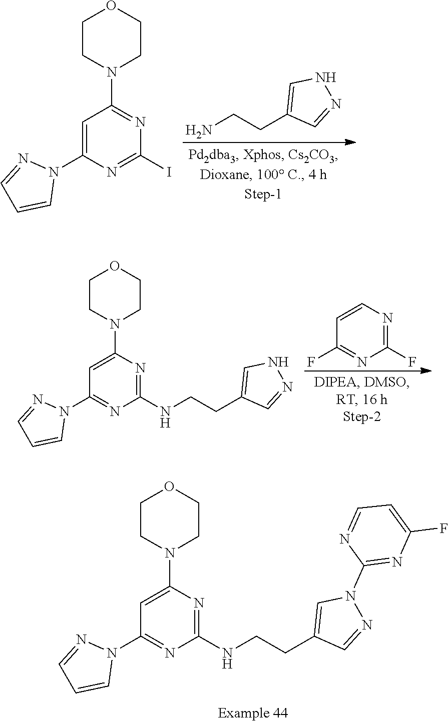

### Step 1: Synthesis of N-(2-(1H-pyrazol-4-yl)ethyl)-4-morpholino-6-(1H-pyrazol-1-yl)pyrimidin-2-amine

Prepared by the method of Example 26, Step 3 using 2-(1H-pyrazol-4-yl) ethan-1-amine (0.155 g, 1.399 mmol). The resulting crude product was purified by silica gel flash chromatography eluted with 2-4% MeOH in DCM to obtain title compound as a yellow solid (0.10 g, 0.293 mmol, 42%).

LCMS(Method-C): Retention time: 1.409 min, ES(+ve): 341.4 [M+H]⁺

### Step 2: Synthesis of N-(2-(1-(4-fluoropyrimidin-2-yl)-1H-pyrazol-4-yl)ethyl)-4-morpholino-6-(1H-pyrazol-1-yl)pyrimidin-2-amine

Prepared by the method of Example 28 and Example 29 using N-(2-(1H-pyrazol-4-yl)ethyl)-4-morpholino-6-(1H-pyrazol-1-yl)pyrimidin-2-amine (0.10 g, 0.293 mmol). The crude product was purified by column chromatography eluted with 70% EtOAc in hexanes to obtain the title product as off white solid (0.020 g, 0.045 mmol, 15%).

¹H NMR (400 MHz, DMSO-d6): δ 2.77-2.83 (m, 2H), 3.51-3.61 (m, 6H), 3.63-3.69 (m, 4H), 6.45 (s, 1H), 6.51-6.52 (m, 1H), 6.97-7.11 (m, 1H), 7.29-7.31 (m, 1H), 7.77 (d, 1H), 7.82 (s, 1H) 8.37-8.63 (br. s, 1H), 8.51 (s, 1H), 8.90-8.94 (m, 1H).

¹⁹F NMR (400 MHz, DMSO-d6): δ−58.63 (1F).

LCMS(Method-J): Retention time: 3.155 min, ES(+ve): 437.0 [M+H]⁺

### Example 45: N-(2-(1-(2-fluoropyrimidin-4-yl)-1H-pyrazol-4-yl)ethyl)-N-methyl-4-(3-methyl-1H-pyrazol-1-yl)-6-morpholinopyrimidin-2-amine

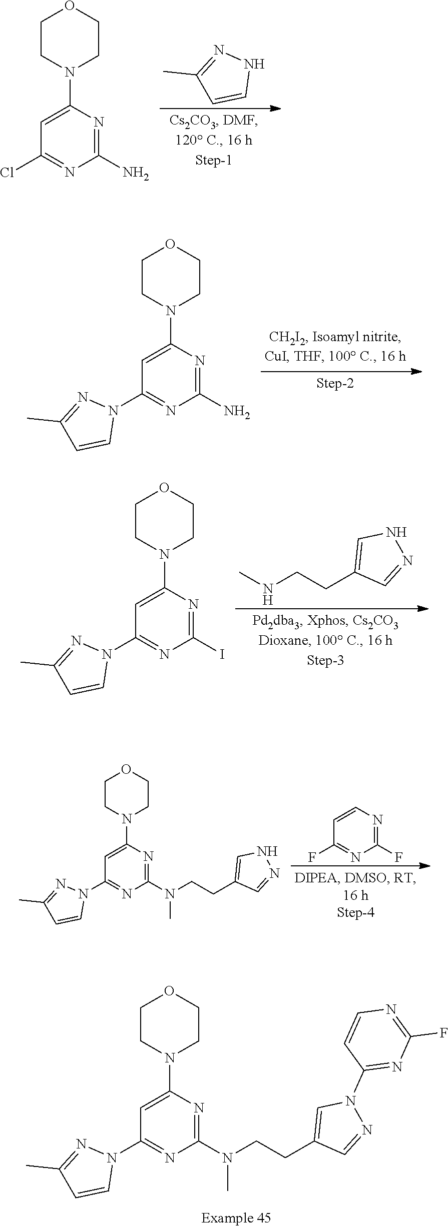

### Step 1: Synthesis of 4-(3-methyl-1H-pyrazol-1-yl)-6-morpholinopyrimidin-2-amine

Prepared by a method analogous to Example 26, Step 1 using 3-methyl-1H-pyrazole (1.0 g, 12.178 mmol). The reaction mixture was heated at 120° C. for 16 h. The crude product was purified by silica gel flash chromatography. The title product was eluted with 40-50% EtOAc in hexane to obtain a white solid (1.90 g, 7.30 mmol, 54%).

LCMS(Method-C): Retention time: 1.377 min, ES(+ve): 261.0 [M+H]⁺

### Step 2: Synthesis of 4-(2-iodo-6-(3-methyl-1H-pyrazol-1-yl)pyrimidin-4-yl)morpholine

Prepared by a method analogous to Example 26, Step 2 using 4-(3-methyl-1H-pyrazol-1-yl)-6-morpholinopyrimidin-2-amine (1.90 g, 7.307 mmol). The reaction mixture was heated at 100° C. for 16 h. The title compound was obtained as a brown solid (0.450 g, 1.21 mmol, 16%).

LCMS(Method-C): Retention time: 2.043 min, ES(+ve): 372.2 [M+H]⁺

### Step 3: Synthesis of N-(2-(1H-pyrazol-4-yl)ethyl)-N-methyl-4-(3-methyl-1H-pyrazol-1-yl)-6-morpholinopyrimidin-2-amine

Prepared by a method analogous to Example 26, Step 3 using 4-(2-iodo-6-(3-methyl-1H-pyrazol-1-yl)pyrimidin-4-yl)morpholine (0.400 g, 0.538 mmol). The reaction mixture was heated at 100° C. for 16 h. The crude product was purified by silica gel flash chromatography. The title product was eluted with 70% EtOAc in hexane to obtain a brown solid (0.300 g, 0.815 mmol, 75%).

LCMS(Method-C): Retention time: 1.621 min, ES(+ve): 369.4 [M+H]⁺

### Step 4: Synthesis of N-(2-(1-(2-fluoropyrimidin-4-yl)-1H-pyrazol-4-yl)ethyl)-N-methyl-4-(3-methyl-1H-pyrazol-1-yl)-6-morpholinopyrimidin-2-amine

Prepared by the method of Example 28 and Example 29 using N-(2-(1H-pyrazol-4-yl)ethyl)-N-methyl-4-(3-methyl-1H-pyrazol-1-yl)-6-morpholinopyrimidin-2-amine (0.300 g, 0.814 mmol). The crude product was purified by silica gel flash chromatography. The title product was eluted with 40-70% EtOAc in hexane to obtain an off-white solid (0.012 g, 0.025 mmol, 3%).

¹H NMR (400 MHz, DMSO-d6): δ 2.26 (s, 3H), 2.83 (t, 2H), 3.11 (s, 3H), 3.52-3.59 (m, 4H), 3.64-3.70 (m, 4H), 3.83-3.90 (m, 2H), 6.31-6.34 (m, 2H), 7.78-7.83 (m, 1H), 7.91 (s, 1H), 8.43 (d, 1H), 8.53 (s, 1H), 8.75-8.79 (m, 1H).

¹⁹F NMR (400 MHz, DMSO-d6): δ−45.65 (1F).

LCMS(Method-C): Retention time: 1.929 min, ES(+ve): 465.4 [M+H]⁺.

### Example 46:4-(3-chloro-1H-pyrazol-1-yl)-N-(2-(1-(2-fluoropyrimidin-4-yl)-1H-pyrazol-4-yl)ethyl)-N-methyl-6-morpholinopyrimidin-2-amine

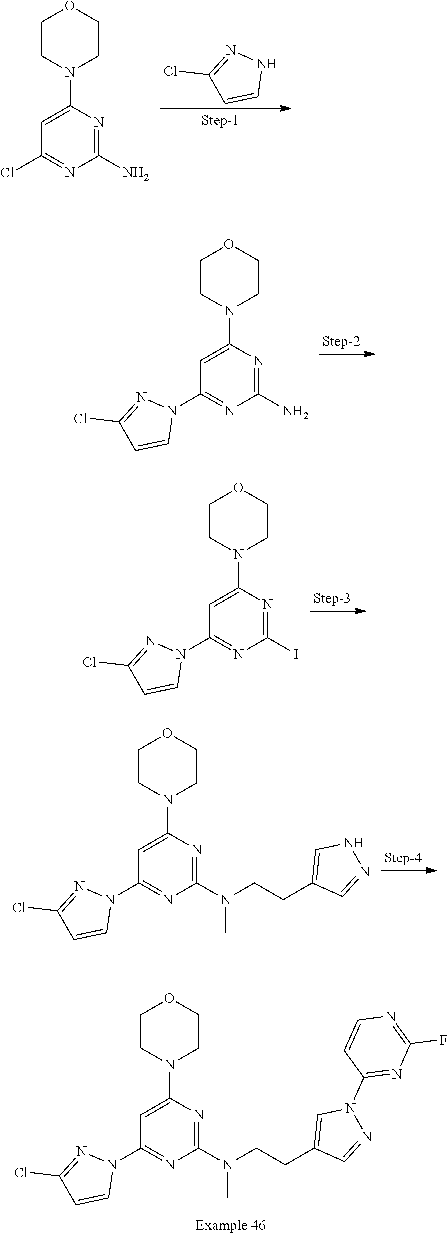

Prepared by a synthetic sequence directly analogues to Example 45 using 3-chloro-1H-pyrazole (2.3 g, 10.731 mmol) in Step 1.

### Step 1:4-(3-chloro-1H-pyrazol-1-yl)-6-morpholinopyrimidin-2-amine Yield (2.9 g, crude). Off-white solid

LCMS(Method-C): Retention time: 1.528 min, ES(+ve): 281.2/283.2 [M+H]

### Step 2:4-(6-(3-chloro-1H-pyrazol-1-yl)-2-iodopyrimidin-4-yl)morpholine Yield (0.8 g, 2.042 mmol, 38%). Brown solid

LCMS(Method-C): Retention time: 2.161 min, ES(+ve): 392.2/394.2 [M+H]⁺

### Step 3: N-(2-(1H-pyrazol-4-yl)ethyl)-4-(3-chloro-1H-pyrazol-1-yl)-N-methyl-6-morpholinopyrimidin-2-amine Yield (0.140 g, 0.360 mmol, 33%). Brown oil

¹H NMR (400 MHz, DMSO-d6): δ 2.65-2.75 (m, 2H), 3.09 (s, 3H), 3.53-3.82 (m, 10H), 6.34 (s, 1H), 6.64 (s, 1H), 7.28-7.63 (m, 2H), 8.59 (s, 1H), 12.53 (s, 1H).

LCMS(Method-C): Retention time: 1.756 min, ES(+ve): 389.4/391.3 [M+H]⁺

### Step 4:4-(3-chloro-1H-pyrazol-1-yl)-N-(2-(1-(2-fluoropyrimidin-4-yl)-1H-pyrazol-4-yl)ethyl)-N-methyl-6-morpholinopyrimidin-2-amine

Yield (0.060 g, 1.237 mmol, 34%). White solid.

¹H NMR (400 MHz, DMSO-d6): δ 2.82 (t, 2H), 3.10 (s, 3H), 3.53-3.60 (m, 4H), 3.61-3.67 (m, 4H), 3.82-3.90 (m, 2H), 6.30 (s, 1H), 6.62 (d, 1H), 7.76-7.81 (m, 1H), 7.90 (s, 1H), 8.52 (s, 1H), 8.61 (d, 1H), 8.76 (d, 1H).

¹⁹F NMR (400 MHz, DMSO-d6): δ−45.64 (1F).

LCMS(Method-C): Retention time: 2.114 min, ES(+ve): 485.4/487.4 [M+H]⁺

### Example 47:4-(3-bromo-1H-pyrazol-1-yl)-N-(2-(1-(4-fluoropyrimidin-2-yl)-1H-pyrazol-4-yl)ethyl)-N-methyl-6-morpholinopyrimidin-2-amine

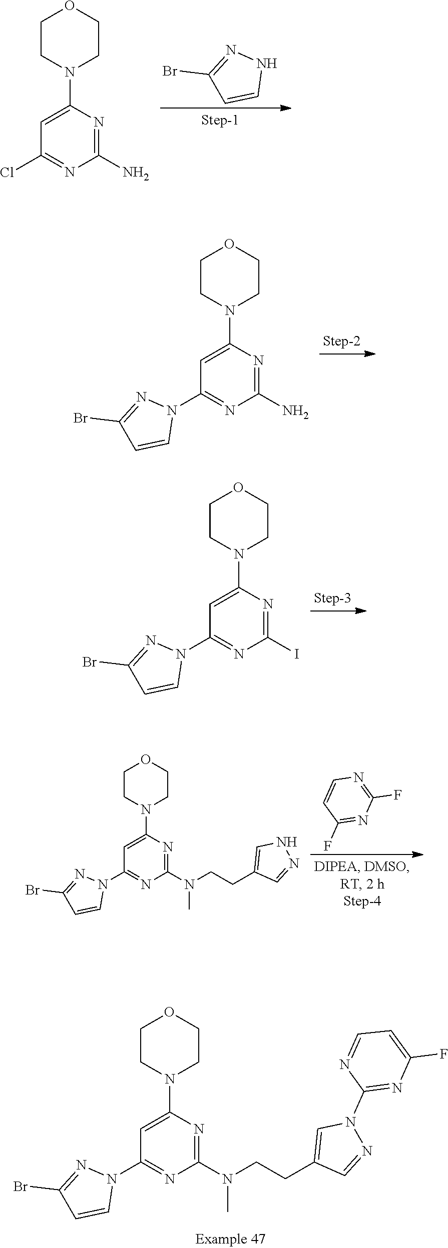

Prepared by a synthetic sequence very similar to Example 45 using 3-bromo-1H-pyrazole (1.80 g, 12.814 mmol) in Step 1, and stirring Step 4 for only 2 h to give the alternative fluoropyrimidine regio-isomer.

### Step 1:4-(3-bromo-1H-pyrazol-1-yl)-6-morpholinopyrimidin-2-amine Yield (3.5 g, 11.692 mmol, 92%). Off-white solid

¹H NMR (400 MHz, DMSO-d6): δ 3.53-3.69 (m, 8H), 6.36 (s, 1H), 6.45 (s, 2H), 6.67 (d, 1H), 8.37 (d, 1H).

### Step 2:4-(6-(3-bromo-1H-pyrazol-1-yl)-2-iodopyrimidin-4-yl)morpholine Yield (1.8 g, 4.128 mmol, 38%). Brown solid

LCMS(Method-J): Retention time: 3.665 min, ES(+ve): 436.1/438.1 [M+H]⁺

### Step 3: N-(2-(1H-pyrazol-4-yl)ethyl)-4-(3-bromo-1H-pyrazol-1-yl)-N-methyl-6-morpholinopyrimidin-2-amine Yield (0.110 g, 0.253 mmol, 36%). Brown solid

LCMS(Method-H): Retention time: 3.311 min, ES(+ve): 433.4/435.4 [M+H]⁺

### Step 4:4-(3-bromo-1H-pyrazol-1-yl)-N-(2-(1-(4-fluoropyrimidin-2-yl)-1H-pyrazol-4-yl)ethyl)-N-methyl-6-morpholinopyrimidin-2-amine (0.025 g, 0.047 mmol, 20%). White solid

¹H NMR (400 MHz, DMSO-d6): δ 2.81 (t, 2H), 3.10 (s, 3H), 3.53-3.71 (m, 8H), 3.80-3.90 (m, 2H), 6.32 (s, 1H), 6.68 (t, 1H), 7.28 (d, 1H), 7.80 (s, 1H), 8.49 (s, 1H), 8.56 (t, 1H), 8.87-8.91 (m, 1H).

¹⁹F NMR (400 MHz, DMSO-d6): δ−53.887 (1F).

LCMS(Method-H): Retention time: 3.697 min, ES(+ve): 529.4/531.4 [M+H]⁺

### Example 48: N-(2-(1-(2-fluoropyrimidin-4-yl)-1H-pyrazol-3-yl)ethyl)-N-methyl-4-morpholino-6-(1H-pyrazol-1-yl)pyrimidin-2-amine

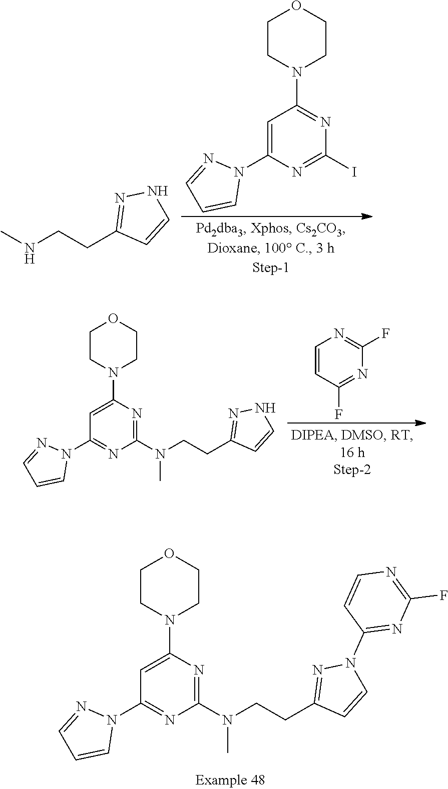

### Step 1: Synthesis of N-(2-(1H-pyrazol-3-yl)ethyl)-N-methyl-4-morpholino-6-(1H-pyrazol-1-yl)pyrimidin-2-amine

4-(2-Iodo-6-(1H-pyrazol-1-yl)pyrimidin-4-yl)morpholine (Examples 26 and 27, Step 2)(0.3 g, 0.839 mmol) and N-methyl-2-(1H-pyrazol-3-yl) ethan-1-amine (0.157 g, 1.259 mmol) were added to dioxane (3 mL). To the above solution, Cs₂CO₃ (1.094 g, 3.359 mmol) was added at room temperature under nitrogen atmosphere. The reaction mixture was purged with nitrogen gas for 10 min. Pd₂(dba)₃ (0.115 g, 0.130 mmol) and XPhos (CAS: 564483-18-7)(0.200 g, 0.419 mmol) were added, and the reaction mixture was heated at 100° C. for 3 h. After completion, the reaction mixture was diluted with water (50 mL) and the aqueous layer was extracted with EtOAc (2×100 mL). The combined organic phases were dried over sodium sulfate and evaporated under reduced pressure. The crude residue was purified by silica gel flash chromatography and pure compound was eluted at 3% MeOH in DCM to obtain title compound as an off white solid (0.160 g, 0.451 mmol, 36%).

LCMS(Method-H): Retention time: 2.832 min, ES(+ve): 355.5 [M+H]⁺

### Step 2: N-(2-(1-(2-fluoropyrimidin-4-yl)-1H-pyrazol-3-yl)ethyl)-N-methyl-4-morpholino-6-(1H-pyrazol-1-yl)pyrimidin-2-amine

To a stirred solution of N-(2-(1H-pyrazol-3-yl)ethyl)-N-methyl-4-morpholino-6-(1H-pyrazol-1-yl)pyrimidin-2-amine (0.15 g, 0.423 mmol) in DMSO (1.5 mL), was added DIPEA (0.29 mL, 1.693 mmol) and 2,4-difluoropyrimidine (0.098 g, 0.846 mmol) at room temperature under nitrogen atmosphere. The reaction mixture was stirred at room temperature for 16 h. After completion, the reaction mixture was quenched with water and extracted with EtOAc (2×50 mL). The combined organic layer was dried over anhydrous sodium sulfate, filtered and concentrated under reduced pressure to obtain crude. The resulting crude was purified by column chromatography. The product was eluted in 30% EtOAc in hexane to obtain the title compound (0.024 g, 0.053 mmol, 13%) as a white solid.

¹H NMR (400 MHz, DMSO-d6): δ 2.99 (t, 2H), 3.13 (s, 3H), 3.53-3.70 (m, 8H), 3.88-3.97 (m, 2H), 6.43 (s, 1H), 6.49 (s, 1H), 6.61 (s, 1H), 7.73-7.82 (m, 2H), 8.53 (s, 2H), 8.73-8.80 (m, 1H).

¹⁹F NMR (400 MHz, DMSO-d6): δ−45.57 (1F).

LCMS(Method-C): Retention time: 1.946 min, ES(+ve): 451.4 [M+H]⁺

Characterizing analytical data for compounds shown in Table 1

| | ¹H NMR | ¹⁹F NMR | LCMS |
|---|---|---|---|
| Example 49 | ¹H NMR (400 MHz, DMSO-d₆): δ 1.17 (t, 3H), 2.87 | ¹⁹F NMR (400 MHz, | LCMS (Method-H): |
| | (t, 2H), 3.56-3.74 (m, 10H), 3.77-3.90 (m, 2H), | DMSO-d6): δ −45.622 | ES(+ve): 541.0 |
| | 6.56 (s, 1H), 7.03 (d, 1H), 7.37-7.43 (m, 1H), 7.47 | (1F). | [M + H]⁺. |
| | (t, 2H), 7.81-7.85 (m, 1H), 7.90-8.06 (m, 3H), 8.52- | | |
| | 8.63 (m, 2H), 8.78 (d, 1H). | | |
| Example 50 | ¹H NMR (400 MHz, DMSO-d₆): δ 2.81 (t, 2H), | ¹⁹F NMR (400 MHz, | LCMS (Method-C): |
| | 3.52-3.74 (m, 10H), 6.46 (s, 1H), 6.52 (t, 1H), 7.05 | DMSO-d6): δ −45.631 | ES(+ve): 437.4 |
| | (br. s, 1H), 7.77 (s, 1H), 7.82-7.86 (m, 1H), 7.93 (s, | (1F). | [M + H]⁺. |
| | 1H), 8.43 (br. s, 1H), 8.54 (s, 1H), 8.77-8.82 (m, 1H). | | |
| Example 51 | ¹H NMR (400 MHz, DMSO-d₆): δ 1.15 (t, 3H), 2.85 | ¹⁹F NMR (400 MHz, | LCMS (Method-H): |
| | (t, 2H), 3.56-3.74 (m, 10H), 3.75-3.90 (m, 2H), | DMSO-d6): δ −58.622 | ES(+ve): 541.0 |
| | 6.56 (s, 1H), 7.03 (d, 1H), 7.26-7.32 (m, 1H), 7.35- | (1F). | [M + H]⁺. |
| | 7.42 (m, 1H), 7.46 (t, 2H), 7.85 (br. s, 1H), 7.96 (d, | | |
| | 2H), 8.53 (br. s, 1H), 8.58 (s, 1H), 8.88-8.97 (m, 1H). | | |
| Example 52 | ¹H NMR (400 MHz, DMSO-d₆): δ 1.14 (t, 3H), 2.83 | ¹⁹F NMR (400 MHz, | LCMS (Method-H): |
| | (t, 2H), 3.53-3.72 (m, 10H), 3.73-3.87 (m, 2H), | DMSO-d6): δ −58.636 | ES(+ve): 465.0 |
| | 6.45 (s, 1H), 6.51 (s, 1H), 7.29 (d, 1H), 7.77 (s, 1H), | (1F). | [M + H]⁺. |
| | 7.82 (br. s, 1H), 8.53 (s, 2H), 8.87-8.97 (m, 1H). | | |
| Example 53 | ¹H NMR (400 MHz, DMSO-d₆): δ 1.14 (t, 3H), 2.84 | ¹⁹F NMR (400 MHz, | LCMS (Method-H): |
| | (t, 2H), 3.53-3.70 (m, 10H), 3.73-3.85 (m, 2H), | DMSO-d6): δ −45.638 | ES(+ve): 465.0 |
| | 6.45 (s, 1H), 6.51 (s, 1H), 7.76 (s, 1H), 7.78-7.83 | (1F). | [M + H]⁺. |
| | (m, 1H), 7.88-7.98 (m, 1H), 8.50-8.60 (m, 2H), | | |
| | 8.74-8.81 (m, 1H). | | |
| Example 54 | ¹H NMR (400 MHz, DMSO-d₆): δ 0.69-0.75 (m, | ¹⁹F NMR (400 MHz, | LCMS (Method-J): |
| | 2H), 0.89-0.96 (m, 2H), 1.92-2.00 (m, 1H), 2.82 (t, | DMSO-d6): δ −45.627 | ES(+ve): 491.5 |
| | 2H), 3.10 (s, 3H), 3.51-3.72 (m, 8H), 3.81-3.89 (m, | (1F). | [M + H]⁺. |
| | 2H), 6.17-6.22 (m, 1H), 6.31 (s, 1H), 7.77-7.82 (m, | | |
| | 1H), 7.90 (s, 1H), 8.37-8.42 (m, 1H), 8.51 (s, 1H), | | |
| | 8.73-8.78 (m, 1H). | | |
| Example 55 | ¹H NMR (400 MHz, DMSO-d₆): δ 0.70-0.76 (m, | ¹⁹F NMR (400 MHz, | LCMS (Method-J): |
| | 2H), 0.89-0.96 (m, 2H), 1.92-2.00 (m, 1H), 2.81 (t, | DMSO-d6): δ −58.473 | ES(+ve): 491.4 |
| | 2H), 3.10 (s, 3H), 3.52-3.60 (m, 4H), 3.62-3.70 (m, | (1F). | [M + H]⁺. |
| | 4H), 3.80-3.88 (m, 2H), 6.17-6.22 (m, 1H), 6.32 (s, | | |
| | 1H), 7.25-7.32 (m, 1H), 7.79 (s, 1H), 8.37-8.42 (m, | | |
| | 1H), 8.49 (s, 1H), 8.87-8.94 (m, 1H). | | |
| Example 56 | ¹H NMR (400 MHz, DMSO-d₆): δ 2.82 (t, 2H), 3.11 | ¹⁹F NMR (400 MHz, | LCMS (Method-C): |
| | (s, 3H), 3.53-3.70 (m, 8H), 3.82-3.92 (m, 2H), 6.32 | DMSO-d6): δ −45.646 | ES(+ve): 529.4/531.3 |
| | (s, 1H), 6.65-6.70 (m, 1H), 7.76-7.83 (m, 1H), 7.90 | (1F). | [M + H]⁺. |
| | (s, 1H), 8.50-8.55 (m, 1H), 8.57 (d, 1H), 8.73-8.78 | | |
| | (m, 1H). | | |
| Example 57 | ¹H NMR (400 MHz, DMSO-d₆): δ 2.85 (t, 2H), 3.13 | ¹⁹F NMR (400 MHz, | LCMS (Method- |
| | (s, 3H), 3.52-3.64 (m, 4H), 3.66-3.76 (m, 4H), 3.81- | DMSO-d6): δ −45.605 | S_FAST): ES(+ve): |
| | 3.88 (m, 2H), 7.00-7.04 (m, 1H), 7.35-7.40 (m, | (1F). | 561.0/562.9 [M + H]⁺. |
| | 1H), 7.45 (t, 2H), 7.77-7.83 (m, 1H), 7.87-7.97 (m, | | |
| | 3H), 8.33-8.40 (m, 1H), 8.51 (s, 1H), 8.74-8.79 (m, 1H). | | |
| Example 58 | ¹H NMR (400 MHz, DMSO-d₆): δ 2.85 (t, 2H), 3.14 | ¹⁹F NMR (400 MHz, | LCMS (Method- |
| | (s, 3H), 3.53-3.63 (m, 4H), 3.68-3.77 (m, 4H), 3.84 | DMSO-d6): δ −58.595 | S_FAST): ES(+ve): |
| | (t, 2H), 7.02 (d, 1H), 7.27-7.31 (m, 1H), 7.35-7.42 | (1F). | 560.9/563.0 [M + H]⁺. |
| | (m, 1H), 7.46 (t, 2H), 7.79-7.85 (m, 1H), 7.90 (d, | | |
| | 2H), 8.33-8.40 (m, 1H), 8.49 (s, 1H), 8.88-8.94 (m, 1H). | | |
| Example 59 | ¹H NMR (400 MHz, DMSO-d₆): δ 2.86 (t, 2H), 3.15 | ¹⁹F NMR (400 MHz, | LCMS (Method-J): |
| | (s, 3H), 3.57-3.62 (m, 4H), 3.67-3.72 (m, 4H), 3.87- | DMSO-d6): δ −45.627 | ES(+ve): 527.3 |
| | 3.94 (m, 2H), 6.67 (s, 1H), 7.05 (d, 1H), 7.36 (t, | (1F). | [M + H]⁺. |
| | 1H), 7.54 (t, 2H), 7.80-7.83 (m, 1H), 7.93-7.97 (m, | | |
| | 3H), 8.53 (s, 1H), 8.56 (d, 1H), 8.76-8.78 (m, 1H). | | |
| Example 60 | ¹H NMR (400 MHz, DMSO-d₆): δ 2.85 (t, 2H), 3.15 | ¹⁹F NMR (400 MHz, | LCMS (Method-C): |
| | (s, 3H), 3.57-3.62 (m, 4H), 3.67-3.72 (m, 4H), 3.85- | DMSO-d6): δ −58.636 | ES(+ve): 527.4 |
| | 3.94 (m, 2H), 6.68 (s, 1H), 7.05 (d, 1H), 7.27-7.31 | (1F). | [M + H]⁺. |
| | (m, 1H), 7.35 (t, 1H), 7.54 (t, 2H), 7.83 (s, 1H), | | |
| | 7.95 (d, 2H), 8.51 (s, 1H), 8.57 (d, 1H), 8.89-8.95 | | |
| | (m, 1H). | | |
| Example 61 | ¹H NMR (400 MHz, DMSO-d₆): δ 1.12-1.30 (m, | ¹⁹F NMR (400 MHz, | LCMS (Method-H): |
| | 3H), 2.80-2.92 (m, 2H), 3.07-3.23 (m, 4H) 3.40- | DMSO-d6): δ −45.631 | ES(+ve): 541.4 |
| | 3.51 (m, 1H), 3.58-3.65 (m, 1H), 3.70-3.77 (m, | (1F). | [M + H]⁺. |
| | 1H), 3.80-4.08 (m, 4H), 4.37-4.47 (m, 1H), 6.50 (s, | | |
| | 1H), 7.03 (s, 1H), 7.36-7.43 (m, 1H), 7.44-7.52 (m, | | |
| | 2H), 7.77-7.85 (m, 1H), 7.89-8.03 (m, 3H), 8.50- | | |
| | 8.56 (m, 1H), 8.60-8.66 (m, 1H), 8.73-8.82 (m, 1H). | | |
| Example 62 | ¹H NMR (400 MHz, DMSO-d₆): δ 1.16-1.23 (m, | ¹⁹F NMR (400 MHz, | LCMS (Method-H): |
| | 3H), 2.80-2.89 (m, 2H), 3.08-3.22 (m, 4H) 3.42- | DMSO-d6): δ −58.640 | ES(+ve): 541.2 |
| | 3.51 (m, 1H), 3.58-3.64 (m, 1H), 3.70-3.76 (m, | (1F). | [M + H]⁺. |
| | 1H), 3.80-4.06 (m, 4H), 4.39-4.47 (m, 1H), 6.50 (s, | | |
| | 1H), 7.01-7.05 (m, 1H), 7.26-7.32 (m, 1H), 7.36- | | |
| | 7.42 (m, 1H), 7.43-7.50 (m, 2H), 7.78-7.83 (m, | | |
| | 1H), 7.93-8.00 (m, 2H), 8.47-8.57 (m, 1H), 8.60- | | |
| | 8.66 (m, 1H), 8.87-8.95 (m, 1H). | | |
| Example 63 | ¹H NMR (400 MHz, DMSO-d₆): δ 2.17 (s, 3H), 2.84 | ¹⁹F NMR (400 MHz, | LCMS (Method-C): |
| | (t, 2H), 3.12 (s, 3H), 3.66-3.75 (m, 4H), 3.85 (t, | DMSO-d6): δ −45.619 | ES(+ve): 541.4 |
| | 2H), 6.97-7.02 (m, 1H), 7.33-7.40 (m, 1H), 7.45 (t, | (1F). | [M + H]⁺. |
| | 2H), 7.78-7.82 (m, 1H), 7.88-7.95 (m, 3H), 8.43- | | |
| | 8.52 (m, 2H), 8.72-8.78 (m, 1H). Four protons | | |
| | obscured by water peak. | | |
| Example 64 | ¹H NMR (400 MHz, DMSO-d₆): δ 2.18 (s, 3H), 2.84 | ¹⁹F NMR (400 MHz, | LCMS (Method-C): |
| | (t, 2H), 3.13 (s, 3H), 3.68-3.73 (m, 4H), 3.84 (t, | DMSO-d6): δ −58.631 | ES(+ve): 541.4 |
| | 2H), 6.98-7.02 (m, 1H), 7.26-7.30 (m, 1H), 7.33- | (1F). | [M + H]⁺. |
| | 7.40 (m, 1H), 7.45 (t, 2H), 7.78-7.82 (m, 1H), 7.91 | | |
| | (d, 2H), 8.44-8.50 (m, 2H), 8.87-8.94 (m, 1H). Four | | |
| | protons obscured by water peak. | | |
| Example 65 | ¹H NMR (400 MHz, DMSO-d₆): δ 2.84 (t, 2H), 3.10 | ¹⁹F NMR (400 MHz, | LCMS (Method-C): |
| | (s, 3H), 3.65-3.74 (m, 8H), 3.82 (t, 2H), 7.06 (d, | DMSO-d6): δ −45.621 | ES(+ve): 545.6 |
| | 1H), 7.36-7.42 (m, 1H), 7.46 (t, 2H), 7.80-7.84 (m, | (1F), −168.177 (1F). | [M + H]⁺. |
| | 1H), 7.88-7.94 (m, 3H), 8.50 (d, 1H), 8.52 (s, 1H), | | |
| | 8.76-8.78 (m, 1H). | | |
| Example 66 | ¹H NMR (400 MHz, DMSO-d₆): δ 2.84 (t, 2H), 3.11 | ¹⁹F NMR (400 MHz, | LCMS (Method-C): |
| | (s, 3H), 3.65-3.74 (m, 8H), 3.82 (t, 2H), 7.06 (d, | DMSO-d6): δ −58.615 | ES(+ve): 545.3 |
| | 1H), 7.27-7.31 (m, 1H), 7.36-7.42 (m, 1H), 7.47 (t, | (1F), −168.153 (1F). | [M + H]⁺. |
| | 2H), 7.82 (s, 1H), 7.89-7.94 (m, 2H), 8.48-8.53 (m, | | |
| | 2H), 8.88-8.95 (m, 1H). | | |
| Example 67 | ¹H NMR (400 MHz, DMSO-d₆): δ 1.20 (d, 3H), 2.86 | ¹⁹F NMR (400 MHz, | LCMS (Method-C): |
| | (t, 2H), 3.08-3.22 (m, 4H) 3.42-3.51 (m, 1H), 3.58- | DMSO-d6): δ −45.633 | ES(+ve): 541.4 |
| | 3.63 (m, 1H), 3.72-3.77 (m, 1H), 3.80-4.06 (m, | (1F). | [M + H]⁺. |
| | 4H), 4.38-4.46 (m, 1H), 6.50 (s, 1H), 7.02 (d, 1H), | | |
| | 7.37-7.42 (m, 1H), 7.44-7.50 (m, 2H), 7.77-7.83 | | |
| | (m, 1H), 7.90-7.98 (m, 3H), 8.52-8.55 (m, 1H), | | |
| | 8.63 (d, 1H), 8.75-8.79 (m, 1H). | | |
| Example 68 | ¹H NMR (400 MHz, DMSO-d₆): δ 1.20 (d, 3H), 2.85 | ¹⁹F NMR (400 MHz, | LCMS (Method-C): |
| | (t, 2H), 3.08-3.23 (m, 4H) 3.42-3.52 (m, 1H), 3.59- | DMSO-d6): δ −58.640 | ES(+ve): 541.4 |
| | 3.65 (m, 1H), 3.72-3.77 (m, 1H), 3.79-4.07 (m, | (1F). | [M + H]⁺. |
| | 4H), 4.38-4.47 (m, 1H), 6.51 (s, 1H), 7.03 (d, 1H), | | |
| | 7.27-7.31 (m, 1H), 7.37-7.43 (m, 1H), 7.45-7.51 | | |
| | (m, 2H), 7.80-7.86 (m, 1H), 7.95-7.99 (m, 2H), | | |
| | 8.50-8.55 (m, 1H), 8.63 (d, 1H), 8.88-8.96 (m, 1H). | | |
| Example 69 | ¹H NMR (400 MHz, DMSO-d₆): δ 2.71 (s, 3H), 2.85 | ¹⁹F NMR (400 MHz, | LCMS (Method-H): |
| | (t, 2H), 3.13 (s, 3H), 3.57-3.73 (m, 8H), 3.84 (t, | DMSO-d6): δ −45.607 | ES(+ve): 541.4 |
| | 2H), 6.56 (s, 1H), 6.78 (s, 1H), 7.34-7.40 (m, 1H), | (1F). | [M + H]⁺. |
| | 7.42-7.47 (m, 2H), 7.82-7.85 (m, 1H), 7.87-7.93 | | |
| | (m, 3H), 8.52 (s, 1H), 8.77-8.80 (m, 1H). | | |
| Example 70 | ¹H NMR (400 MHz, DMSO-d₆): δ 2.72 (s, 3H), 2.84 | ¹⁹F NMR (400 MHz, | LCMS (Method-H): |
| | (t, 2H), 3.13 (s, 3H), 3.57-3.73 (m, 8H), 3.84 (t, | DMSO-d6): δ −58.602 | ES(+ve): 541.2 |
| | 2H), 6.57 (s, 1H), 6.78 (s, 1H), 7.28-7.32 (m, 1H), | (1F). | [M + H]⁺. |
| | 7.35-7.41 (m, 1H), 7.42-7.48 (m, 2H), 7.81 (s, 1H), | | |
| | 7.87-7.93 (m, 2H), 8.50 (s, 1H), 8.90-8.97 (m, 1H). | | |
| Example 71 | ¹H NMR (400 MHz, DMSO-d₆): δ 2.85 (t, 2H), 3.10 | ¹⁹F NMR (400 MHz, | LCMS (Method-X): |
| | (s, 3H), 3.63-3.75 (m, 8H), 3.80-3.92 (m, 2H), 6.47 | DMSO-d6): δ −45.596 | ES(+ve): 527.4 |
| | (s, 1H), 7.02 (d, 1H), 7.37-7.43 (m, 1H), 7.46 (t, | (1F). | [M + H]⁺. |
| | 2H), 7.78-7.83 (m, 1H), 7.90-7.98 (m, 3H), 8.53- | | |
| | 8.57 (m, 1H), 8.62 (d, 1H), 8.76-8.79 (m, 1H). | | |
| Example 72 | ¹H NMR (400 MHz, DMSO-d₆): δ 2.85 (t, 2H), 3.10 | ¹⁹F NMR (400 MHz, | LCMS (Method-X): |
| | (s, 3H), 3.64-3.75 (m, 8H), 3.80-3.92 (m, 2H), 6.47 | DMSO-d6): δ −58.621 | ES(+ve): 527.4 |
| | (s, 1H), 7.02 (d, 1H), 7.27-7.31 (m, 1H), 7.37-7.43 | (1F). | [M + H]⁺. |
| | (m, 1H), 7.47 (t, 2H), 7.81 (s, 1H), 7.94-7.98 (m, | | |
| | 2H), 8.51-8.55 (m, 1H), 8.63 (d, 1H), 8.87-8.94 (m, 1H). | | |
| Example 73 | ¹H NMR (400 MHz, DMSO-d₆): δ 2.88 (t, 2H), 3.13 | ¹⁹F NMR (400 MHz, | LCMS (Method-J): |
| | (s, 3H), 3.62-3.73 (m, 8H), 3.87-3.94 (m, 2H), 6.56 | DMSO-d6): δ −44.953 | ES(+ve): 527.5 |
| | (s, 1H), 7.04 (d, 1H), 7.37-7.43 (m, 1H), 7.47 (t, | (1F). | [M + H]⁺. |
| | 2H), 7.83-7.92 (m, 2H), 7.97 (d, 2H), 8.62-8.68 (m, | | |
| | 2H), 8.83-8.88 (m, 1H). | | |
| Example 74 | ¹H NMR (400 MHz, DMSO-d₆): δ 2.88 (t, 2H), 3.14 | ¹⁹F NMR (400 MHz, | LCMS (Method-J): |
| | (s, 3H), 3.58-3.75 (m, 8H), 3.87-3.95 (m, 2H), 6.56 | DMSO-d6): δ −58.530 | ES(+ve): 527.4 |
| | (s, 1H), 7.04 (d, 1H), 7.32-7.35 (m, 1H), 7.36-7.43 | (1F). | [M + H]⁺. |
| | (m, 1H), 7.47 (t, 2H), 7.73 (s, 1H), 7.97 (d, 2H), | | |
| | 8.50 (s, 1H), 8.64 (d, 1H), 8.91-8.97 (m, 1H). | | |
| Example 75 | ¹H NMR (400 MHz, DMSO-d₆): δ 3.00 (br. s, 3H), | ¹⁹F NMR (400 MHz, | LCMS (Method-J): |
| | 3.46-3.76 (m, 10H), 3.86 (br. s, 2H), 6.51 (s, 1H), | DMSO-d6): δ −44.638 | ES(+ve): 527.5 |
| | 7.01 (br. s, 2H), 7.37-7.43 (m, 1H), 7.47 (t, 2H), | (1F). | [M + H]⁺. |
| | 7.81 (br. s, 1H), 7.98 (d, 2H), 8.20-8.60 (br, m, 2H), | | |
| | 8.78-8.83 (m, 1H). | | |
| Example 76 | ¹H NMR (400 MHz, DMSO-d₆): δ 2.27 (s, 3H), 2.86 | ¹⁹F NMR (400 MHz, | LCMS (Method-H): |
| | (t, 2H), 3.14 (s, 3H), 3.55-3.72 (m, 8H), 3.84-3.94 | DMSO-d6): δ −45.633 | ES(+ve): 541.4 |
| | (m, 2H), 6.46 (s, 1H), 7.38-7.45 (m, 1H), 7.46-7.52 | (1F). | [M + H]⁺. |
| | (m, 2H), 7.75-7.83 (m, 3H), 7.92 (s, 1H), 8.43 (s, | | |
| | 1H), 8.54 (s, 1H), 8.75-8.79 (m, 1H). | | |
| Example 77 | ¹H NMR (400 MHz, DMSO-d₆): δ 2.28 (s, 3H), 2.85 | ¹⁹F NMR (400 MHz, | LCMS (Method-H): |
| | (t, 2H), 3.14 (s, 3H), 3.54-3.72 (m, 8H), 3.84-3.93 | DMSO-d6): δ −58.637 | ES(+ve): 541.4 |
| | (m, 2H), 6.47 (s, 1H), 7.27-7.32 (m, 1H), 7.38-7.45 | (1F). | [M + H]⁺. |
| | (m, 1H), 7.49 (t, 2H), 7.76-7.85 (m, 3H), 8.44 (s, | | |
| | 1H), 8.52 (s, 1H), 8.88-8.95 (m, 1H). | | |
| Example 78 | ¹H NMR (400 MHz, DMSO-d₆): δ 2.78-2.87 (m, | ¹⁹F NMR (400 MHz, | LCMS (Method-J): |
| | 2H), 3.08 (s, 3H), 3.53-3.72 (m, 8H), 3.82-3.92 (m, | DMSO-d6): δ −58.620 | ES(+ve): 527.3 |
| | 2H), 5.65 (s, 1H), 6.93-6.98 (m, 1H), 7.23-7.30 (m, | (1F). | [M + H]⁺. |
| | 1H), 7.31-7.39 (m, 1H), 7.40-7.47 (m, 2H), 7.87- | | |
| | 7.96 (m, 3H), 8.52-8.63 (m, 2H), 8.83-8.92 (m, 1H). | | |
| Example 79 | ¹H NMR (400 MHz, DMSO-d₆): δ 2.83 (t, 2H), 3.07 | ¹⁹F NMR (400 MHz, | LCMS (Method-J): |
| | (s, 3H), 3.60-3.93 (m, 10H), 6.40 (s, 1H), 7.26 (t, | DMSO-d6): δ −45.645 | ES(+ve): 527.2 |
| | 1H), 7.40 (t, 2H), 7.76 (d, 2H), 7.78-7.83 (m, 1H), | (1F). | [M + H]⁺. |
| | 7.90 (s, 1H), 8.25 (s, 1H), 8.55 (s, 1H), 8.77 (d, 1H), | | |
| | 8.99 (s, 1H). | | |
| Example 80 | ¹H NMR (400 MHz, DMSO-d₆): δ 3.01 (t, 2H), 3.15 | ¹⁹F NMR (400 MHz, | LCMS (Method-C): |
| | (s, 3H), 3.58-3.73 (m, 8H), 3.87-4.01 (m, 2H), 6.56 | DMSO-d6): δ −58.669 | ES(+ve): 527.3 |
| | (s, 2H), 6.97-7.04 (m, 1H), 7.28-7.32 (m, 1H), 7.37- | (1F). | [M + H]⁺. |
| | 7.42 (m, 1H), 7.48 (t, 2H), 7.97 (d, 2H), 8.54 (d, | | |
| | 1H), 8.64 (d, 1H), 8.90-8.97 (m, 1H). | | |
| Example 81 | ¹H NMR (400 MHz, DMSO-d₆): δ 2.83 (t, 2H), 3.08 | ¹⁹F NMR (400 MHz, | LCMS (Method-J): |
| | (s, 3H), 3.65-3.92 (m, 10H), 6.41 (s, 1H), 7.23-7.32 | DMSO-d6): δ −58.635 | ES(+ve): 527.3 |
| | (m, 2H), 7.41 (t, 2H), 7.75-7.82 (m, 3H), 8.23 (s, | (1F). | [M + H]⁺. |
| | 1H), 8.53 (s, 1H), 8.90-8.97 (m, 1H), 9.01 (s, 1H). | | |
| Example 82 | ¹H NMR (400 MHz, DMSO-d₆): δ 2.89 (t, 2H), | ¹⁹F NMR (400 MHz, | LCMS (Method-H): |
| | 3.56-3.75 (m, 8H), 3.83-4.11 (m, 4H), 6.13-6.47 | DMSO-d6): δ −45.646 | ES(+ve): 577.0 |
| | (m, 1H), 6.65 (s, 1H), 7.05 (d, 1H), 7.37-7.43 (m, | (1F), −120.700 (2F). | [M + H]⁺. |
| | 1H), 7.48 (t, 2H), 7.78-7.87 (m, 1H), 7.90-8.03 (m, | | |
| | 3H), 8.53-8.65 (m, 2H), 8.75-8.82 (m, 1H). | | |
| Example 83 | ¹H NMR (400 MHz, DMSO-d₆): δ 2.80-2.93 (br. s, | ¹⁹F NMR (400 MHz, | LCMS (Method-C): |
| | 2H), 3.55-4.12 (br. m, 12H), 6.12-6.47 (m, 1H), | DMSO-d6): δ −58.636 | ES(+ve): 577.4 |
| | 6.64 (s, 1H), 7.05 (s, 1H), 7.23-7.53 (br. m, 4H), | (1F), −120.680 (2F). | [M + H]⁺. |
| | 7.76-8.05 (br. m, 3H), 8.45-8.66 (br. m, 2H), 8.87- | | |
| | 8.98 (br. s, 1H). | | |
| Example 84 | ¹H NMR (400 MHz, DMSO-d₆): δ 2.88 (t, 2H), | ¹⁹F NMR (400 MHz, | LCMS (Method-C): |
| | 3.57-3.74 (m, 8H), 3.83-4.02 (m, 4H), 4.59-4.65 | DMSO-d6): δ −45.634 | ES(+ve): 559.4 |
| | (m, 1H), 4.72-4.77 (m, 1H), 6.60 (s, 1H), 7.04 (d, | (1F), −221.402_−221.517 | [M + H]⁺. |
| | 1H), 7.37-7.43 (m, 1H), 7.47 (t, 2H), 7.80-7.86 (m, | (1F). | |
| | 1H), 7.91-8.04 (m, 3H), 8.52-8.63 (m, 2H), 8.75- | | |
| | 8.82 (m, 1H). | | |
| Example 85 | ¹H NMR (400 MHz, DMSO-d₆): δ 2.87 (t, 2H), | ¹⁹F NMR (400 MHz, | LCMS (Method-C): |
| | 3.56-3.76 (m, 8H), 3.82-4.03 (m, 4H), 4.57-4.64 | DMSO-d6): δ −58.629 | ES(+ve): 559.5 |
| | (m, 1H), 4.70-4.76 (m, 1H), 6.61 (s, 1H), 7.05 (d, | (1F), −221.372_−221.496 | [M + H]⁺. |
| | 1H), 7.27-7.32 (m, 1H), 7.37-7.43 (m, 1H), 7.49 (t, | (1F). | |
| | 2H), 7.78-7.90 (m, 1H), 7.98 (d, 2H), 8.48-8.64 (m, | | |
| | 2H), 8.90-8.97 (m, 1H). | | |
| Example 86 | ¹H NMR (400 MHz, DMSO-d₆): δ 2.91 (t, 2H), | ¹⁹F NMR (400 MHz, | LCMS (Method-C): |
| | 3.58-3.78 (m, 8H), 3.85-4.01 (m, 2H), 4.48-4.66 | DMSO-d6): δ −45.657 | ES(+ve): 595.3 |
| | (m, 2H), 6.67 (s, 1H), 7.06 (d, 1H), 7.37-7.43 (m, | (1F), −68.557 (3F). | [M + H]⁺. |
| | 1H), 7.48 (t, 2H), 7.81-7.86 (m, 1H), 7.91-8.02 (m, | | |
| | 3H), 8.52-8.62 (m, 1H), 8.65 (d, 1H), 8.78 (d, 1H). | | |
| Example 87 | ¹H NMR (400 MHz, DMSO-d₆): δ 2.90 (t, 2H), | ¹⁹F NMR (400 MHz, | LCMS (Method-C): |
| | 3.58-3.78 (m, 8H), 3.83-4.00 (m, 2H), 4.47-4.67 | DMSO-d6): δ −58.635 | ES(+ve): 595.3 |
| | (m, 2H), 6.68 (s, 1H), 7.07 (d, 1H), 7.27-7.32 (m, | (1F), −68.539 (3F). | [M + H]⁺. |
| | 1H), 7.37-7.43 (m, 1H), 7.48 (t, 2H), 7.78-7.87 (m, | | |
| | 1H), 7.98 (d, 2H), 8.48-8.62 (m, 1H), 8.65 (d, 1H), | | |
| | 8.90-8.97 (m, 1H). | | |
| Example 88 | ¹H NMR (400 MHz, DMSO-d₆): δ 2.86 (t, 2H), 3.09 | ¹⁹F NMR (400 MHz, | LCMS (Method-J): |
| | (s, 3H), 3.57-3.63 (m, 4H), 3.67-3.72 (m, 4H), 3.85- | DMSO-d6): δ −45.610 | ES(+ve): 527.3 |
| | 3.94 (m, 2H), 5.66 (s, 1H), 6.97 (d, 1H), 7.33-7.39 | (1F). | [M + H]⁺. |
| | (m, 1H), 7.45 (t, 2H), 7.79-7.83 (m, 1H), 7.94 (d, | | |
| | 2H), 8.04 (s, 1H), 8.58 (s, 1H), 8.62 (d, 1H), 8.75- | | |
| | 8.78 (m, 1H). | | |
| Example 89 | ¹H NMR (400 MHz, DMSO-d₆): δ 2.27 (s, 3H), 2.78 | ¹⁹F NMR (400 MHz, | LCMS (Method-B): |
| | (t, 2H), 3.16 (s, 3H), 3.58-3.75 (m, 8H), 3.80-3.93 | DMSO-d6): δ −58.814 | ES(+ve): 541.1 |
| | (m, 2H), 6.56 (s, 1H), 6.78 (s, 1H), 7.04 (s, 1H), | (1F). | [M + H]⁺. |
| | 7.20-7.26 (m, 1H), 7.37-7.44 (m, 1H), 7.45-7.53 | | |
| | (m, 2H), 7.98 (d, 1H), 8.45 (br. s, 1H), 8.61 (br. s, | | |
| | 1H), 8.85-8.92 (m, 1H). | | |
| Example 90 | ¹H NMR (400 MHz, DMSO-d₆): δ 2.85 (t, 2H), 3.08 | ¹⁹F NMR (400 MHz, | LCMS (Method-B): |
| | (s, 3H), 3.58-3.64 (m, 4H), 3.65-3.72 (m, 4H), 3.86- | DMSO-d6): δ −45.610 | ES(+ve): 527.2 |
| | 3.96 (m, 2H), 5.66 (s, 1H), 7.27 (t, 1H), 7.40 (t, | (1F). | [M + H]⁺. |
| | 2H), 7.74 (d, 2H), 7.78-7.83 (m, 1H), 7.98 (s, 1H), | | |
| | 8.19 (s, 1H), 8.63 (s, 1H), 8.74-8.79 (m, 1H), 8.94 | | |
| | (s, 1H). | | |
| Example 91 | ¹H NMR (400 MHz, DMSO-d₆): δ 2.84 (t, 2H), 3.08 | ¹⁹F NMR (400 MHz, | LCMS (Method-B): |
| | (s, 3H), 3.58-3.64 (m, 4H), 3.65-3.72 (m, 4H), 3.85- | DMSO-d6): δ −58.626 | ES(+ve): 527.2 |
| | 3.95 (m, 2H), 5.66 (s, 1H), 7.23-7.29 (m, 2H), 7.40 | (1F). | [M + H]⁺. |
| | (t, 2H), 7.75 (d, 2H), 7.87 (s, 1H), 8.18 (s, 1H), 8.59 | | |
| | (s, 1H), 8.84-8.92 (m, 1H), 8.94 (s, 1H). | | |
| Example 92 | ¹H NMR (400 MHz, DMSO-d₆): δ 2.87 (d, 2H), | ¹⁹F NMR (400 MHz, | LCMS (Method-B): |
| | 2.92-3.02 (m, 1H), 3.58-3.65 (m, 4H), 3.66-3.72 | DMSO-d6): δ −45.605 | ES(+ve): 539.2 |
| | (m, 4H), 3.75-3.82 (m, 2H), 4.15 (t, 2H), 6.59 (s, | (1F). | [M + H]⁺. |
| | 1H), 7.03 (d, 1H), 7.37-7.42 (m, 1H), 7.44-7.50 (m, | | |
| | 2H), 7.83-7.87 (m, 1H), 7.96-8.00 (m, 3H), 8.53- | | |
| | 8.57 (m, 2H), 8.77-8.82 (m, 1H). | | |
| Example 93 | ¹H NMR (400 MHz, DMSO-d₆): δ 2.87 (d, 2H), | ¹⁹F NMR (400 MHz, | LCMS (Method-B): |
| | 2.92-3.02 (m, 1H), 3.58-3.65 (m, 4H), 3.66-3.72 | DMSO-d6): δ −58.612 | ES(+ve): 539.0 |
| | (m, 4H), 3.75-3.82 (m, 2H), 4.15 (t, 2H), 6.60 (s, | (1F). | [M + H]⁺. |
| | 1H), 7.03 (d, 1H), 7.30-7.33 (m, 1H), 7.37-7.43 (m, | | |
| | 1H), 7.43-7.50 (m, 2H), 7.86 (s, 1H), 7.97 (d, 2H), | | |
| | 8.52-8.57 (m, 2H), 8.92-8.98 (m, 1H). | | |
| Example 94 | ¹H NMR (400 MHz, DMSO-d₆): δ 2.62 (s, 3H), | ¹⁹F NMR (400 MHz, | LCMS (Method-B): |
| | 2.71-2.80 (m, 2H), 3.13 (br. s, 3H), 3.57-3.87 (m, | DMSO-d6): δ −45.147 | ES(+ve): 541.2 |
| | 10H), 6.55 (s, 1H), 7.04 (d, 1H), 7.35-7.42 (m, 1H), | (1F). | [M + H]⁺. |
| | 7.46 (t, 2H), 7.85 (br. s, 2H), 7.96 (d, 2H), 8.60 (br. | | |
| | s, 1H), 8.74 (br. s, 1H). | | |
| Example 95 | ¹H NMR (400 MHz, DMSO-d₆): δ 2.27 (s, 3H), 2.78 | ¹⁹F NMR (400 MHz, | LCMS (Method-B): |
| | (t, 2H), 3.15 (s, 3H), 3.58-3.64 (m, 4H), 3.66-3.72 | DMSO-d6): δ −45.673 | ES(+ve): 541.2 |
| | (m, 4H), 3.82-3.89 (m, 2H), 6.54 (s, 1H), 7.02 (d, | (1F). | [M + H]⁺. |
| | 1H), 7.37-7.42 (m, 1H), 7.43-7.50 (m, 2H), 7.69- | | |
| | 7.75 (m, 1H), 7.96 (d, 2H), 8.46 (br. s, 1H), 8.60 | | |
| | (br. s, 1H), 8.71 (d, 1H). | | |
| Example 96 | ¹H NMR (400 MHz, DMSO-d₆): δ 1.15 (t, 3H), 2.90 | ¹⁹F NMR (400 MHz, | LCMS (Method-B): |
| | (t, 2H), 3.55-3.74 (m, 10H), 3.79-3.90 (m, 2H), | DMSO-d6): δ −44.959 | ES(+ve): 541.0 |
| | 6.57 (s, 1H), 7.04 (d, 1H), 7.37-7.43 (m, 1H), 7.47 | (1F). | [M + H]⁺. |
| | (t, 2H), 7.83-7.93 (m, 2H), 7.97 (d, 2H), 8.57-8.70 | | |
| | (m, 2H), 8.85-8.88 (m, 1H). | | |
| Example 97 | ¹H NMR (400 MHz, DMSO-d₆): δ 1.04-1.12 (m, | ¹⁹F NMR (400 MHz, | LCMS (Method-B): |
| | 3H), 3.37-3.88 (m, 12H), 6.53 (s, 1H), 6.96-7.10 | DMSO-d6): δ −44.751 | ES(+ve): 541.1 |
| | (m, 2H), 7.37-7.42 (m, 1H), 7.43-7.51 (m, 2H), | (1F). | [M + H]⁺. |
| | 7.77-7.87 (m, 1H), 7.98 (d, 2H), 8.30-8.53 (m, 2H), | | |
| | 8.78-8.83 (m, 1H). Two protons obscured by | | |
| | DMSO peak. | | |
| Example 98 | ¹H NMR (400 MHz, DMSO-d₆): δ 1.16 (t, 3H), 2.91 | ¹⁹F NMR (400 MHz, | LCMS (Method-B): |
| | (t, 2H), 3.56-3.73 (m, 10H), 3.80-3.91 (m, 2H), | DMSO-d6): δ −58.487 | ES(+ve): 541.2 |
| | 6.57 (s, 1H), 7.04 (d, 1H), 7.33-7.43 (m, 2H), 7.47 | (1F). | [M + H]⁺. |
| | (t, 2H), 7.72-7.83 (m, 1H), 7.97 (d, 2H), 8.54-8.70 | | |
| | (m, 2H), 8.92-8.99 (m, 1H). | | |
| Example 99 | ¹H NMR (400 MHz, DMSO-d₆): δ 3.04 (t, 2H), | ¹⁹F NMR (400 MHz, | LCMS (Method-B): |
| | 3.72-3.90 (m, 8H), 4.65 (t, 2H), 7.00-7.04 (m, 1H), | DMSO-d6): δ −58.999 | ES(+ve): 514.2 |
| | 7.12 (s, 1H), 7.18 (d, 1H), 7.40-7.46 (m, 1H), 7.50 | (1F). | [M + H]⁺. |
| | (t, 2H), 7.94 (s, 1H), 8.04 (d, 2H), 8.70-8.77 (m, | | |
| | 2H), 8.95 (d, 1H). | | |
| Example 100 | ¹H NMR (400 MHz, DMSO-d₆): δ 3.05 (t, 2H), | ¹⁹F NMR (400 MHz, | LCMS (Method-B): |
| | 3.72-3.87 (m, 8H), 4.67 (t, 2H), 6.96-7.00 (m, 1H), | DMSO-d6): δ −45.007 | ES(+ve): 514.2 |
| | 7.12 (s, 1H), 7.18 (d, 1H), 7.40-7.45 (m, 1H), 7.50 | (1F). | [M + H]⁺. |
| | (t, 2H), 7.95 (s, 1H), 8.04 (d, 2H), 8.45-8.48 (m, | | |
| | 1H), 8.71 (d, 1H), 8.95 (d, 1H). | | |
| Example 101 | ¹H NMR (400 MHz, DMSO-d₆): δ 2.85 (t, 2H), 3.16 | — | LCMS (Method-B): |
| | (s, 3H), 3.57-3.75 (m, 8H), 3.83-3.98 (m, 2H), 5.54 | | ES(+ve): 534.2 |
| | (d, 1H), 6.33 (d, 1H), 6.57 (s, 1H), 6.80-6.90 (m, | | [M + H]⁺. |
| | 1H), 7.06 (d, 1H), 7.37-7.43 (m, 1H), 7.48 (t, 2H), | | |
| | 7.66-7.71 (m, 1H), 7.80 (br. s, 1H), 7.89 (s, 1H), 7.99 | | |
| | (d, 2H), 8.56 (d, 1H), 8.61 (br. s, 1H), 8.66 (d, 1H). | | |
| Example 102 | ¹H NMR (400 MHz, DMSO-d₆): δ 2.03-2.13 (m, | ¹⁹F NMR (400 MHz, | LCMS (Method-B): |
| | 1H), 2.27-2.37 (m, 1H), 3.01-3.11 (m, 1H), 3.56- | DMSO-d6): δ −45.600 | ES(+ve): 538.8 |
| | 3.75 (m, 8H), 3.78-3.97 (m, 2H), 4.52-4.61 (m, | (1F). | [M + H]⁺. |
| | 1H), 6.66 (s, 1H), 7.03 (d, 1H), 7.37-7.43 (m, 1H), | | |
| | 7.48 (t, 2H), 7.85-7.89 (m, 1H), 7.92-8.02 (m, 3H), | | |
| | 8.53-8.60 (m, 2H), 8.79-8.83 (m, 1H). One proton | | |
| | obscured by water peak. | | |
| Example 103 | ¹H NMR (400 MHz, DMSO-d₆): δ 2.03-2.13 (m, | ¹⁹F NMR (400 MHz, | LCMS (Method-B): |
| | 1H), 2.27-2.36 (m, 1H), 3.01-3.10 (m, 1H), 3.56- | DMSO-d6): δ −58.593 | ES(+ve): 539.2 |
| | 3.75 (m, 8H), 3.77-3.97 (m, 2H), 4.52-4.61 (m, | (1F). | [M + H]⁺. |
| | 1H), 6.65 (s, 1H), 7.03 (d, 1H), 7.30-7.34 (m, 1H), | | |
| | 7.37-7.43 (m, 1H), 7.47 (t, 2H), 7.82 (s, 1H), 7.98 | | |
| | (d, 2H), 8.52-8.57 (m, 2H), 8.92-8.97 (m, 1H). One | | |
| | proton obscured by water peak. | | |
| Example 104 | ¹H NMR (400 MHz, DMSO-d₆): δ 2.03-2.13 (m, | ¹⁹F NMR (400 MHz, | LCMS (Method-B): |
| | 1H), 2.27-2.37 (m, 1H), 3.01-3.10 (m, 1H), 3.56- | DMSO-d6): δ −45.604 | ES(+ve): 538.8 |
| | 3.75 (m, 8H), 3.78-3.96 (m, 2H), 4.52-4.61 (m, | (1F). | [M + H]⁺. |
| | 1H), 6.65 (s, 1H), 7.02 (d, 1H), 7.37-7.43 (m, 1H), | | |
| | 7.47 (t, 2H), 7.84-7.88 (m, 1H), 7.92-8.02 (m, 3H), | | |
| | 8.53-8.60 (m, 2H), 8.78-8.82 (m, 1H). One proton | | |
| | obscured by water peak. | | |
| Example 105 | ¹H NMR (400 MHz, DMSO-d₆): δ 2.03-2.13 (m, | ¹⁹F NMR (400 MHz, | LCMS (Method-B): |
| | 1H), 2.27-2.36 (m, 1H), 3.01-3.10 (m, 1H), 3.56- | DMSO-d6): δ −58.583 | ES(+ve): 539.2 |
| | 3.75 (m, 8H), 3.77-3.97 (m, 2H), 4.52-4.61 (m, | (1F). | [M + H]⁺. |
| | 1H), 6.66 (s, 1H), 7.03 (d, 1H), 7.30-7.34 (m, 1H), | | |
| | 7.37-7.44 (m, 1H), 7.48 (t, 2H), 7.83 (s, 1H), 7.99 | | |
| | (d, 2H), 8.52-8.59 (m, 2H), 8.92-8.99 (m, 1H). One | | |
| | proton obscured by water peak. | | |

### Biological Activity

### Example 49: PIKfyve Protein Expression and Purification

Full length wild type PIKfyve (aa 1-2098; UniProt ID: Q9Y217-1) construct with N-terminal 3×FLAG and C-terminal His tag was cloned into pcDNA3.1 vector and transfected into Expi293F cells (Thermo, Cat. No. A14527). Protein was expressed for 72 hours at 30° C. after transfection. The cells were harvested by centrifugation and flash-frozen for storage. Frozen cell pellets were re-suspended in ice-cold lysis buffer (50 mM HEPES, 500 mM NaCl, 10% Glycerol, 0.1% Tween 20, 1 mM TCEP, 1/200 (v/v) Halt™ protease inhibitor cocktail (Thermo, Cat. No. 78429), 0.1 mg Benzonase/1L culture, pH 8.0) and transferred into a Dounce homogenizer and lysed with 10 strokes. The supernatant after centrifugation was incubated with Anti-FLAG-G1 affinity resin (GenScript, Cat. No. L00432) for 1.5 hours. Then, the resin was washed 3 times with 10× bed volumes of wash buffer (50 mM HEPES, 300 mM NaCl, 10% Glycerol, 1 mM TCEP, pH 8.0) and eluted 3 times with 2x bed volume of elution buffer (50 mM HEPES, 300 mM NaCl, 10% Glycerol, 1 mM TCEP, 300 μg/mL FLAG peptide [GenScript, Cat. No. RP10586], pH 8.0). The elution fractions were pooled and further purified using a HiLoad 16/600 Superose™ 6 μg (Cytiva, Cat. No. 29323952) gel filtration column pre-equilibrated with gel filtration buffer (50 mM HEPES, 600 mM NaCl, 5% Glycerol, 0.5 mM TCEP, pH 8.0). Peak fractions of interest were collected and flash frozen in liquid nitrogen for storage at −80° C. Quality control after one freeze-thaw cycle was performed to confirm protein was a monodisperse sample with no indication of aggregation or oligomerization using a Superose™ 6 Increase 5/150 GL (Cytiva, Cat. No. 29091597) gel filtration column pre-equilibrated with gel filtration buffer.

### Example 50: Enzymatic Assay for Inhibition of PIKfyve

The inhibition of Recombinant PIKfyve protein [hPIKfyve (1-2098) or commercial hPIKfyve (Carna, cat no. 11-118)] was measured using ADP-Glo kinase-assay kit (Promega). 300 nl of compounds (dissolved in DMSO) in serial dilution were transferred to 384-well Optiplates (Perkin Elmer) using Echo 650 series liquid handler (Beckman Coulter). 5 μl of Recombinant PIkfyve in assay buffer (25 mM HEPES pH 7.5, 10 mM MgCl₂, 1 μM CaCl₂, 2 mM DTT, 0.05% BSA, and 0.002% Triton-X₁₀₀) is added to the compounds and preincubated for 120 minutes at room temperature. To initiate the reaction, 5 μl of ATP and PI (3) P: PS substrate are added. For hPIKfyve (1-2098) the final concentration of recombinant protein, ATP, and Substrate are 5 nM, 20 μM, and 62.5:500 μM, respectively while for hPIKfyve (Carna), the final concentration of recombinant protein, ATP, and Substrate are 5 nM, 50 μM, and 62.5:500 μM, respectively. The plate was incubated for 90 minutes at room temperature then 10 μl of the ADP-GLO™ reagent was added and incubated for 60 minutes. 20 μl of the kinase detection reagent was added and incubated for an additional 30 minutes then luminescence (LUM) was detected using the Pherastar microplate reader (BMG Labtech). The enzyme activity was normalized using control wells containing negative control (0% inhibition) of 3% DMSO or positive control (100% inhibition) of 10 μM YM-201636 (MedChemExpress).

% Inhibition=100×[1−(LUM_{cmpd}−LUMₚₒₛ)/(LUM_{neg}−LUMₚₒₛ)]

Where LUM_{cmpd}, LUMₚₒₛ, and LUM_{neg} are the relative luminescence units of compound, YM-201636, and DMSO treated wells respectively. The % inhibition values were plotted as a function of compound concentration and the IC₅₀ values of compounds were determined using a four-parameter logistic fit Y=Min+[(Max−Min)/(1+(X/IC₅₀)^{Hill slope})] or three-parameter fit if Max or Min of curves were fixed to 100% or 0% respectively.

Table 1 shows the activity of exemplary compounds against two forms of the PIKfyve enzyme using the ADP-Glo assay format described above, where ‘A’, ‘B’ and ‘C’ have the following meaning:

$$‘ A ’ : {IC}_{50} \leq 0.01 µM$$

$$‘ B ’ : {IC}_{50} > 0.01 µM and \leq 0.1 µM$$

$$‘ C ’ : {IC}_{50} > 0.1 µM and \leq 0.5 µM$$

TABLE 1

| | ADP-Glo Assay vs | |
|---|---|---|
| | hPIKfyve (Carna, cat | ADP-Glo Assay vs |
| | no. 11-118) | hPIKfyve (1-2098) |
| | PIKfyve IC50 (μM) | PIKfyve IC50 (μM) |
| Example 1 | A | — |
| Example 2 | A | — |
| Example 3 | A | — |
| Example 4 | A | — |
| Example 5 | A | — |
| Example 6 | A | — |
| Example 7 | A | — |
| Example 8 | A | A |
| Example 9 | A | — |
| Example 10 | A | A |
| Example 11 | A | — |
| Example 12 | A | — |
| Example 13 | B | — |
| Example 14 | A | — |
| Example 15 | A | A |
| Example 16 | A | A |
| Example 17 | A | A |
| Example 18 | — | A |
| Example 19 | — | C |
| Example 20 | — | B |
| Example 21 | — | A |
| Example 22 | — | B |
| Example 23 | — | B |
| Example 24 | — | B |
| Example 25 | — | B |
| Example 26 | — | C |
| Example 27 | — | A |
| Example 28 | — | C |
| Example 29 | — | B |
| Example 30 | — | C |
| Example 31 | — | A |
| Example 32 | — | A |
| Example 33 | — | B |
| Example 34 | — | B |
| Example 35 | — | A |
| Example 36 | — | A |
| Example 37 | — | A |
| Example 38 | — | B |
| Example 39 | — | A |
| Example 40 | — | A |
| Example 41 | — | C |
| Example 42 | — | B |
| Example 43 | — | B |
| Example 44 | — | B |
| Example 45 | — | C |
| Example 46 | — | B |
| Example 47 | — | B |
| Example 48 | — | C |

While this invention has been particularly shown and described with references to preferred embodiments thereof, it will be understood by those skilled in the art that various changes in form and details may be made therein without departing from the scope of the invention encompassed by the appended claims.

What is claimed:

1. A compound of formula (I): 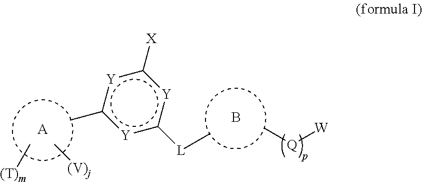
or a pharmaceutically acceptable salt thereof, wherein Ring A is a 5- or 6-membered aromatic ring having 0-3 heteroatoms selected from N, O, or S;
T is selected from halogen, C₁-C₆ alkyl, alkoxy or an aryl or heteroaryl having 0-3 heteroatoms selected from N, O, or S;
V is a C₁-C₃ alkyl, wherein the C₁-C₃ alkyl is optionally substituted; each Y is independently CR₁, N, O, or S, provided at least one Y is N, O, or S, wherein R₁ is H, D, halogen, or substituted or unsubstituted C₁-C₆ alkyl;
X is a 5- to 8-membered heterocyclic ring comprising at least one heteroatom selected from N or O, wherein the ring is optionally substituted, wherein the ring is optionally fused or bridged;
L is —X₁—(CR₃R₄)ₖ—, —(CR₃R₄)ₖ—, —X₁—(CR₃R₄)ₖ—X₁—, —(CR₃R₄)ₖ—X₁—, —(CR₃R₄)ₖ—X₁—(CR₃R₄) k-; each X₁ is independently selected from —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂—, and —NR₂C(Z₁) NR₂—; wherein each occurrence of Z₁ is independently O or NR₆, preferably Z₁ is O; preferably-O—, —C(O)—, —C(O)O—, —OCO—, —S—, —S(O)₂—, —S(O)—, —S(O)(NR₂)—, —S(O)₂ (NR₂)—, —NR₂—, —NR₂C(O)—, and —C(O)NR₂—;
R₂ is hydrogen, substituted or unsubstituted C₁₋₆ alkyl, substituted or unsubstituted C₃₋₇ cycloalkyl, or substituted or unsubstituted C₃₋₇ heterocycloalkyl;
R₆ is hydrogen, substituted or unsubstituted C₁₋₆ alkyl, substituted or unsubstituted C₃₋₇ cycloalkyl, substituted or unsubstituted C₃₋₇ heterocycloalkyl or acyl;
each occurrence of R₃ and R₄ are independently H, halogen, hydroxy, substituted or unsubstituted alkyl, substituted or unsubstituted alkenyl, substituted or unsubstituted alkynyl, or substituted or unsubstituted alkoxy; alternatively, at least one occurrence of R₃ and R₄ forms an optionally substituted cycloalkyl or heterocycloalkyl; alternatively, when X₁ comprises-NR₂—, any two of one R₃, one R₄, and R₂ forms an optionally substituted heterocycloalkyl;
Ring B is a substituted or unsubstituted 5- or 6-membered aromatic ring having 0-3 heteroatoms selected from N, O, or S;
Q is selected from —CR₃R₄—, —O—, —C(Z₁)—, —C(Z₁)O—, —OC(Z₁)—, —S—, —S(Z₁)ₓ—, —S(Z₁)ₓNR₂—, —NR₂S(Z₁)ₓ—, —NR₂—, —NR₂C(Z₁)—, —C(Z₁) NR₂—; —NR₂C(Z₁)O—, —OC(Z₁) NR₂— and —NR₂C(Z₁) NR₂; wherein each occurrence of Z₁ is independently O or NR₆;
W is substituted or unsubstituted aryl or substituted or unsubstituted heteroaryl, wherein the heteroaryl comprises one or more heteroatoms selected from N, O, or S, wherein at least one substituent is a halogen, —SR₂, —S(Z₁)ₓR₂—S(Z₁)ₓNR₂R₆, —NR₂S(Z₁) ₓR₆, —OS(Z₁)R₆, substituted or unsubstituted alkene or substituted or unsubstituted alkyne; or when W is attached to N, W is substituted or unsubstituted alkenoyl or substituted or unsubstituted alkynoyl, substituted or unsubstituted alkenesulfonyl; substituted or unsubstituted alkenesulfinyl; substituted or unsubstituted alkynesulfonyl, substituted or unsubstituted alkynesulfinyl, W is preferably a substituted or unsubstituted aryl, a substituted or unsubstituted heteroaryl, wherein the heteroaryl comprises one or more heteroatoms selected from N, O, or S;
m is 0 or 1;
j is 0 or 1;
p is 0 or 1;
k is 0, 1, 2, 3, 4, 5, or 6 and
x is 1 or 2.

2. The compound according to claim 1, wherein ring A is selected from: 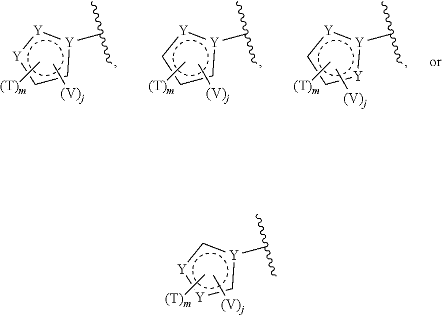
wherein the squiggly lines indicate the point of attachment to the rest of the molecule.

3. The compound according to claim 1, wherein Ring A is:

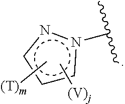

4. The compound of claim 3, wherein m is 1 and Tis substituted or unsubstituted phenyl or substituted or unsubstituted pyridine.

5. The compound according to claim 4, wherein T is selected from phenyl, 3-methylphenyl, 3-fluorophenyl, 3-chlorophenyl, pyridine, 3-methylpyridine, 3-fluoro pyridine, and 3-chloro pyridine.

6. The compound according to claim 1, wherein the moiety 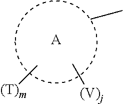
is selected from:

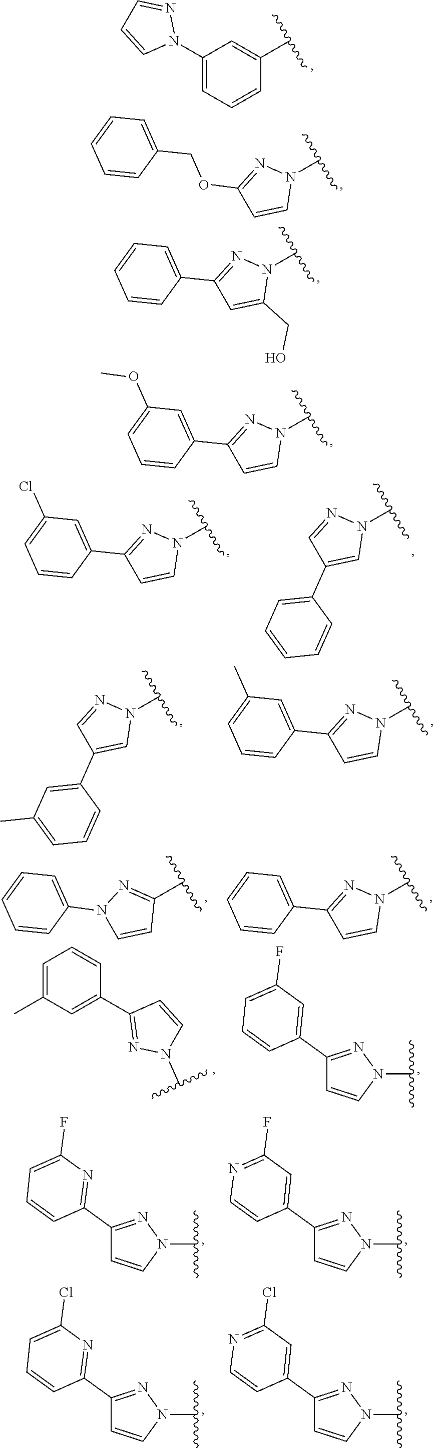

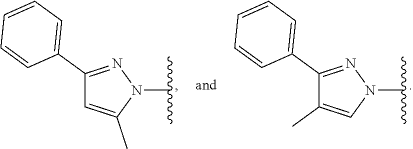 [SMILES: *n1nc(-c2ccccc2)cc1C ; *n1cc(C)c(-c2ccccc2)n1]

7. The compound according to claim 1, wherein the moiety 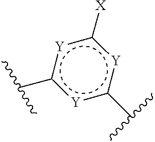
is selected from:

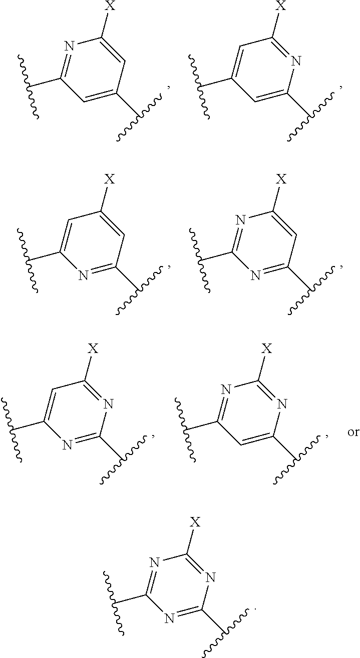

8. The compound according to claim 7, wherein X is selected from: 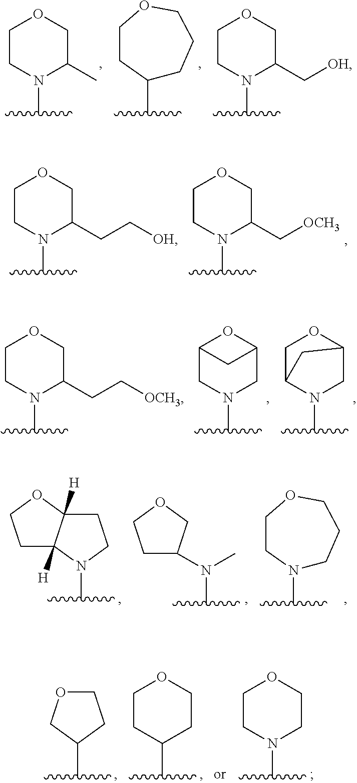
wherein each of the rings above is optionally substituted, and the substituents are independently selected from halogen, —OH, a C₁₋₄ alkyl, and a C₁₋₄ alkoxy.

9. The compound according to claim 7, wherein X is:

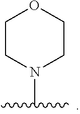 [SMILES: *N1CCOCC1]

10. The compound according to claim 1, wherein L is —X₁—(CR₃R₄)ₖ—.

11. The compound according to claim 1, wherein L is —O—(CR₃R₄)₁₋₄—, wherein each R₃ and R₄ are independently selected from H, D, halogen, C₁₋₄ alkyl; or L is —NR₂—(CR₃R₄)₁₋₄—, wherein each R₃ and R₄ are independently selected from H, D, halogen, C₁₋₄ alkyl.

12. The compound according to claim 1, wherein L is —O—(CH₂)₁₋₄—.

13. The compound according claim 1, wherein the compound is of formula IIa or IIb: 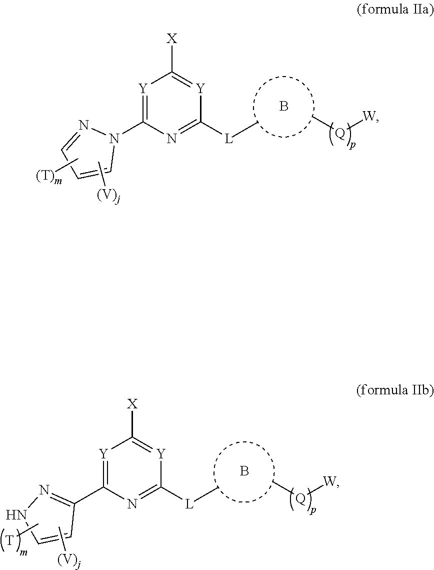
or a pharmaceutically acceptable salt thereof.

14. The compound according to claim 1, wherein Ring B is selected from:

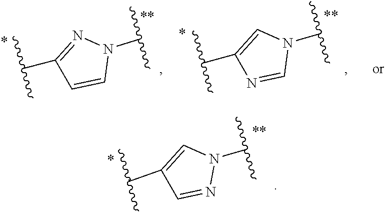 [SMILES: *c1ccn(*)n1 ; *c1cn(*)cn1 ; *c1cnn(*)c1]

15. The compound according to claim 1, wherein p is 0.

16. The compound according to claim 1, wherein p is 1, and Q is —S(O)₂—.

17. The compound according to claim 1, wherein the compound is of formula Va: 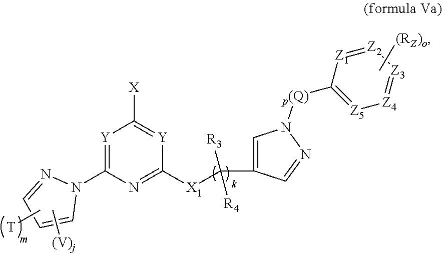
or a pharmaceutically acceptable salt thereof.

18. The compound according to claim 17, wherein the compound is of formula VIa: 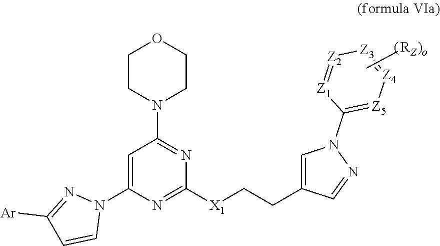
or a pharmaceutically acceptable salt thereof.

19. A compound selected from a compound from Table 1.
